(19) United States
(12) Patent Application Publication
(10) Pub. No.: US 2002/0103192 A1
(43) Pub. Date: Aug. 1, 2002

(54) Inhibitors of histone deacetylase

(76) Inventors: Michael L. Curtin, Pleasant Prairie, WI (US); Steven K. Davidsen, Libertyville, IL (US); Robin R. Frey, Libertyville, IL (US); Howard R. Heyman, Deerfield, IL (US); Michael R. Michaelides, Libertyville, IL (US); Anil Vasudevan, Gurnee, IL (US); Carol K. Wada, Gurnee, IL (US)

Correspondence Address: Steven F. Weinstock, Abbott Laboratories, AP6D/D-377, 100 Abbott Park Road, Abbott Park, IL, 60064-6050, US

(21) Appl. No.: 09/808,389

(22) Filed: Mar. 14, 2001

Related U.S. Application Data
(63) Continuation-in-part of application No. 09/697,387, filed on Oct. 26, 2000.

(51) Int. Cl. C11C 3/00; A61K 31/54; A61K 31/541; A61K 31/5375; A61K 31/5377; A61K 31/495; A61K 31/496; A61K 31/421; A61K 31/426; A61K 31/277; A61K 31/16

(52) U.S. Cl. 514/227.8; 514/227.5; 514/236.8; 514/254.02; 514/254.03; 514/364; 514/365; 514/374; 514/521; 514/625; 514/466; 544/60; 549/441; 558/418; 554/36

(57) ABSTRACT

Substituted trifluoromethyl ketones, ketooxazoles, ketoamides, ketoesters, and diketones having the formula

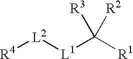

or therapeutically acceptable salts thereof, are histone deacetylase (HDAC) inhibitors. Preparation of the compounds, compositions containing the compounds, and treatment of diseases using the compounds are disclosed.

### CROSS-REFERENCE TO RELATED APPLICATIONS

This application is a continuation-in-part of U.S. patent application Ser. No. 09/697,387, filed Oct. 26, 2000, which is herein incorporated by reference.

### TECHNICAL FIELD

The present invention relates to substituted trifluoromethyl ketones, ketooxazoles, ketoamides, ketoesters, and diketones which are useful for inhibiting histone deacetylase (HDAC), methods of making the compounds, compositions containing the compounds, and methods of treatment using the compounds.

### BACKGROUND OF THE INVENTION

The organized packing of DNA in the eukaryotic nucleus plays an important role in the regulation of gene transcription. DNA's highly condensed state is a consequence of its wrapping into chromatin. The fundamental repeating structural unit of chromatin is the nucleosome, which consists of 146 bases of DNA wrapped around a complex of eight histone proteins, two molecules each of the core histones, H2A, H2B, H3, and H4. Each core histone octomer is comprised of several highly conserved structural motifs including a globular domain and an N-terminal tail domain that extends outside of the nucleosome. These histone N-terminal tails are enriched in basic amino acids, and are thought to mediate histone-DNA contacts through electrostatic interactions with DNA's negatively charged phosphate backbone. Based on the x-ray crystal structure of the nucleosome core particle, N-terminal histone tails also form contacts with the surface of histones of neighboring nucleosomes.

The capacity of histones to compact DNA is influenced by a number of post-translational modifications that occur on the N-terminal histone tails. One modification involves the reversible acetylation and deacetylation of the epsilon-amino group of lysine moieties found within the histone tails. The net level of acetylation of N-terminal histone tails is controlled by the activities of two families of enzymes, the histone acetyltransferases (HATs) and histone deacetylases (HDACs). The identification of coactivator complexes that possess intrinsic HAT activity strongly supports the connection between histone acetylation and transcriptional activation (Bioessays 1998, 20, 615). Similarly, transcriptional repressor complexes have been shown to recruit HDACs to the promoter of target genes.

Several human cancers have been associated with malfunctions in HAT and HDAC activity. One example is the translocation of chromosomes 15 and 17 seen in the majority of acute promyelocytic leukemia patients. This translocation leads to the formation of a chimeric protein composed of the retinoic acid receptor fused to the PML transcription factor (PML-RARa) (Mol. and Cell. Bio. 1998, 18, 7176). The recruitment of HDACs by this fusion protein diminishes its responsiveness to retinoic acid resulting in inhibition of differentiation of hematopoietic cells, one of the characteristic features of this disease.

Inhibition of the action of HDACs causes a variety of cellular responses including the accumulation of hyperacetylated histones, altered gene expression, and cell cycle arrest. Antiproliferative and antitumor properties have also been described for compounds possessing HDAC inhibitory activity (J. Biol. Chem. 1999, 274, 34940). While a number of natural product and synthetic HDAC inhibitors have been reported (J. Med.Chem. 1999, 42, 3001; and PNAS, 1998, 95, 3003), there still exists a need for inhibitors with improved profiles of activity.

### SUMMARY OF THE INVENTION

In its principle embodiment, therefore, the present invention provides a compound of formula (I)

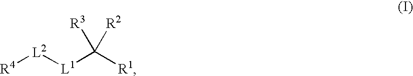

or a therapeutically acceptable salt thereof, wherein

L¹ is selected from the group consisting of alkenylene, alkylene, alkynylene, cycloalkylene, heteroalkylene, and -(alkylene)C(O)N(R⁵)(alkylene)-;

wherein each group is drawn with its left-hand end being the end which attaches to L², and

its right-hand end being the end which attaches to the carbon substituted with R¹, R², and R³.

L is selected from the group consisting of C₂ alkenylene, —O—, —S—, —SO₂—, —OC(O)NR⁵—, —N(R⁶)C(O)—, and —C(O)N(R⁶)—,

wherein each group is drawn with its left-hand end being the end which attaches to R⁴, and its right-hand end being the end which attaches to L¹;

with the proviso that the distance between R⁴ and the carbon substituted with R¹, R², and R³ does not exceed 10 atoms;

R¹ is selected from the group consisting of alkanoyl, alkoxycarbonyl, aminocarbonyl, carboxy, perfluoroalkyl, and heterocycle, wherein the heterocycle is selected from the group consisting of oxazolyl, dihydrooxazolyl, and oxadiazole;

R² and R³ are hydroxy; or

R² and R³ together are oxo;

R⁴ is selected from the group consisting of alkoxyalkyl, alkyl, aryl, arylalkyl, cycloalkyl, (cycloalkyl)alkyl, heterocycle, and (heterocycle)alkyl; and

R⁵ and R⁶ are independently selected from the group consisting of hydrogen, alkyl, aryl, and arylalkyl; or

R⁴ and R⁶, together with the nitrogen atom to which they are attached, form a heterocycle selected from the group consisting of morpholinyl, piperazinyl, piperidinyl, and thiomorpholinyl; wherein the morpholinyl, the piperazinyl, the piperidinyl, and the thiomorpholinyl can be optionally substituted with one, two, or three substituents independently selected from the group consisting of alkyl and spiroheterocycle.

In another embodiment, the present invention provides a pharmaceutical composition comprising a compound of formula (I), or a therapeutically acceptable salt thereof, in combination with a therapeutically acceptable carrier.

In another embodiment, the present invention provides a method of inhibiting histone deacetylase in a patient in recognized need of such treatment comprising administering to the patient a therapeutically acceptable amount of a compound of formula (I), or a therapeutically acceptable salt thereof.

### DETAILED DESCRIPTION OF THE INVENTION

Compounds of the present invention comprise substituted trifluoromethyl ketones, ketooxazoles, ketoamides, ketoesters, and diketones which are useful for the treatment of diseases in which histone deacetylase plays a role.

As used in the present specification the following terms have the meanings indicated:

The term “alkanoyl,” as used herein, represents an alkyl group attached to the parent molecular moiety through a carbonyl group.

The term “alkenylene,” as used herein, represents a divalent group of two to ten carbon atoms derived from a straight or branched chain hydrocarbon containing at least one double bond.

The term “C₂ alkenylene,” as used herein, represents a divalent group of two carbon atoms containing a double bond.

The term “C₆ alkenylene”, as used herein, represents a divalent group of six carbon atoms containing at least one double bond.

The term “alkoxy,” as used herein, represents an alkyl group attached to the parent molecular moiety through an oxygen atom.

The term “alkoxyalkyl,” as used herein, represents an alkoxy group attached to the parent molecular moiety through an alkyl group.

The term “alkoxycarbonyl,” as used herein, represents an alkoxy group attached to the parent molecular moiety through a carbonyl group.

The term “alkyl,” as used herein, represents a monovalent group of one to ten carbon atoms derived from a straight or branched chain saturated hydrocarbon by the removal of a single hydrogen atom.

The term “alkylene,” as used herein, represents a divalent group of one to ten carbon atoms derived from a straight or branched chain saturated hydrocarbon. The alkylene groups of the present invention can be optionally substituted with a hydroxy group.

The term “C₅-C₇ alkylene,” as used herein, represents a divalent group of five to seven carbon atoms derived from a straight or branched chain saturated hydrocarbon. The C₅-C₇ alkylene groups of the present invention can be optionally substituted with a hydroxy group.

The term “C₆ alkylene,” as used herein, represents a divalent group of six carbon atoms derived from a straight or branched chain saturated hydrocarbon. The C₆ alkylene groups of the present invention can be optionally substituted with a hydroxy group.

The term “alkylsulfonyl,” as used herein, represents an alkyl group attached to the parent molecular moiety through a sulfonyl group.

The term “alkynylene,” as used herein, represents a divalent group of two to ten carbon atoms derived from a straight or branched chain hydrocarbon containing at least one triple bond.

The term “C₆ alkynylene,” as used herein, represents a divalent group of six carbon atoms derived from a straight or branched chain hydrocarbon containing at least one triple bond.

The term “amino,” as used herein, represents —NR⁷R⁸, wherein R⁷ and R8 are independently selected from the group consisting of hydrogen, alkanoyl, alkyl, cycloalkyl, (cycloalkyl)alkyl, a nitrogen protecting group, and unsubstituted phenyl; or R⁷ and R⁸, together with the nitrogen atom to which they are attached, form a heterocycle selected from the group consisting of morpholinyl, piperazinyl, piperidinyl, pyrrolidinyl, thiomorpholinyl, and thiomorpholinyl dioxide.

The term “aminocarbonyl,” as used herein, represents an amino group attached to the parent molecular moiety through a carbonyl group.

The term “aryl,” as used herein, represents dihydronaphthyl, fluorenyl, indanyl, indenyl, naphthyl, phenyl, and tetrahydronaphthyl. The aryl groups of the present invention can be optionally substituted with one, two, three, four, or five substituents independently selected from the group consisting of alkanoyl, alkoxy, alkyl, alkylsulfonyl, amino, carbonyloxy, cyano, cycloalkyl, (cycloalkyl)alkyl, halo, haloalkyl, heterocycle, (heterocycle)alkyl, hydroxy, nitro, oxo, perfluoroalkoxy, perfluoroalkyl, phenoxy, phenyl, phenylalkoxy, phenylalkyl, phenyloyl, phenylsulfonyl, thioalkoxy, and thiophenoxy, wherein the phenyl and the phenyl part of the phenoxy, the phenylalkoxy, the phenylalkyl, and the phenyloyl can be further optionally substituted with one, two, or three substituents independently selected from the group consisting of alkoxy, alkyl, alkylsulfonyl, amino, cyano, halo, hydroxy, nitro, perfluoroalkoxy, perfluoroalkyl, phenylsulfonyl, and thioalkoxy.

The term “arylalkoxy,” as used herein, represents an aryl group attached to the parent molecular moiety through an alkoxy group.

The term “arylalkyl,” as used herein, represents an aryl group attached to the parent molecular moiety through an alkyl group. The alkyl part of the arylalkyl groups of the present invention can be optionally substituted with one or two substituents independently selected from the group consisting of aminocarbonyl and aryl.

The term “carbonyl,” as used herein, represents —C(O)—.

The term “carbonyloxy,” as used herein, represents an alkanoyl group attached to the parent molecular moiety through an oxygen atom.

The term “carboxy,” as used herein, represents —CO₂H.

The term “cyano,” as used herein, represents —CN.

The term “cycloalkyl,” as used herein, represents a saturated monocyclic, bicyclic, or tricyclic hydrocarbon ring system having three to twelve carbon atoms. Examples of cycloalkyl groups include cyclopropyl, cyclopentyl, bicyclo(3.1.1)heptyl, adamantyl, and the like.

The term “(cycloalkyl)alkyl,” as used herein, represents a cycloalkyl group attached to the parent molecular moiety through an alkyl group.

The term “cycloalkylene,” as used herein represents a divalent group derived from a saturated monocyclic hydrocarbon ring system having three to twelve carbon atoms.

The term “halo,” as used herein, represents F, Cl, Br, or I.

The term “haloalkyl,” as used herein, represents an alkyl group substituted by one, two, three, or four halogen atoms.

The term “heteroalkylene,” as used herein, represents a divalent group of two to eight atoms derived from a saturated straight or branched chain containing one or two heteroatoms independently selected from the group consisting of nitrogen, oxygen, and sulfur, wherein the remaining atoms are carbon. The heteroalkylene groups of the present invention can be attached through the carbon atoms or the heteroatoms in the chain.

The term “heterocycle,” as used herein, represents a five-, six-, or seven-membered ring containing one, two, or three heteroatoms independently selected from the group consisting of nitrogen, oxygen, and sulfur. The five-membered ring has zero to two double bonds and the six- and seven-membered rings have zero to three double bonds. The term “heterocycle” also includes bicyclic groups in which the heterocycle ring is fused to an aryl group. The heterocycle groups of the present invention can be attached through a carbon atom or a nitrogen atom in the group. Examples of heterocycles include, but are not limited to, furyl, thienyl, pyrrolyl, pyrrolidinyl, oxazolyl, dihydrooxazolyl, thiazolyl, irnidazolyl, imidazolinyl, pyrazolyl, isoxazolyl, isothiazolyl, piperidinyl, morpholinyl, thiomorpholinyl, piperazinyl, pyridinyl, indolyl, indolinyl, benzothienyl, and the like. The heterocycle groups of the present invention can be optionally substituted with one, two, three, or four substituents independently selected from the group consisting of alkanoyl, alkoxy, alkyl, amino, carbonyloxy, cyano, cycloalkyl, (cycloalkyl)alkyl, halo, haloalkyl, hydroxy, nitro, oxo, perfluoroalkoxy, perfluoroalkyl, phenoxy, phenyl, phenylalkoxy, phenylalkyl, phenyloyl, thioalkoxy, and thiophenoxy, wherein the phenyl and the phenyl part of the phenoxy, the phenylalkoxy, the phenylalkyl, and the phenyloyl can be further optionally substituted with one, two, or three substituents independently selected from the group consisting of alkoxy, alkyl, amino, cyano, halo, hydroxy, nitro, perfluoroalkoxy, perfluoroalkyl, and thioalkoxy.

The term “(heterocycle) alkyl,” as used herein, represents a heterocycle group attached to the parent molecular group through an alkyl group.

The term “hydroxy,” as used herein, represents —OH.

The term “nitro,” as used herein, represents —NO₂.

The term “nitrogen protecting group,” as used herein, represents groups intended to protect an amino group against undesirable reactions during synthetic procedures. Common N-protecting groups comprise acyl groups such as acetyl, benzoyl, 2-bromoacetyl, 4-bromobenzoyl, tert-butylacetyl, carboxaldehyde, 2-chloroacetyl, 4-chlorobenzoyl, α-chlorobutyryl, 4-nitrobenzoyl, o-nitrophenoxyacetyl, phthalyl, pivaloyl, propionyl, trichloroacetyl, and trifluoroacetyl; sulfonyl groups such as benzenesulfonyl, and p-toluenesulfonyl; carbamate forming groups such as benzyloxycarbonyl, benzyloxycarbonyl (Cbz), tert-butyloxycarbonyl (Boc), p-chlorobenzyloxycarbonyl, p-methoxybenzyloxycarbonyl, and the like.

The term “oxo,” as used herein, represents (=O).

The term “perfluoralkoxy,” as used herein, represents a perfluoroalkyl group attached to the parent molecular moiety through an oxygen atom.

The term “perfluoroalkyl,” as used herein, represents an alkyl group wherein each hydrogen radical bound to the alkyl group has been replaced by a fluoride radical.

The term “phenoxy,” as used herein, represents a phenyl group attached to the parent molecular moiety through an oxygen atom.

The term “phenylalkoxy,” as used herein, represents a phenyl group attached to the parent molecular moiety through an alkoxy group.

The term “phenylalkyl,” as used herein, represents a phenyl group attached to the parent molecular moiety through an alkyl group.

The term “phenyloyl,” as used herein, represents a phenyl group attached to the parent molecular moiety through a carbonyl group.

The term “phenylsulfonyl,” as used herein, represents a phenyl group attached to the parent molecular moiety through a sulfonyl group.

The term “spiroheterocycle,” as used herein, represents a heteroalkylene diradical, each end of which is attached to the same carbon atom of the parent molecular moiety. Examples of spiroheterocycles include dioxolanyl, tetrahydrofuranyl, pyrrolidinyl, and the like.

The term “sulfonyl,” as used herein, represents —SO₂—.

The term “thioalkoxy,” as used herein, represents an alkyl group attached to the parent molecular moiety through a sulfur atom.

The term “thiophenoxy,” as used herein, represents a phenyl group attached to the parent molecular moiety through a sulfur atom.

The present compounds can also exist as therapeutically acceptable prodrugs. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to parent compounds of formula (I) for example, by hydrolysis in blood.

The compounds of the present invention can exist as therapeutically acceptable salts. The term “therapeutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate, lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethyl amine, 1-ephenamine, and N,N ′-dibenzylethylenediamine. Other representative organic amines useful for the formation of base addition salts include ethylenediamine, ethanolamine, diethanolamine, piperidine, and piperazine.

In addition to the compounds of the present invention and their pharmaceutically acceptable salts, the invention is further directed, where applicable, to unsolvated as well as solvated forms of the compounds (e.g. hydrated forms) having the ability to inhibit HDAC.

In accordance with methods of treatment and pharmaceutical compositions of the invention, the compounds can be administered alone or in combination with other HDAC inhibitors. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The inhibitory effect of parenterally administered compounds can be prolonged by slowing their absorption. One way to slow the absorption of a particular compound is by administering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is by administering injectable depot forms comprising the compound as an oleaginous solution or suspension. Yet another way to slow absorption of a particular compound is by administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents, and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefor.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of the present invention.

The total daily dose of the compounds administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Preferred compounds of the present invention include, but are not limited to: compounds of formula (I) wherein R¹ is a heterocycle selected from the group consisting of oxazolyl and dihydrooxazolyl. Preferably, the oxazolyl is 1,3-oxazol-2-yl; and the dihydrooxazolyl is 4,5-dihydro-1,3-oxazol-2-yl; and compounds of formula (I) wherein R¹ is aminocarbonyl. Most preferred compounds of the present invention include compounds of formula (I) wherein R is —C(O)NHCH₃.

Determination of Biological Activity

Activity Assay for Human Histone Deacetylases:

Nuclear histone deacetylase enzymes were partially purified from human erythroleukemia K562 cells by MonoQ sepharose chromatography (Proceedings of the National Academy of Sciences of the United States of America 1999, 96, 4592). The substrates, (³H)⁻labeled nuclear histones, were prepared from K562 cells by incubation of cells with (³H)⁻acetic acid in the presence of 3 mM trichostatin A and isolated by dounce homogenization, acid extraction of isolated nuclei, and acetone precipitation (J. Biol. Chem. 1990, 265, 17174). The standard assay consisted of 3-6 μg of histone deacetylase incubated with 5-10 μg (˜10,000 cpm) of labeled-nuclear histones for 1 hour at 37° C. in a 50 mL reaction volume. Inhibitor was added 15 minutes prior to substrate addition. The reaction was terminated by the addition of IM HCl/0.16M acetic acid (50 mL) and ethyl acetate (500 mL). The mixture was inverted for 30 seconds and the phases were separated by centrifugation (1000 rpm for 2 minutes). An aliquot of the organic phase was removed and counted in a liquid scintillation spectrophotometer. IC₅₀ values were determined by log-logit linear regression of the dose response data.

The compounds of the present invention were found to inhibit histone deacetylase with inhibitory potencies less than 50 μM. In a preferred range, the compounds inhibited histone deacetylase with inhibitory potencies less than 10 μM. In a more preferred range, the compounds inhibited histone deacetylase with inhibitory potencies less than 5 μM, and in a most preferred range, the compounds inhibited histone deacetylase with inhibitory potencies less than 1 μM. Thus, the compounds of the present are useful for treating diseases in which histone deacetylase plays a role.

Synthetic Methods

Abbreviations which have been used in the descriptions of the schemes and the examples that follow are: DMAP for 4-dimethylaminopyridine; CDI for 1,1′-carbonyldiimidazole; EDCI for 1-(3-dimethylaminopropyl)-3-ethylcarbodiimide hydrochloride; DCC for 1,3-dicyclohexylcarbodiimide; HOBt for 1-hydroxybenzotriazole hydrate; DMF for N,N-dimethylformamide; NMP for N-methylpyrrolidinone; THF for tetrahydrofuran; MTBE for methyl tert-butyl ether; DMSO for dimethylsulfoxide; OAc for acetate; DME for 1,2-dimethoxyethane; DEAD for diethyl azodicarboxylate; DIAD for diisopropyl azodicarboxylate; LAH for lithium aluminum hydride; NMM for N-methylmorpholine; TBAF for tetrabutylammonium fluoride; DBU for 1,8-diazabicyclo(5.4.0)undec-7-ene; pTsOH for p-toluenesulfonic acid; DBN for 1,5-diazabicyclo(4.3.0)non-5-ene; LDA for lithium diisopropylamide; KHMDS for potassium hexamethyldisilazide; PDC for pyridinium dichromate; NBS for N-bromosuccinimide; TBS for tert-butyldimethylsilyl, and mCPBA for m-chloroperoxybenzoic acid.

The compounds and processes of the present invention will be better understood in connection with the following synthetic schemes which illustrate the methods by which the compounds of the invention may be prepared. Starting materials can be obtained from commercial sources or prepared by well-established literature methods known to those of ordinary skill in the art. The groups L¹, L², R¹, R², R³, R⁴, R⁵, and R⁶ are as defined above unless otherwise noted below.

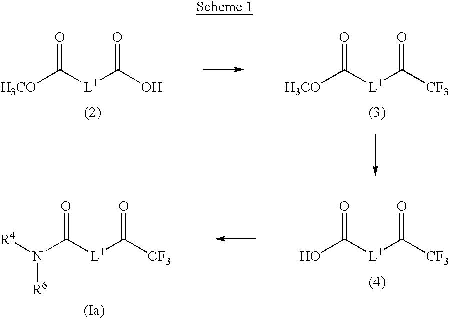

As shown in Scheme 1, compounds of formula (2) can be converted to compounds of formula (3) by treatment with a base and trifluoroacetic anhydride. Examples of bases used in these reactions include sodium hydride, lithium hexamethyldisilazide, pyridine, and mixtures thereof. Representative solvents used in these reactions include dichloromethane, carbon tetrachloride, and chloroform. The reaction is conducted at about −10° C. to about 5° C. and reaction times are typically about 2 to about 24 hours.

Compounds of formula (3) can be converted to compounds of formula (4) by hydrolysis methods known to those of ordinary skill in the art.

Conversion of compounds of formula (4) to compounds of formula (Ia) can be accomplished by treatment with an appropriately substituted amine (HNR⁴R⁶) in the presence of a base and a coupling agent. Examples of bases include NMM, DMAP, and triethylamine. Representative coupling agents include CDI, EDCI, DCC, HOBt, and mixtures thereof. Solvents typically used in these reactions include DMF, NMP, and dioxane. The reaction is conducted at about 20° C. to about 40° C. and reaction times are typically about 12 to about 24 hours.

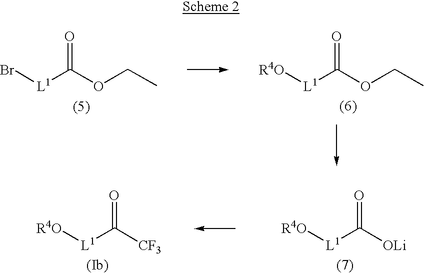

As shown in Scheme 2, compounds of formula (5) can be converted to compounds of formula (6) by treatment with an appropriately substituted alcohol (R⁴OH) in the presence of a base. Example of bases include Cs₂CO₃, K₂CO₃, and Na₂CO₃. Representative solvents include DMF, NMP, and dioxane. The reaction is conducted at about 20° C. to about 40° C. and reaction times are typically about 12 to about 24 hours.

Compounds of formula (6) can be converted to compounds of formula (7) by hydrolysis methods known to those of ordinary skill in the art.

Conversion of compounds of formula (7) to compounds of formula (Ib) can be accomplished by treatment with trifluoroacetic anhydride. Solvents commonly used in these reactions include dichloromethane, chloroform, and carbon tetrachloride. The reaction is conducted at about 20° C. to about 40° C. and reaction times are typically about 2 to about 4 hours.

Compounds of formula (Ib) wherein L¹ is alkynylene or alkenylene can be readily converted to compounds of formula (Ib) wherein L¹ is alkenylene or cycloalkylene, respectively, by methods such as cyclopropanation and reduction, well-known to those of ordinary skill in the art.

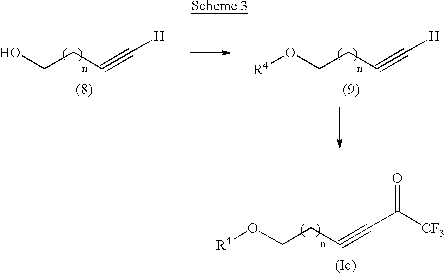

As shown in Scheme 3, compounds of formula (8) (n is a positive integer between 1 and 7) can be treated with an appropriately substituted alcohol (R⁴OH) in the presence of a trialkylphosphine or triarylphosphine and a diazo compound to provide compounds of formula (9). Representative trialkylphosphines include tributylphosphine and trimethylphosphine; representative triarylphosphines include triphenylphosphine and tri-o-tolylphosphine; and representative diazo compounds include DEAD and DIAD. Solvents commonly used in these reactions include THF, diethyl ether, and methyl tert-butyl ether. The reaction is conducted at about −5° C. to about 30° C., and typical reaction times are about 12 to about 24 hours.

Compounds of formula (9) can be converted to compounds of formula (Ic) by treatment with base followed by an ester of trifluoroacetic acid. Representative bases include n-butyllithium, tert-butyllithium, and lithium hexamethyldisilazide. Examples of solvents used in these reactions include THF, diethyl ether, and methyl tert-butyl ether. The reaction is conducted at about −78° C. to about 30° C. and typical reaction times are about 18 to about 24 hours.

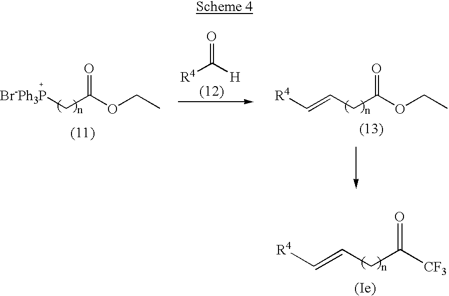

As shown in Scheme 4, compounds of formula (11) (n is a positive integer between 1 and 8) can be treated with compounds of formula (12) and base to provide compounds of formula (13). Examples of bases used in these reactions include potassium tert-butoxide and sodium tert-butoxide. Representative solvents include THF, methyl tert-butyl ether, and diethyl ether. The reaction temperature is about −5° C. to about 25° C. and reaction times are typically about 1 to about 3 hours.

Compounds of formula (13) can be converted to compounds of formula (Ie) following the procedures described in Scheme 2.

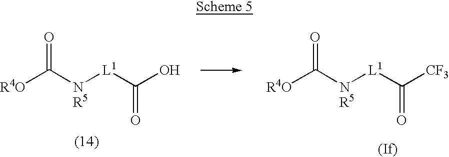

As shown in Scheme 5, compounds of formula (14) can be converted to compounds of formula (If) by treatment with oxalyl chloride, followed by treatment with trifluoroacetic anhydride and base. Examples of bases include pyridine, triethylamine, and diisopropylethylamine. Representative solvents include dichloromethane, 1,2-dichloroethane, and carbon tetrachloride. The reaction temperature is about −60° C. to about 25° C. and reaction times are typically about 2 hours to about 4 hours.

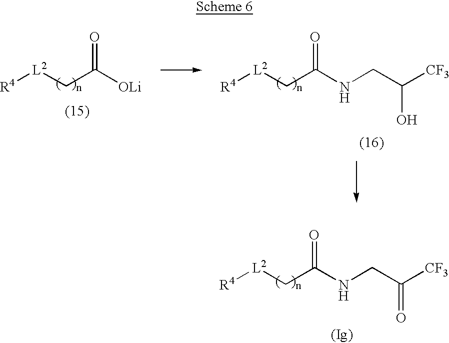

As shown in Scheme 6, compounds of formula (15) (prepared from the corresponding ester according to the procedures described in Scheme 2) can be converted to compounds of formula (16) by treatment with 2-hydroxy-2-(trifluoromethyl)ethylamine (prepared as described in J. Org. Chem. 1995, 60, 41) using the conditions described in Scheme 2.

Conversion of compounds of formula (16) to compounds of formula (Ig) can be accomplished by oxidation, using a variety of procedures known to those of ordinary skill in the art.

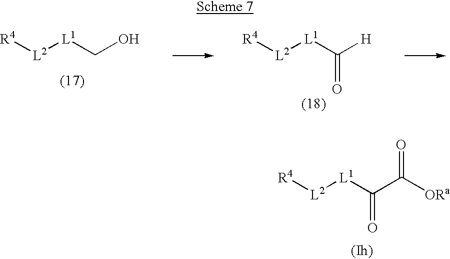

As shown in Scheme 7, compounds of formula (17) can be oxidized to compounds of formula (18) by numerous methods well-known to those of ordinary skill in the art, such as the Swern oxidation and the Dess-Martin oxidation.

Compounds of formula (18) can be converted to compounds of formula (Ih) (R^{a} is alkyl) by treatment with an alkyl ester of (dimethoxyphosphoryl)(tetrahydro-2H-pyran-2-yloxy)acetic acid (which can be prepared following the procedure described in Tet. Lett. 1981, 22, 663-666) in the presence of base. Representative bases include DBU, DBN, and DMAP. Examples of solvents used in these reactions include acetonitrile, THF, and diethyl ether. The reaction is conducted at about 0° C. to about 25° C. and reaction times are typically about 1 to about 3 hours.

Compounds of formula (Ih) where R^{a} is alkyl can be intraconverted to compounds of formula (Ih) where R^{a} is hydrogen by the hydrolysis methods shown in Scheme 1.

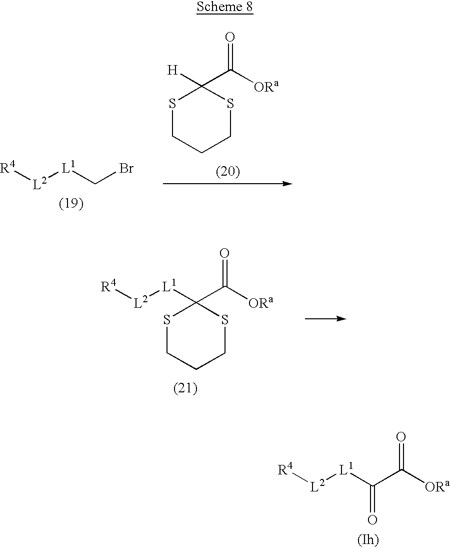

Scheme 8 shows an alternative synthesis of compounds of formula (Ih). Compounds of formula (19) can be reacted with compounds of formula (20) (R^{a} is alkyl) in the presence of base to provide compounds of formula (21). Representative bases include NaH, KH, and LiHMDS. Examples of solvents used in these reactions include DMF, THF, and diethyl ether. The reaction is conducted at about −78° C. to about 0° C. and reaction times are typically about 12 to about 24 hours.

Compounds of formula (21) can be converted to compounds of formula (Ih) by treatment with a variety of deprotection reagents such as NBS, known to those of ordinary skill in the art.

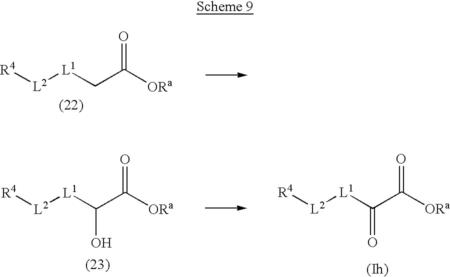

Another route to compounds of formula (Ih) is shown in Scheme 9. Compounds of formula (22) can be converted to compounds of formula (23) by treatment with 2-benzenesulfonyl-3-phenyl-oxaziridine (prepared according to the procedure described in J. Org. Chem. 1992, 47, 1774-1775) in the presence of base. Representative bases include KHMDS, LiHMDS, and LDA. Examples of solvents used in these reactions include THF, MTBE, and diethyl ether. The reaction is conducted at about −78° C. to about 0° C. and reaction times are typically about 30 minutes to about 2 hours.

Compounds of formula (23) can be converted to compounds of formula (Ih) by treatment with a variety of oxidation reagents, such as PDC, known to those of ordinary skill in the art.

Compounds of formula (Ih) can be reacted with various primary or secondary amines to form the corresponding ketoamides using procedures well-known to those of ordinary skill in the art.

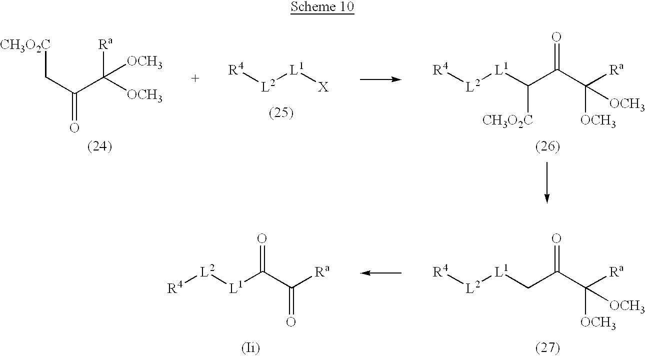

As shown in Scheme 10, compounds of formula (24) (R^{a} is alkyl) can be treated sequentially with a base and with compounds of formula (25) to provide compounds of formula (26). Representative bases include sodium hydride, potassium hydride, lithium hexamethyldisilazide, and lithium diisopropylamide. Examples of solvents used in these reactions include DMF, THF, MTBE, and diethyl ether. The reaction is typically conducted at about −78° C. to about 25° C. for about 2 to about 48 hours.

Compounds of formula (26) can be converted to compounds of formula (27) by treatment with a hydrolyzing agent. Representative hydrolyzing agents include sodium hydroxide and lithium hydroxide. Examples of solvents used in these reactions include methanol and ethanol. The reaction is conducted at about 25° C. to about 75° C. for about 1 to about 6 hours.

Conversion of compounds of formula (27) to compounds of formula (Ii) can be accomplished by treatment with a deprotecting agent such as HCl, trifluoroacetic acid, p-toluenesulfonic acid, or acetic acid. The reaction is conducted at about 0° C. to about 35° C. for about 1 to about 12 hours.

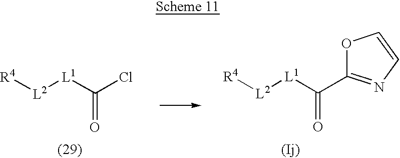

Scheme 11 shows the conversion of compounds of formula (29) to compounds of formula (Ij). Treatment of compounds of formula (29) with a stabilized anion of a heterocycle (generated by deprotonation with a strong base such as n-butyllithium at −78° C. followed by treatment with zinc chloride) in the presence of stoichiometric copper (such as copper iodide) gives compounds of formula (Ij). Examples of solvents used in these reactions include THF, diethyl ether, and MTBE. The reaction is conducted at about −78° C. to about 0° C. and reaction times are typically about 1 to about 3 hours.

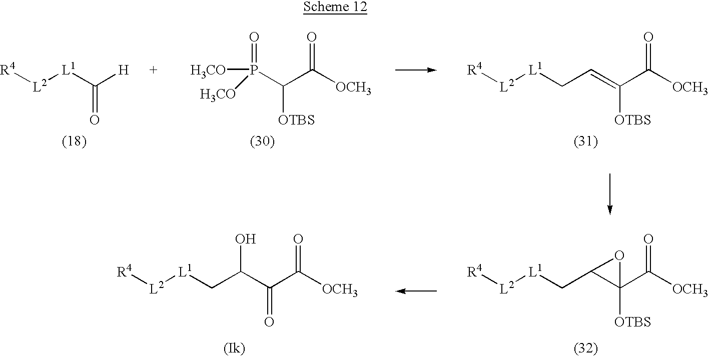

As shown in Scheme 12, compounds of formula (18) (prepared according to the methods described in Scheme 7) can be reacted with compounds of formula (30) in the presence of base and lithium chloride to provide compound of formula (31). Examples of bases include DBU, diisopropylethylamine, and sodium hydride. Representative solvents include THF, MTBE, and dioxane. The reaction is conducted at about 0 to about 23° C. for about 1 to about 16 hours.

Compounds of formula (31) can be reacted with an oxidizing agent to produce compounds of formula (32). Representative oxidizing agents include mCPBA with potassium fluoride, and t-butyl peroxide with n-butyllithium. Examples of solvents include dichloromethane, THF, and chloroform. The reaction is conducted at about 0 to about 23° C. for about 8 to about 16 hours.

Conversion of compounds of formula (32) to compounds of formula (Ik) can be accomplished by treatment with triethylamine trihydrofluoride. Examples of solvents used in this reaction include acetonitrile, tetrahydrofuran, and toluene. The reaction is conducted at about 0 to about 23° C. for about 8 to about 16 hours.

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects.

### Example 1

### 9,9,9-trifluoro-8-oxo-N-phenylnonanamide

### Example 1A

### methyl 9,9,9-trifluoro-8-oxononanoate

A suspension of NaH (750 mg, 29.7 mmol) in dichloromethane (150 mL) at 0° C. was treated dropwise with 8-methoxy-8-oxooctanoic acid (5.10 g, 27.1 mmol), stirred until gas evolution ceased, treated with trifluoroacetic anhydride (34.2 g, 163 mmol), stirred for 10 minutes, and treated with pyridine (18.9 g, 225 mmol). The mixture was warmed to room temperature, stirred for 1.5 hours, poured over ice (400 g), and warmed to room temperature. The layers were separated and the aqueous phase was extracted with dichloromethane. The combined extracts were dried (Na₂SO₄), filtered, and concentrated. The concentrate was purified by flash column chromatography on silica gel with 1: 1 dichloromethane/hexanes to provide 2.68 g (41%) of the desired product.

MS (ESI(−)) m/e 239 (M−H)⁻.

### Example 1B

### 9,9,9-trifluoro-8-oxononanoic acid

A solution of Example 1A (1.40 g, 5.8 mmol) in THF (25 mL) at room temperature was treated with 2M LiOH (35 mL, 70 mmol), stirred for 18 hours, and concentrated. The remaining solution was adjusted to pH 2 with IN HCl and extracted with ethyl acetate. The combined extracts were dried (Na₂SO₄), filtered, and concentrated to provide 1.28 g (98%) of the desired product of sufficient purity for subsequent use.

MS (ESI(−)) m/e 225 (M−H)⁻.

### Example 1C

### 9,9,9-trifluoro-8-oxo-N-phenylnonanamide

A solution of Example 1B (256 mg, 1.1 mmol), aniline (112 mg, 1.2 mmol), HOBt (179 mg, 1.3 mmol) and N-methylmorpholine (221 mg, 2.2 mmol) in DMF (3 mL) at room temperature was treated with EDCI (254 mg, 1.3 mmol), stirred for 18 hours, poured into water (50 mL), and extracted with ethyl acetate. The combined extracts were dried (Na₂SO₄), filtered, and concentrated. The concentrate was purified by flash column chromatography on silica gel with 7:3 hexanes/ethyl acetate to provide 273 mg (82%) of the desired product.

MS (ESI(+)) m/e 302 (M+H)⁺;

¹H NMR (300 MHz, DMSO-d₆) δ 9.83 (s, 1H), 7.58 (d, 2H), 7.35-7.30 (m, 2H), 7.01 (td, 1H), 2.86 (t, 2H), 2.29 (t, 2H), 1.65-1.50 (m, 4H), 1.47-1.23 (m, 4H);

Anal. Calcd for C₁₅H₁₈F₃NO₂: C, 59.79; H, 6.02; N, 4.65. Found: C, 59.62; H, 5.91; N, 4.51.

### Example 2

### 8-((1, ′-biphenyl)-4-yloxy)-1,1,1-trifluoro-2-octanone

### Example 2A

### ethyl 7-((1,1′-biphenyl)-4-yloxy)heptanoate

A mixture of ethyl 7-bromoheptanoate (53.1 g, 15.4 mmol), (1,1′-biphenyl)-4-ol (2.61 g, 15.3 mmol), and Cs₂CO₃ (5.49 g, 16.9 mmol) in DMF (50 mL) at room temperature was stirred for 18 hours, poured into ice water (400 mL), and filtered to provide 4.87 g (98%) of the desired product.

MS (ESI(+)) m/e 327 (M+H)⁺.

### Example 2B

### lithium 7-((1,1′-biphenyl)-4-yloxy)heptanoate

A solution of Example 2A (4.86 g, 14.9 mmol) in THF (15 mL) at room temperature was treated with 2M LiOH (25 mL, 50 mmol), heated to 80° C. for 2 hours, cooled to room temperature, filtered, and dried to provide 4.30g (95%) of the desired product.

MS (ESI(−)) m/e 297 (M−Li)⁻.

### Example 2C

### 8-((1,1′-biphenyl)-4-yloxy)-111-trifluoro-2-octanone

A solution of trifluoroacetic anhydride (2.14 g, 10.2 mmol) in dichloromethane (16 mL) at room temperature was treated with Example 2B (502 mg, 1.66 mmol), and pyridine (1.12 g, 13.4 mmol), stirred for 3 hours, and quenched with water (5 mL). The mixture was stirred for 10 minutes, poured into water (75 mL), and extracted with dichloromethane. The combined extracts were dried (Na₂SO₄), filtered, and concentrated. The concentrate was purified by flash column chromatography on silica gel with dichloromethane to provide 235 mg (40%) of the desired product.

MS (ESI(−)) m/e 349 (M−H)⁻;

¹H NMR (300 MHz, DMSO-d₆) δ 7.62-7.56 (m, 4H), 7.45-7.40 (m, 2H), 7.37-7.33 (m, 1H), 7.00 (d, 2H), 4.00 (t, 2H), 2.89 (t, 2H), 1.75-1.68 (m, 2H), 1.65-1.55 (m, 2H), 1.47-1.36 (m, 4H);

Anal. Calcd for C₂₀H₂₁F₃O₂: C, 68.56; H, 6.04. Found: C, 68.35; H, 6.10.

### Example 3

### 4′-((8,8,8-trifluoro-7-oxooctyl)oxy)(, 1′-biphenyl)-4-carbonitrile

The desired product was prepared by substituting 4′-hydroxy(1,1′-biphenyl)-4-carbonitrile for (1,1′-biphenyl)-4-ol in Example 2.

MS (ESI(−)) m/e 374 (M−H)⁻;

¹H NMR (300 MHz, DMSO-d₆) δ 7.87 (d, 2H), 7.83 (d, 2H), 7.70 (d, 2H), 7.05 (d, 2H), 4.03 (t, 2H), 2.89 (t, 2H), 1.76-1.69 (m, 2H), 1.62-1.57 (m, 2H), 1.49-1.36 (m, 4H);

Anal. Calcd for C₂₁H₂₀F₃NO₂: C, 67.19; H, 5.37; N, 3.73. Found: C, 67.24; H, 5.29; N, 3.58.

### Example 4

### 9-((1,1′-biphenyl)-4-yloxy)-1,1,1-trifluoro-2-nonanone

The desired product was prepared by substituting ethyl 8-bromooctanoate for ethyl 7-bromoheptanoate in Example 2.

MS (ESI(−)) m/e 363 (M−H)⁻;

¹H NMR (300 MHz, DMSO-d₆) δ 7.62-7.55 (m, 4H), 7.45-7.39 (m, 2H), 7.33-7.27 (m, 1H), 7.01 (d, 2H), 4.00 (t, 2H), 2.88 (t, 2H), 1.75-1.68 (m, 2H), 1.60-1.55 (m, 2H), 1.43-1.32 (m, 6H);

Anal. Calcd for C₂₁H₂₃F₃O₂: C, 69.22; H, 6.36. Found: C, 69.12; H, 6.28.

### Example 5

### 7-((1,1′-biphenyl)-4-yloxy)-1,1,1-trifluoro-2-heptanone

The desired product was prepared by substituting ethyl 6-bromohexanoate for ethyl 7-bromoheptanoate in Example 2.

MS (ESI(−)) m/e 335 (M−H)⁻;

¹H NMR (300 MHz, DMSO-d₆) δ 7.62-7.57 (m, 4H), 7.45-7.40 (m, 2H), 7.33-7.27 (m, 1H), 7.01 (d, 2H), 4.01 (t, 2H), 2.92 (t, 2H), 1.78-1.71 (m, 2H), 1.71-1.62 (m, 2H), 1.51-1.43 (m, 2H);

Anal. Calcd for C₁₉H₁₉F₃O₂: C, 67.85; H, 5.69. Found: C, 67.82; H, 5.69.

### Example 6

### 9,9,9-trifluoro-8-oxo-N-(4-pyridinyl)nonanamide

A mixture of Example 1B (50 mg, 0.22 mmol), HOBt (30 mg, 0.22 mmol), carbodiimide PS resin (720 mg), and 4-aminopyridine (25 mg, 0.27 mmol) in DMF (5 mL) at room temperature was agitated in a Quest 210 parallel synthesizer for 18 hours, treated with trisamine PS resin (220 mg), and agitated for 2 hours. The solution was decanted, the resin was rinsed with dichloromethane, and the combined solutions were concentrated. The concentrate was purified by preparative HPLC with a gradient system of 0 to 95% over 10 min of CH₃CN (containing 0.1%TFA) in water to provide the desired product.

MS (ESI(+)) m/e 303 (M+H)⁺;

¹H NMR (300 MHz, DMSO-d₆) δ 11.11 (s, 1H), 8.64 (d, 2H), 7.96 (d, 2H), 2.87 (t, 2H), 2.46 (t, 2H), 1.63-1.55 (m, 4H), 1.34-1.29 (m, 4H);

Anal. Calcd for C₁₆H₂₀F₃NO₂.CF₃CO₂H.0.1H₂O: C, 45.96; H, 4.39; N, 6.70. Found: C, 45.60; H, 4.30; N, 6.70.

### Example 7

### N-benzyl-9,9,9-trifluoro-8-oxononanamide

The desired product was prepared by substituting benzylamine for 4-aminopyridine in Example 6.

MS (ESI(+)) m/e 316 (M+H)⁺;

¹H NMR (300 MHz, DMSO-d₆) δ 8.32-8.25 (m, 1H), 7.33-7.28 (m, 2H), 7.26-7.20 (m, 3H), 4.25 (d, 2H), 2.85 (t, 2H), 2.13 (t, 2H), 1.64-1.46 (m, 4H), 1.33-1.28 (m, 4H);

Anal. Calcd for C₁₆H₂₀F₃NO₂.0.75H₂O: C, 58.44; H, 6.59; N, 4.26. Found: C, 58.18; H, 6.45; N, 4.05.

### Example 8

### 9,9,9-trifluoro-8-oxo-N-(3-pyridinylmethyl)nonanamide

The desired product was prepared by substituting 3-pyridinylmethanamine for 4-aminopyridine in Example 6.

MS (ESI(+)) m/e 317 (M+H)⁺;¹H NMR (300 MHz, DMSO-d₆) δ 8.61-8.58 (m, 2H), 8.44-8.40 (m, 1H), 7.97-7.94 (m, 1H), 7.62 (dd, 1H), 4.34 (d, 2H), 2.85 (t, 2H), 2.14 (t, 2H), 1.62-1.51 (m, 4H), 1.28-1.22 (m, 4H).

### Example 9

### 9,9,9-trifluoro-8-oxo-N-(2-phenylethyl)nonanamide

The desired product was prepared by substituting 2-phenylethanamine for 4-aminopyridine in Example 6.

MS (ESI(+)) m/e 330 (M+H)⁺;

¹H NMR (300 MHz, DMSO-d₆) δ 7.84 (m, 1H), 7.31-7.26 (m, 2H), 7.21-7.18 (m, 3H), 3.25 (q, 2H), 2.85 (t, 2H), 2.69 (t, 2H), 2.02 (t, 2H), 1.58-1.43 (m, 4H), 1.30-1.18 (m, 4H).

### Example 10

### 9,9,9-trifluoro-N-(4-methoxyphenyl)-8-oxononanamide

The desired product was prepared by substituting 4-methoxyaniline for 4-aminopyridine in Example 6.

MS (ESI(+)) m/e 332 (M+H)⁺;

¹H NMR (300 MHz, DMSO-d₆) δ 9.68 (s, 1H), 7.47 (d, 2H), 6.85 (d, 2H), 3.71 (s, 3H), 2.88-2.84 (m, 2H), 2.28-2.23 (m, 2H), 1.64-1.52 (m, 4H), 1.34-1.25 (m, 4H);

Anal. Calcd for C₁₆H₂₀F₃NO₃.0.7H₂O: C, 55.87; H, 6.27; N, 4.07. Found: C, 55.64; H, 6.13; N, 3.88.

### Example 11

### 9,9,9-trifluoro-N-(3-methoxyphenyl)-8-oxononanamide

The desired product was prepared by substituting 3-methoxyaniline for 4-aminopyridine in Example 6.

MS (ESI(+)) m/e 332 (M+H)⁺;

¹H NMR (300 MHz, DMSO-d6) δ 9.84 (s, 1H), 7.30 (br s, 1H), 7.21-7.09 (m, 2H), 6.61-6.59 (m, 1H), 3.71 (s, 3H), 2.87 (t, 2H), 2.28 (t, 2H), 1.62-1.51 (m, 4H), 1.36-1.25 (m, 4H);

Anal. Calcd for: C₁₆H₂₀F₃NO₃.0.6H₂O: C, 56.17; H, 6.25; N, 4.09. Found: C, 55.81; H, 6.04; N, 3.91.

### Example 12

### 9,9,9-trifluoro-N-(2-methoxyphenyl)-8-oxononanamide

The desired product was prepared by substituting 2-methoxyaniline for 4-aminopyridine in Example 6.

MS (ESI(+)) m/e 332 (M+H)⁺;

¹H NMR (300 MHz, DMSO-d₆) δ 8.99 (s, 1H), 7.91 (d, 1H), 7.08-7.00 (m, 2H), 6.91-6.85 (m, 1H), 3.82 (s, 3H), 2.87 (t, 2H), 2.36 (t, 2H), 1.64-1.51 (m, 4H), 1.34-1.26 (m, 4H).

### Example 13

### 9,9,9-trifluoro-8-oxo-N-(3-phenylpropyl)nonanamide

The desired product was prepared by substituting 3-phenyl-1-propanamine for 4-aminopyridine in Example 6.

MS (ESI(+)) m/e 344 (M+H)⁺;

¹H NMR (300 MHz, DMSO-d₆) δ 7.78 (br t, 1H), 7.30-7.25 (m, 2H), 7.20-7.14 (m, 3H), 3.03 (dd, 2H), 2.85 (t, 2H), 2.58-2.53 (m, 2H), 2.05 (t, 2H), 1.72-1.43 (m, 6H), 1.31-1.21 (m, 4H);

Anal. Calcd for C₁₈H₂₄F₃NO₂.H₂O: C, 59.82; H, 7.25; N, 3.88. Found: C, 59.42; H, 6.94; N, 3.80.

### Example 14

### N-(4-(dimethylamino)phenyl)-9,9,9-trifluoro-8-oxononanamide

The desired product was prepared by substituting 4-dimethylaminoaniline for 4-aminopyridine in Example 6.

MS (ESI(+)) m/e 345 (M+H)⁺;

¹H NMR (300 MHz, DMSO-d₆) δ 9.68 (br s, 1H), 7.47 (br d, 2H), 6.95-6.83 (br s, 1H), 6.64-6.54 (br s, 1H), 2.92 (br s, 6H), 2.86 (t, 2H), 2.25 (t, 2H), 1.65-1.52 (m, 4H), 1.33-1.25 (m, 4H).

### Example 15

### N-(1,3-benzodioxol-5-yl)-9,9,9-trifluoro-8-oxononanamide

The desired product was prepared by substituting 1,3-benzodioxol-5-amine for 4-aminopyridine in Example 6.

MS (ESI(+)) m/e 346 (M+H)⁺;

¹H NMR (300 MHz, DMSO-d₆) δ 9.76 (br s, 1H), 7.31-7.30 (br s, 1H), 6.95-6.92 (m,1H), 6.82 (d, 1H), 5.96 (s, 2H), 2.87 (t, 2H), 2.25 (t, 2H), 1.62-1.52 (m, 4H), 1.35-1.26 (m, 4H);

Anal. Calcd for C₁₆H₁₈F₃NO₄: C, 55.65; H, 5.25; N, 4.06. Found: C, 55.18; H, 5.00; N, 4.51.

### Example 16

### 9-(1,4-dioxa-8-azaspiro(4.5)dec-8-yl)-1,1,1-trifluoro-9-oxo-2,2-nonanediol

The desired product was prepared by substituting 1,4-dioxa-8-azaspiro(4.5)decane for 4-aminopyridine in Example 6.

MS (ESI(+)) m/e 352 (M+H)⁺;

¹H NMR (300 MHz, DMSO-d₆) δ 6.65-6.48 (br s, 2H), 3.89 (s, 4H), 3.51-3.44 (m, 4H), 2.30 (t, 2H), 1.62-1.56 (m, 4H), 1.55-1.34 (m, 6H), 1.32-1.21 (m, 4H).

### Example 17

### N-(1,1′-biphenyl)-4-yl-9,9,9-trifluoro-8-oxononanamide

The desired product was prepared by substituting (1,1′-biphenyl)-4-amine for 4-aminopyridine in Example 6.

MS (ESI(+)) m/e 378 (M+H)⁺;

¹H NMR (300 MHz, DMSO-d₆) δ 9.96 (s, 1H), 7.70-7.59 (m, 6H), 7.46-7.42 (m, 2H), 7.35-7.30 (m, 1H), 2.88 (t, 2H), 2.35-2.30 (m, 2H), 1.67-1.52 (m, 4H), 1.38-1.29 (m, 4H);

Anal. Calcd for C₂₁H₂₂F₃NO₂.0.2H₂O: C, 66.20; H, 5.93; N, 3.68. Found: C, 66.06; H, 5.89; N, 3.67.

### Example 18

N-(1,1′-biphenyl)-3-vl-9,9,9-trifluoro-8-oxononanamide

The desired product was prepared by substituting (1,1′-biphenyl)-3-amine for 4-aminopyridine in Example 6.

MS (ESI(+)) m/e 378 (M+H)⁺;

¹H NMR (300 MHz, DMSO-d₆) δ 9.95 (s, 1H), 7.92 (s, 1H), 7.61-7.56 (m, 3H), 7.50-7.45 (m, 2H), 7.40-7.30 (m, 3H), 2.87 (t, 2H), 2.32 (t, 2H), 1.68-1.52 (m, 4H), 1.39-1.27 (m, 4H).

### Example 19

### N-(1, 1′-biphenyl)-2-yl-9,9,9-trifluoro-8-oxononanamide

The desired product was prepared by substituting (1,1′-biphenyl)-2-amine for 4-aminopyridine in Example 6.

MS (ESI(+)) m/e 378 (M+H)⁺;

¹H NMR (300 MHz, DMSO-d₆) δ 9.18 (s, 1H), 7.42-7.30 (m, 9H), 2.85 (t, 2H), 2.13-2.11 (m, 2H), 1.63-1.33 (m, 4H), 1.28-1.14 (m, 4H).

### Example 20

### N-(4-cyclohexylphenyl)-9,9,9-trifluoro-8-oxononanamide

The desired product was prepared by substituting 4-cyclohexylaniline for 4-aminopyridine in Example 6.

MS (ESI(+)) m/e 384 (M+H)⁺;

¹H NMR (300 MHz, DMSO-d₆) δ 9.74 (s, 1H), 7.46 (d, 2H), 7.11 (d, 2H), 2.86 (t, 2H), 2.49-2.38 (m, 1H), 2.26 (t, 2H), 1.82-1.65 (m, 5H), 1.64-1.51 (m, 4H), 1.41-1.25 (m, 9H).

Anal. Calcd for C₂₁₂₈F₃NO₂.0.3H₂O: C, 64.86; H, 7.41; N, 3.60. Found: C, 64.69; H, 7.16; N, 3.36.

### Example 21

### 9,9,9-trifluoro-8-oxo-N-(4-(1-piperidinyl)phenyl)nonanamide4

The desired product was prepared by substituting 4-(1-piperidinyl)aniline for 4-aminopyridine in Example 6.

MS (ESI(+)) m/e 385 (M+H)⁺;

¹H NMR (300 MHz, DMSO-d₆) δ 10.14-9.84 (br s, 1H), 7.68-7.55 (br s, 2H), 6.66-6.56 (br s, 2H), 2.87 (t, 2H), 2.51 (br s, 4H), 2.33-2.26 (m, 2H), 1.84-1.71 (m, 4H), 1.64-1.52 (m, 5H), 1.49-1.37 (m, 1H), 1.33-1.25 (m, 4H).

### Example 22

### 9,9,9-trifluoro-N-(4-(4-morpholinyl)phenyl)-8-oxononanamide

The desired product was prepared by substituting 4-(4-morpholinyl)aniline for 4-aminopyridine in Example 6.

MS (ESI(+)) m/e 387 (M+H)⁺;

¹H NMR (300 MHz, DMSO-d₆) δ 9.66 (s, 1H), 7.45 (d, 2H), 6.19 (d, 2H), 3.75-3.72 (m, 4H), 3.17-3.04 (m, 4H), 2.86 (t, 2H), 2.25 (t, 2H), 1.63-1.50 (m, 4H), 1.36-1.24 (m, 4H).

### Example 23

### N-((1S)-1-benzyl-2-(methylamino)-2-oxoethyl)-9,9,9-trifluoro-8-oxononanamide

The desired product was prepared by substituting (2S)-2-amino-N-methyl-3-phenylpropanamide for 4-aminopyridine in Example 6.

MS (ESI(+)) m/e 387 (M+H)⁺;

¹H NMR (300 MHz, DMSO-d₆) δ 8.03-7.98 (m, 1H), 7.92-7.84 (m, 1H), 7.31-7.13 (m, 5H), 4.49-4.38 (m, 1H), 2.99-2.93 (m, 1H), 2.82 (t, 2H), 2.71 (dd, 1H), 2.56 (d, 3H), 2.02 (t, 2H), 1.55-1.44 (m, 2H), 1.36-1.24 (m, 2H), 1.24-1.11 (m, 2H), 1.11-1.06 (m, 2H);

Anal. Calcd for C₁₉H₂₅F₃N₂O₃.0.1H₂O: C, 58.78; H, 6.54; N, 7.22. Found: C, 58.40; H, 6.57; N, 7.24.

### Example 24

### N-benzhydryl-9,9,9-trifluoro-8-oxononanamide

The desired product was prepared by substituting benzhydrylamine for 4-aminopyridine in Example 6.

MS (ESI(+)) m/e 392 (M+H)⁺;

¹H NMR (300 MHz, DMSO-d₆) δ 8.70 (d, 1H), 7.35-7.20 (m, 10H), 6.11 (d, 1H), 2.83 (t, 2H), 2.20 (t, 2H), 1.58-1.49 (m, 4H), 1.32-1.21 (m, 4H);

Anal. Calcd for C₂₂H₂₄F₃NO₂: C, 67.51; H, 6.18; N, 3.58. Found: C, 67.22; H, 6.13; N, 3.57.

### Example 25

### 9,9,9-trifluoro-8-oxo-N-(3-pyridinyl)nonanamide

The desired product was prepared by substituting 3-aminopyridine for 4-aminopyridine in Example 6.

MS (ESI(+)) m/e 321 (M+H₂O+H)⁺;

¹H NMR (300 MHz, DMSO-d₆) δ 10.13 (br s, 1H), 8.77-8.76 (m, 1H), 8.27-8.25 (m, 1H), 8.08-8.04 (m, 1H), 7.40-7.36 (m, 1H), 2.87 (t, 2H), 2.34 (t, 2H), 1.65-1.56 (m, 4H), 1.36-1.29 (m, 4H).

### Example 26

### N-cyclohexyl-9,9,9-trifluoro-8,8-dihydroxynonanamide

The desired product was prepared by substituting cyclohexylamine for 4-aminopyridine in Example 6.

MS (ESI(+)) m/e 308 (M+H)⁺;

¹H NMR (300 MHz, DMSO-d₆) δ 7.61 (d, 1H), 6.60 (s, 2H), 3.56-3.43 (m, 1H), 2.01 (t, 2H), 1.74-1.36 (m, 12H), 1.34-1.03 (m, 8H);

Anal. Calcd for C₁₅H₂₄F₃NO₂.0.9H₂O: C, 55.68; H, 8.04; N, 4.33. Found: C, 55.60; H, 8.19; N, 4.41.

### Example 27

### 9,9,9-trifluoro-N— (4-hydroxyphenyl)-8-oxononanamide

The desired product was prepared by substituting 4-hydroxyaniline for 4-aminopyridine in Example 6.

MS (ESI(+)) m/e 318 (M+H)⁺;

¹H NMR (300 MHz, DMSO-d₆) δ 9.56 (s, 1H), 9.11 (s, 1H), 7.34 (d, 2H), 6.64 (d, 2H), 2.86 (t, 2H), 2.23 (t, 2H), 1.62-1.52 (m, 4H), 1.34-1.26 (m, 4H);

Anal. Calcd for C₁₅H₁₈F₃NO₃: C, 56.78; H, 5.72; N, 4.41. Found: C, 56.73; H, 5.94; N, 4.38.

### Example 28

### 9,9,9-trifluoro-N-(4-fluorophenyl)-8-oxononanamide

The desired product was prepared by substituting 4-fluoroaniline for 4-aminopyridine in Example 6.

MS (ESI(+)) m/e 320 (M+H)⁺;

¹H NMR (300 MHz, DMSO-d₆) δ 9.91 (s, 1H), 7.59 (dd, 2H), 7.12 (t, 2H), 2.87 (t, 2H), 2.28 (t, 2H), 1.65-1.52 (m, 4H), 1.36-1.26 (m, 4H);

Anal. Calcd for C₁₅H₁₇F₄NO₂: C, 56.43; H, 5.37; N, 4.39. Found: C, 56.73; H, 5.94; N, 4.38.

### Example 29

### N-(3-cyanophenyl)-9,9,9-trifluoro-8-oxononanamide

The desired product was prepared by substituting 3-cyanoaniline for 4-aminopyridine in Example 6.

MS (ESI(−)) m/e 325 (M−H)⁻;

¹H NMR (300 MHz, DMSO-d₆) δ 10.23 (s, 1H), 8.11-8.09 (m, 1H), 7.78 (dt, 1H), 7.54-7.47 (m, 2H), 2.87 (t, 2H), 2.33 (t, 2H), 1.66-1.53 (m, 4H), 1.34-1.27 (m, 4H);

Anal. Calcd for C₁₆H₁₇F₃N₂O₂.0.5H₂O: C, 58.89; H, 5.25; N, 8.58. Found: C, 57.64; H, 5.29; N, 7.91.

### Example 30

N-(4-chlorophenyl)-9,9,9-trifluoro-8-oxononanamide

The desired product was prepared by substituting 4-chloroaniline for 4-aminopyridine in Example 6.

MS (ESI(+)) m/e 336 (M+H)⁺;

¹H NMR (300 MHz, DMSO-d₆) δ 9.98 (s, 1H), 7.61 (d, 2H), 7.33 (d, 2H), 2.89-2.84 (m, 2H), 2.30 (t, 2H), 1.64-1.53 (m, 4H), 1.35-1.24 (m, 4H);

Anal. Calcd for C₁₅H₁₇CIF₃NO₂.0.9H₂O: C, 51.19; H, 5.38; N, 3.98. Found: C, 51.07; H, 5.44; N, 3.99.

### Example 31

### N-(4-acetylphenyl)-9,9,9-trifluoro-8-oxononanamide

The desired product was prepared by substituting 1-(4-aminophenyl)ethanone for 4-aminopyridine in Example 6. MS (APCI(+)) 344 (M+H)⁺;

¹H NMR (300 MHz, DMSO-d₆) δ 10.21 (s, 1H), 7.91 (d, 2H), 7.71 (d, 2H), 2.87 (t, 2H,), 2.50 (s, 3H), 2.35 (t, 2H), 1.63-1.54 (m, 4H), 1.34-1.28 (m, 4H).

### Example 32

### N-(2-adamantyl)-9,9,9-trifluoro-8-oxononanamide

The desired product was prepared by substituting 2-adamantanamine for 4-aminopyridine in Example 6.

MS (ESI(+)) m/e 360 (M+H)⁺;

¹H NMR (300 MHz, DMSO-d₆) δ 7.60 (d, 1H), 3.82 (d, 1H), 2.85 (t, 2H), 2.13 (d, 2H), 1.99-1.94 (m, 2H), 1.84-1.67 (m, 1OH), 1.59-1.43 (m, 6H), 1.34-1.23 (m, 4H);

Anal. Calcd for C₁₉H₂₈F₃NO₂.0.1CF₃COOH: C, 62.19; H, 7.64; N, 3.78. Found: C, 62.64; H, 7.01; N, 3.44.

### Example 33

### 9,9,9-trifluoro-8-oxo-N-(4-(trifluoromethyl)phenyl)nonanamide

The desired product was prepared by substituting 4-trifluoromethylaniline for 4-aminopyridine in Example 6.

MS (ESI(+)) m/e 370 (M+H)⁺;

¹H NMR (300 MHz, DMSO-d₆) δ 10.23 (s, 1H), 7.79 (d, 2H), 7.65 (d, 2H), 2.87 (t, 2H), 2.34 (t, 2H), 1.64-1.57 (m, 4H), 1.35-1.29 (m, 4H).

### Example 34

### N-(3,4-dichlorophenyl)-9,9,9-trifluoro-8-oxononanamide

The desired product was prepared by substituting 3,4-dichloroaniline for 4-aminopyridine in Example 6.

MS (ESI(+)) m/e 370 (M+H)⁺;

¹H NMR (300 MHz, DMSO-d₆) δ 10.15 (s, 1H), 7.99 (d, 1H), 7.54 (d, 1H), 7.48-7.45 (dd, 1H), 2.86 (t, 2H), 2.31 (t, 2H), 1.64-1.52 (m, 4H), 1.34-1.28 (m, 4H).

### Example 35

### N-(4-bromophenyl)-9,9,9-trifluoro-8-oxononanamide

The desired product was prepared by substituting 4-bromoaniline for 4-aminopyridine in Example 6.

MS (ESI(+)) m/e 380 (M+H)⁺;

¹H NMR (300 MHz, DMSO-d₆) δ 10.00 (s, 1H), 7.56 (d, 2H), 7.46 (d, 2H), 2.87 (t, 2H), 2.30 (t, 2H), 1.64-1.53 (m, 4H), 1.34-1.26 (m, 4H);

Anal. Calcd for C₁₅Hl₇BrF₃NO₂.0.9H₂O: C, 45.45; H, 4.78; N, 3.53. Found: C, 45.37; H, 4.54; N, 3.38.

### Example 36

### N-(4-benzylphenyl)-9,9,9-trifluoro-8-oxononanamide

The desired product was prepared by substituting 4-benzylaniline for 4-aminopyridine in Example 6.

MS (ESI(+)) m/e 392 (M+H)⁺;

¹H NMR (300 MHz, DMSO-d₆) δ 9.77 (s, 1H), 7.48 (d, 2H), 7.30-7.25 (m, 2H), 7.21-7.17 (m, 3H), 7.12 (d, 2H), 3.87 (s, 2H), 2.87 (t, 2H), 2.26 (t, 2H), 1.63-1.50 (m, 4H), 1.34-1.26 (m, 4H);

Anal. Calcd for C₂₂H₂₄F₃NO₂: C, 67.51; H, 6.18; N, 3.58. Found: C, 67.36; H, 5.90; N, 3.44.

### Example 37

### 9,9,9-trifluoro-8-oxo-N-(4-phenoxyphenyl)nonanamide

The desired product was prepared by substituting 4-phenoxyaniline for 4-aminopyridine in Example 6.

MS (ESI(+)) m/e 394 (M+H)⁺;

¹H NMR (300 MHz, DMSO-d₆) δ 9.87 (s, 1H), 7.59 (d, 2H), 7.35 (dd, 2H), 7.12-7.06 (m, 1H), 6.99-6.59 (m, 4H), 2.87 (t, 2H), 2.29 (t, 2H), 1.66-1.52 (m, 4H), 1.48-1.26 (m, 4H);

Anal. Calcd for C₂₁H₂₂F₃NO₃: C, 64.11; H, 5.64; N, 3.56. Found: C, 64.01; H, 5.61; N, 3.51.

### Example 38

### 9,9,9-trifluoro-8,8-dihydroxy-N-(9-oxo-9H-fluoren-2-yl)nonanamide

The desired product was prepared by substituting 2-amino-9H-fluoren-9-one for 4-aminopyridine in Example 6.

MS (ESI(+)) m/e 404 (M+H)⁺;

¹H NMR (300 MHz, DMSO-d₆) δ 10.14 (s, 1H), 7.95 (br s, 1H), 7.71-7.68 (m, 3H), 7.60-7.55 (m, 2H), 7.43-7.28 (m, 1H), 6.61 (s, 2H), 2.33 (t, 2H), 1.65-1.59 (m, 4H), 1.49-1.39 (m, 2H), 1.36-1.28 (m, 4H);

Anal. Calcd for C₂₂H₂₀F₃NO₃.0.7H₂O: C, 63.52; H, 5.18; N, 3.37. Found: C, 63.24; H, 4.77; N, 3.28.

### Example 39

### N-(4-(benzyloxy)phenyl)-9,9,9-trifluoro-8-oxononanamide

The desired product was prepared by substituting 4-(benzyloxy)aniline for 4-aminopyridine in Example 6.

MS (ESI(+)) m/e 408 (M+H)⁺;

¹H NMR (300 MHz, DMSO-d₆) δ 9.70 (s, 1H), 7.49-7.32 (m, 7H), 6.93 (d, 2H), 5.05 (s, 2H), 2.86 (t, 2H), 2.25 (t, 2H), 1.63-1.52 (m, 4H), 1.34-1.28 (m, 4H).

### Example 40

### 9,9,9-trifluoro-N-(3-methoxypropyl)-8-oxononanamide

The desired product was prepared by substituting 3-methoxy-1-propanamine for 4-aminopyridine in Example 6.

MS (ESI(+)) m/e 298 (M+H)⁺.

### Example 41

### 9,9,9-trifluoro-N-isopentyl-8-oxononanamide

The desired product was prepared by substituting 3-methyl-1-butanamine for 4-aminopyridine in Example 6.

MS (ESI(+)) m/e 296 (M+H)⁺;

¹H NMR (300 MHz, DMSO-d₆) δ 7.68 (br m, 1H), 3.07-3.00 (m, 2H), 2.85 (t, 2H), 2.02 (t, 2H), 1.62-1.36 (m, 5H), 1.30-1.20 (m, 6H), 0.85 (d, 6H).

### Example 42

### N-(4′-cyano(1,1′-biphenyl)-3-yl)-9,9,9-trifluoro-8-oxononanarnide

The desired product was prepared by substituting 3′-amino(1,1′-biphenyl)-4-carbonitrile for 4-aminopyridine in Example 6.

MS (ESI(+)) m/e 403 (M+H);

¹H NMR (300 MHz, DMSO-d6) δ 10.02 (s, 1H), 8.01 (s, 1H), 7.94 (d, 2H), 7.81 (d, 2H), 7.61 (app d, 1H), 7.46-7.37 (m, 2H), 2.87 (t, 2H), 2.33 (t, 2H), 1.60-1.55 (m, 4H), 1.34 (m, 4H);

Anal. Calcd for C₂₂H₂₁F₃N₂O₂: C, 65.66; H, 5.26; N, 6.96. Found: C, 65.50; H, 5.37; N, 7.04.

### Example 43

### N-(3-(benzyloxy)phenyl)-9,9,9-trifluoro-8-oxononanamide

The desired product was prepared by substituting 3-(benzyloxy)aniline for 4-aminopyridine in Example 6.

MS (ESI(+)) m/e 408 (M+H)⁺;

¹H NMR (300 MHz, DMSO-d₆) δ 9.73 (s, 1H), 7.48-7.26 (m, 6H), 7.21-7.07 (m, 2H), 6.70-6.65 (m, 1H), 5.06 (s, 2H), 2.86 (t, 2H), 2.28 (t, 2H), 1.59-1.55 (m, 4H), 1.33-1.28 (m, 4H);

Anal. Calcd for C₂₂H₂₄F₃NO₃: C, 64.86; H, 5.94; N, 3.44. Found: C, 65.06; H, 5.95; N, 3.53.

### Example 44

### 9,9,9-trifluoro-8-oxo-N-(3-phenoxyphenyl)nonanamide

The desired product was prepared by substituting 3-phenoxyaniline for 4-aminopyridine in Example 6.

MS (ESI(+)) m/e 394 (M+H)⁺;

¹H NMR (300 MHz, DMSO-d₆) δ 9.92 (s, 1H), 7.42-7.27 (m, 5H), 7.15 (t, 1H), 7.02 (d, 2H), 6.67 (dt, 1H), 2.86 (t, 2H), 2.26 (t, 2H), 1.62-1.50 (m, 4H), 1.34-1.24 (m, 4H);

Anal. Calcd for C₂₁H₂₂F₃NO₃.0.2H₂O: C, 63.53; H, 5.69; N, 3.53. Found: C, 63.42; H, 5.62; N, 3.33.

### Example 45

### N-(3-benzoylphenyl)-9,9,9-trifluoro-8-oxononanamide

The desired product was prepared by substituting (3-aminophenyl)(phenyl)methanone for 4-aminopyridine in Example 6.

MS (ESI(−)) m/e 404 (M−H)⁻;

¹H NMR (300 MHz, DMSO-d₆) δ 10.10 (s, 1H), 8.01 (s, 1H), 7.90 (dt, 1H), 7.75-7.66 (m, 3H), 7.60-7.54 (m, 2H), 7.48 (t, 1H), 7.39 (d, 1H), 2.86 (t, 2H), 2.31 (t, 2H), 1.63-1.52 (m, 4H), 1.34-1.25 (m, 4H);

Anal. Calcd for C₂₂H₂₂F₃NO₃.0.2H₂O: C, 64.60; H, 5.52; N, 3.42. Found: C, 64.46; H, 5.34; N, 3.47.

### Example 46

### 9,9,9-trifluoro-8-oxo-N-(4-phenyl-1,3-thiazol-2-yl)nonanamide

The desired product was prepared by substituting 4-phenyl-1,3-thiazol-2-amine for 4-aminopyridine in Example 6.

MS (ESI(+)) m/e 385 (M+H)⁺;

¹H NMR (300 MHz, DMSO-d₆) δ 12.21 (s, 1H), 7.89 (d, 2H), 7.59 (s, 1H), 7.45-7.40 (m, 2H), 7.35-7.29 (m, 1H), 2.87 (t, 2H), 2.47 (t, 2H), 1.64-1.54 (m, 4H), 1.35-1.29 (m, 4H);

Anal. Calcd for C₁₈H₁₉F₃N₂O₂S: C, 56.24; H, 4.98; N, 7.29. Found: C, 55.99; H, 4.94; N, 6.96.

### Example 47

### 8-(3-bromophenoxy)-1,1,1-trifluoro-2-octanone

The desired product was prepared by substituting 3-bromophenol for (1,1′-biphenyl)-4-ol in Example 2.

MS (ESI(−)) m/e 351 (M−H)⁻;

¹H NMR (300 MHz, DMSO-d₆) δ 7.23 (t, 1H), 7.13-7.08 (m, 2H), 6.96-6.92 (m, 1H), 3.97 (t, 2H), 2.88 (t, 2H), 1.74-1.65 (m, 2H), 1.63-1.54 (m, 2H), 1.46-1.29 (m, 4H);

Anal. Calcd for C₁₄H₁₆BrF₃O₂.0.2H₂O:C, 47.13; H, 4.63. Found: C, 46.75; H, 4.75.

### Example 48

### 1,1,1-trifluoro-8-(3-(4-pyridinyl)phenoxy)-2-octanone

The desired product was prepared by substituting 3-(4-pyridinyl)phenol for (1,1′-biphenyl)-4-ol in Example 2.

MS (ESI(+)) m/e 352 (M+H)⁺;

¹H NMR (300 MHz, DMSO-d₆) δ 8.62 (d, 2H), 7.71 (d, 2H), 7.42 (t, 1H), 7.36-7.31 (m, 2H), 7.05-7.02 (m, 1H), 4.06 (t, 2H), 2.89 (t, 2H), 1.77-1.60 (m, 2H), 1.68-1.58 (m, 2H), 1.49-1.30 (m, 4H);

Anal. Calcd for C₁₉H₂₀F₃NO₂.HCl.2.3H₂O: C, 53.16; H, 6.01; N, 3.26. Found: C, 52.80; H, 5.99; N, 3.03.

### Example 49

### 8-(4-bromophenoxy)-1,1,1-trifluoro-2-octanone

The desired product was prepared by substituting 4-bromphenol for (1,1′-biphenyl)-4-ol in Example 2.

MS (ESI(−)) m/e 351 (M−H)⁻;

¹H NMR (300 MHz, DMSO-d₆) δ 7.42 (d, 2H), 6.89 (d, 2H), 3.94 (t, 2H), 2.87 (t, 2H), 1.72-1.65 (m, 2H), 1.62-1.54 (m, 2H), 1.46-1.30 (m, 4H);

Anal. Calcd for C₁₄H₁₆BrF₃O₂: C, 47.61; H, 4.57. Found: C, 47.88; H, 4.39.

### Example 50

### 1,1,1-trifluoro-8-(4-phenoxyphenoxy)-2-octanone

The desired product was prepared by substituting 4-phenoxyphenol for (1,1′-biphenyl)-4-ol in Example 2.

MS (ESI(−)) m/e 365 (M−H)⁻;

¹H NMR (300 MHz, DMSO-d₆) δ 7.36-7.32 (m, 2H), 7.09-7.03 (m, 1H), 6.99-6.88 (m, 6H), 3.94 (t, 2H), 2.88 (t, 2H), 1.73-1.64 (m, 2H), 1.61-1.54 (m, 2H), 1.40-1.35 (m, 4H);

Anal. Calcd for C₂₀H₂₁F₃O₃: C, 65.57; H, 5.78. Found: C, 65.26; H, 5.64.

### Example 51

### 8-((1,1′-biphenyl)-3-yloxy)-1,1,1-trifluoro-2-octanone

The desired product was prepared by substituting (1,1′-biphenyl)-3-ol for (1,1′-biphenyl)-4-ol in Example 2.

MS (ESI(−)) m/e 349 (M−H)⁻;

¹H NMR (300 MHz, DMSO-d6) δ 7.66 (d, 2H), 7.48-7.45 (m, 2H), 7.39-7.33 (m, 2H), 7.22-7.16 (m, 1H), 7.17-7.16 (m, 1H), 6.94-6.91 (m, 1H), 4.05-4.02 (m, 2H), 2.89 (t, 2H), 1.74 (m, 2H), 1.62-1.58 (m, 2H), 1.50-1.33 (m, 4H);

Anal. Calcd for C₂₀H₂₁F₃O₂: C, 68.56; H, 6.04;. Found: C, 68.64; H, 6.08.

### Example 52

### 9,9,9-trifluoro-8-oxo-N-(4′-(trifluoromethoxy)(1,1′-biphenyl)-3-yl)nonanamide

The desired product was prepared by substituting 4′-(trifluoromethoxy)(1,1′-biphenyl)-3-amine for aniline in Example 1.

MS (DCI) mle 462 (M+H)⁺;

¹H NMR (300 MHz, DMSO-d₆) δ 9.98 (s, 1H), 7.94 (s, 1H), 7.63 (d, 2H), 7.57 (d, 1H), 7.47 (d, 2H), 7.38 (d, 1H), 7.34 (d, 1H), 2.86 (t, 2H), 2.33 (t, 2H), 1.68-1.50 (m, 4H), 1.40-1.25 (m, 4H);

Anal. Calcd for C₂₂H₂₁NO₃F₆: C, 57.26; H, 4.58; N, 3.03. Found: C, 57.03; H, 4.65; N, 2.92.

### Example 53

### 9,9,9-trifluoro-8-oxo-N-(3-(3-pyridinyl)phenyl)nonanamide

The desired product was prepared by substituting 3-(3-pyridinyl)aniline for aniline in Example 1.

MS (DCI) m/e 379 (M+H)⁺;

¹H NMR (300 MHz, DMSO-d₆) δ 10.0 (s, 1H), 8.82 (s, 1H), 8.58 (d, 1H), 8.00 (d, 1H), 7.95 (s, 1H), 7.64 (d, 1H), 7.53-7.31 (m, 3H), 2.86 (t, 2H), 2.33 (t, 2H), 1.69-1.52 (m, 4H), 1.41-1.20 (m, 4H);

Anal. Calcd for C₂₀H₂₁N₂O₂F₃.0.5H₂O: C, 62.01; H, 5.72; N, 7.23. Found: C, 61.98; H, 5.73; N, 7.05.

### Example 54

### 9,9,9-trifluoro-N-(4′-(methylsulfanyl)(1,1′-biphenyl)-3-yl)-8-oxononanamide

### Example 54A

### N-(3-bromophenyl)-9,9,9-trifluoro-8-oxononanamide

The desired product was prepared by substituting 3-bromoaniline for aniline in Example 1.

MS (ESI(+)) m/e 381 (M+H)⁺.

### Example 54B

### 9,9,9-trifluoro-N-(4 ′-(methylsulfanyl)(1,1′-biphenyl)-3-yl)-8-oxononanamide

A mixture of Example 54A (308 mg, 0.81 mmol), 4-(methylsulfanyl)phenyl-boronic acid (150 mg, 0.89 mmol), Pd(OAc)₂ (9.1 mg, 0.04 mmol), tri-o-tolylphosphine (24.4 mg, 0.08 mmol), and 2M Na₂CO₃ (2 mL, 2 mmol) in DME (5 mL) was heated to 80° C. for 3 hours, treated with additional Pd (OAc)₂ (9 mg), tri-o-tolylphosphine (24 mg), and 3-(methylsulfanyl)phenyl-boronic acid (75 mg), heated for 3 hours, cooled to room temperature, and partitioned between diethyl ether and water. The aqueous phase was extracted with diethyl ether and the combined extracts were washed with brine, dried (Na₂SO₄), filtered, and concentrated. The concentrate was purified by flash column chromatography on silica gel with 7:3 hexanes/ethyl acetate to provide 164 mg (48%) of the desired product.

MS (ESI(−)) m/e 422 (M−H)⁻;

¹H NMR (300 MHz, DMSO-d₆) δ 9.94 (s, 1H), 7.90 (s, 1H), 7.55 (d, 3H), 7.42-7.22 (m, 4H), 2.86 (t, 2H), 2.49 (s, 3H), 2.33 (t, 2H), 1.68-1.51 (m, 4H), 1.36-1.27 (m, 4H);

Anal. Calcd for C₂₂H₂₄NO₂F₃S: C, 62.39; H, 5.71; N, 3.31. Found: C, 63.18; H, 5.60; N, 2.75.

### Example 55

### N-(3′-amino(1,1′-biphenyl)-3-yl)-9,9,9-trifluoro-8-oxononanamide

The desired product was prepared by substituting 3-aminophenylboronic acid for 4-(methylsulfanyl)phenylboronic acid in Example 54.

MS (ESI(−)) m/e 391 (M−H)⁻;

¹H NMR (300 MHz, DMSO-d₆) δ 9.92 (s, 1H), 7.87 (s, 1H), 7.50 (d, 1H), 7.20 (d, 1H), 7.09 (t, 1H), 6.78 (dd, 1H), 6.72 (d, 1H), 6.56 (d, 1H), 5.17 (s, 2H), 2.87 (t, 2H), 2.34 (t, 2H), 1.67-1.52 (m, 4H), 1.38-1.27 (m, 4H);

Anal. Calcd for C₂₁H₂₃N₂O₂F₃: C, 64.28; H, 5.91; N, 7.14. Found: C, 63.95; H, 5.99; N, 6.91.

### Example 56

### 9,9,9-trifluoro-N-(4′-(methylsulfonyl)(1,1′-biphenyl)-3-yl)-8-oxononanamide

A suspension of Example 54B (85 mg, 0.2 mmol) in a 2:1 mixture of methanol/water (10 mL) at room temperature was treated with NaHCO₃ (42 mg, 0.5 mmol) and oxone (10 mL), stirred for 18 hours, and partitioned between ethyl acetate and water. The aqueous phase was extracted with ethyl acetate and the combined extracts were washed with brine, dried (Na₂SO₄), filtered, and concentrated. The concentrate was recrystallized from ethyl acetate/hexane to provide 56 mg (62%) of the desired product.

MS (ESI(−)) m/e 454 (M−H)⁻;

¹H NMR (300 MHz, DMSO-d₆) δ 10.03 (s, 1H), 8.03 (d, 3H), 7.86 (d, 2H), 7.64 (d, 1H), 7.48-7.38 (m, 2H), 3.27 (s, 3H), 2.87 (t, 2H), 2.33 (t, 2H), 1.67-1.52 (m, 4H), 1.38-1.27 (m, 4H);

Anal. Calcd for C₂₂H₂₄NO4F₃.0.5H₂O: C, 56.89; H, 5.42; N, 3.02. Found: C, 56.80; H, 5.47; N, 2.82.

### Example 57

### N-(4′-cyano(1,1′-biphenyl)-3-yl)-8,8,8-trifluoro-7-oxooctanamide

The desired product was prepared by substituting methyl 8,8,8-trifluoro-7-oxooctanoate and 3′-amino(1,1′-biphenyl)-4-carbonitrile for Example 1A and aniline, respectively, in Example 1.

MS (ESI(−)) m/e 387 (M−H)⁻;

¹H NMR (300 MHz, DMSO-d₆) δ 10.04 (s, 1H), 8.03 (s, 1H), 7.93 (d, 2H), 7.80 (d, 2H), 7.63 (d, 1H), 7.48-7.35 (m, 2H), 2.87 (t, 2H), 2.33 (t, 2H), 1.69-1.52 (m, 4H), 1.43-1.23 (m, 2H);

Anal. Calcd for C₂₁H₁₉N₂O₂F₃.0.25H₂O: C, 64.20; H, 5.0; N, 7.13. Found: C, 64.26; H, 5.08; N, 7.13.

### Example 58

### N-(3-cyanophenyl)-8,8,8-trifluoro-7-oxooctanamide

The desired product was prepared by substituting methyl 8,8,8-trifluoro-7-oxooctanoate and 3-cyanoaniline for Example 1A and aniline, respectively, in Example 1.

MS (ESI(−)) m/e 311 (M−H)⁻;

¹H NMR (300 MHz, DMSO-d₆) δ 10.22 (s, 1H), 8.10 (s, 1H), 7.78 (dd, 1H), 7.56-7.43 (m, 2H), 2.88 (t, 2H), 2.33 (t, 2H), 1.68-1.53 (m, 2H), 1.51-1.20 (m, 4H);

Anal. Calcd for C₁₅H₅N₂O₂F₃.0.65H₂O: C, 55.61; H, 5.07; N, 8.65. Found: C, 55.67; H, 5.0; N, 8.51.

### Example 59

### 8,8,8-trifluoro-7-oxo-N-(4-phenoxyphenyl)octanamide

The desired product was prepared by substituting methyl 8,8,8-trifluoro-7-oxooctanoate and 4-phenoxyaniline for Example 1A and aniline, respectively, in Example 1.

MS (ESI(−)) m/e 378 (M−H)⁻;

¹H NMR (300 MHz, DMSO-d₆) δ 9.88 (s, 1H), 7.60 (d, 2H), 7.36 (t, 2H), 7.08 (t, 1H), 6.97 (t, 4H), 2.88 (t, 2H), 2.28 (t, 2H), 1.67-1.52 (m, 4H), 1.41-1.28 (m, 2H);

Anal. Calcd for C₂₀H₂₀NO₃F₃: C, 63.32; H, 5.31; N, 3.69. Found: C, 63.14; H, 5.22; N, 3.53.

### Example 60

### N-(4-aminobenzyl)-9,9,9-trifluoro-8-oxononanamide

The desired product was prepared by substituting 4-(aminomethyl)aniline for 4-aminopyridine in Example 6.

MS (ESI(−)) m/e 329 (M−H)⁻;

¹H NMR (300 MHz, DMSO-d₆) δ 8.06 (t, 1H), 7.22 (dt, 1H), 7.12 (d, 1H), 7.00 (d, 1H), 6.88 (t, 1H), 4.20 (d, 2H), 2.14 (t, 2H), 1.60 (m, 2H), 1.52 (m, 2H), 1.42 (m, 2H), 1.22 (m, 4H).

### Example 61

### 9,9,9-trifluoro-N-(3-methylphenyl)-8-oxononanamide

The desired product was prepared by substituting 3-methylaniline for 4-aminopyridine in Example 6.

MS (ESI(−)) m/e 314 (M−H)⁻;

¹H NMR (300 MHz, DMSO-d₆) δ 9.80 (s, 1H), 7.44 (s, 1H), 7.36 (d, 1H), 7.15 (t, 1H), 6.85 (d, 1H), 2.28 (t, 2H), 2.26 (s, 3H), 1.6 (m, 4H), 1.44 (m, 2H), 1.28 (m, 4H).

### Example 62

### 9,9,9-trifluoro-N-(4-methylphenyl)-8-oxononanamide

The desired product is prepared by substituting 4-methylaniline for 4-aminopyridine in Example 6.

MS (ESI(−)) m/e 314 (M−H)⁻;

¹H NMR (300 MHz, DMSO-d₆) δ 9.74 (s, 1H), 7.42 (d, 2H), 7.04 (d, 2H), 2.28 (t, 2H), 2.26 (s, 3H), 1.62 (m, 4H), 1.22 (m, 6H).

### Example 63

### N-(4-aminophenyl)-9,9,9-trifluoro-8-oxononanamide

The desired product was prepared by substituting 1,4-benzenediamine for 4-aminopyridine in Example 6.

MS (ESI(−)) m/e 315 (M−H)⁻;

¹H NMR (300 MHz, DMSO-d₆) δ 9.80 (s, 1H), 7.45 (d, 2H), 7.0 (d, 2H), 6.60 (br s, 2H), 2.24 (t, 2H), 1.46 (m, 4H), 1.40 (m, 2H), 1.22 (m, 4H).

### Example 64

### 9,9,9-trifluoro-N-(4-fluorobenzyl)-8-oxononanamide

The desired product was prepared by substituting (4-fluorophenyl)methanamine for 4-aminopyridine in Example 6.

MS (ESI(−)) m/e 332 (M−H)⁻;

¹H NMR (300 MHz, DMSO-d₆) δ 9.02 (t, 0.54H), 8.24 (t, 0.46H), 8.00 (m, 1H), 7.35 (m, 1H), 7.30 (m, 1H), 7.24 (m, 1H), 4.42 (d, 1.08H), 4.21 (d, 0.96H), 2.10 (dt, 2H), 1.60 (m, 2H), 1.52 (m, 2H), 1.42 (m, 2H), 1.24 (m, 4H).

### Example 65

### 9,9,9-trifluoro-N-(3-methoxybenzyl)-8-oxononanamide

The desired product was prepared by substituting (3-methoxyphenyl)methanamine for 4-aminopyridine in Example 6.

MS (ESI(−)) m/e 344 (M−H)⁻;

¹H NMR (300 MHz, DMSO-d₆) δ 9.00 (t, 1H), 7.22 (t, 1H), 7.05 (dd, 1H), 6.90 (d, 1H), 6.80 (d, 1H), 4.40 (d, 2H), 3.82 (s, 3H), 2.24 (t, 2H), 1.60 (m, 2H), 1.52 (m, 2H), 1.42 (m, 2H), 1.24 (m, 4H).

### Example 66

### 9,9,9-trifluoro-N-(4-methoxybenzyl)-8-oxononanamide

The desired product was prepared by substituting (4-methoxyphenyl)methanamine for 4-aminopyridine in Example 6.

MS (ESI(−)) m/e 344 (M−H)⁻;

¹H NMR (300 MHz, DMSO-d₆) δ 8.18 (t, 1H), 7.40 (d, 1H), 7.10 (d, 1H), 7.00 (d, 1H), 6.80 (d, 1H), 4.20 (d, 2H), 3.70 (s, 3H), 2.10 (t, 2H), 1.60 (m, 2H), 1.50 (m, 2H), 1.40 (m, 2H), 1.24 (m, 4H).

### Example 67

### 9,9,9-trifluoro-N-(3-fluorobenzyl)-8-oxononanamide

The desired product was prepared by substituting (3-fluorophenyl)methanamine for 4-aminopyridine in Example 6.

MS (ESI(−)) m/e 332 (M−H)⁻;

¹H NMR (300 MHz, DMSO-d₆) δ 9.20 (t, 0.5H), 8.30 (t, 0.5H), 7.78 (dd, 0.5H), 7.65 (dd, 0.5H), 7.54 (m, 1H), 7.36 (m, 1H), 7.15 (m, 0.5H), 7.05 (m, 0.5H), 4.45 (d, 1H), 4.22 (d, 1H), 2.30 (t, 1H), 2.14 (t, 1H), 1.60 (m, 2H), 1.52 (m, 2H), 1.42 (m, 2H), 1.24 (m, 4H).

### Example 68

### 9,9,9-trifluoro-N-(3-chlorobenzyl)-8-oxononanamide

The desired product was prepared by substituting (3-chlorophenyl)methanamine for 4-aminopyridine in Example 6.

MS (ESI(−)) m/e 348 (M−H)⁻;

¹H NMR (300 MHz, DMSO-d₆) δ 8.30 (t, 1H), 7.34 (m, 1H), 7.28 (m, 2H), 7.20 (d, 1H), 4.20 (d, 2H), 2.16 (t, 2H), 1.60 (m, 2H), 1.50 (m, 2H), 1.40 (m, 2H), 1.22 (m, 4H).

### Example 69

### N-(4-bromobenzyl)-9,9,9-trifluoro-8-oxononanamide

The desired product was prepared by substituting (4-bromophenyl)methanamine for 4-aminopyridine in Example 6.

MS (ESI(−)) m/e 392 (M−H)⁻;

¹H NMR (300 MHz, DMSO-d₆) δ 8.30 (t, 1H), 7.50 (d, 2H), 7.20 (d, 2H), 4.20 (d, 2H), 2.12 (m, 2H), 1.60 (m, 2H), 1.50 (m, 2H), 1.40 (m, 2H), 1.22 (m, 4H).

### Example 70

### N-(3-(dimethylamino)phenyl)-9,9,9-trifluoro-8-oxononanamide

The desired product was prepared by substituting N,N-dimethyl-1,3-benzenediamine for 4-aminopyridine in Example 6.

MS (ESI(−)) m/e 343 (M−H)⁻;

¹H NMR (300 MHz, DMSO-d₆) δ 9.60 (d, 1H), 7.05 (m, 2H), 6.90 (m, 1H), 6.41 (d, 1H), 2.50 (s, 6H), 2.20 (m, 2H), 1.60 (m, 2H), 1.50 (m, 2H), 1.40 (m, 2H), 1.22 (m, 4H).

### Example 71

### 9,9,9-trifluoro-8-oxo-N-(3-(trifluoromethoxy)benzyl)nonanamide

The desired product was prepared by substituting (3-(trifluoromethoxy)phenyl)-methanamine for 4-aminopyridine in Example 6.

MS (ESI(−)) m/e 398 (M−H)⁻;

¹H NMR (300 MHz, DMSO-d₆) δ 8.38 (t, 1H), 7.40 (t, 1H), 7.25 (d, 1H), 7.20 (m, 2H), 4.25 (d, 2H), 2.10 (t, 2H), 1.60 (m, 1H), 1.48 (m, 4H), 1.40 (m, 1H), 1.22 (m, 4H).

### Example 72

### 9,9,9-trifluoro-8-oxo-N-(3-(trifluoromethyl)benzyl)nonanamide

The desired product was prepared by substituting (3-(trifluoromethyl)phenyl)-methanamine for 4-aminopyridine in Example 6.

MS (ESI(−)) m/e 382 (M−H)⁻;

¹H NMR (300 MHz, DMSO-d₆) δ 8.40 (t, 1H), 7.60 (m, 4H), 4.36 (d, 2H), 2.14 (m, 2H), 1.60 (m, 4H), 1.40 (m, 1H), 1.22 (m, 4H).

### Example 73

### 9,9,9-trifluoro-8-oxo-N-(3-(trifluoromethoxy)phenyl)nonanamide

The desired product was prepared by substituting 3-(trifluoromethoxy)aniline for 4-aminopyridine in Example 6.

MS (ESI(−)) m/e 384 (M−H)⁻;

¹H NMR (300 MHz, DMSO-d₆) δ 10.10 (s, 1H), 7.52 (d, 1H), 7.40 (m, 2H), 7.0 (d, 1H), 2.30 (m, 2H), 1.60 (m, 5H), 1.40 (m, 1H), 1.20 (m, 4H).

### Example 74

### N-(3,5-dimethoxybenzyl)-9,9,9-trifluoro-8-oxononanamide

The desired product was prepared by substituting (3,5-dimethoxyphenyl)-methanamine for 4-aminopyridine in Example 6.

MS (ESI(−)) m/e 374 (M−H)⁻;

¹H NMR (300 MHz, DMSO-d₆) δ 8.20 (s, 1H), 6.56 (s, 1H), 6.40 (s, 2H), 4.20 (d, 2H), 3.60 (s, 6H), 2.12 (t, 2H), 1.60 (m, 1H), 1.54 (m, 4H), 1.40 (m, 1H), 1.22 (m, 4H).

### Example 75

### N-(2,4-dimethylphenyl)-9,9,9-trifluoro-8-oxononanamide

The desired product was prepared by substituting 2,4-dimethylaniline for 4-aminopyridine in Example 6.

MS (ESI(−)) m/e 328 (M−H⁻;

¹H NMR (300 MHz, DMSO-d₆) δ 9.10 (s, 1H), 7.20 (d, 1H), 7.00 (s, 1H), 6.95 (d, 1H), 2.24 (t, 2H), 2.20 (s, 3H), 2.14 (s, 3H), 1.60-1.40 (m, 6H), 1.28-1.20 (m, 4H).

### Example 76

### N-(3,4-dimethylphenyl)-9,9,9-trifluoro-8-oxononanamide

The desired product was prepared by substituting 3,4-dimethylaniline for 4-aminopyridine in Example 6.

MS (ESI(−)) m/e 328 (M−H);

¹H NMR (300 MHz, DMSO-d₆) δ 9.60 (s, 1H), 7.40 (s, 1H), 7.34 (d, 1H), 7.0 (d, 1H), 2.24 (t, 2H), 2.18 (s, 3H), 2.12 (s, 3H), 1.60 (m, 5H), 1.40 (m, 1H), 1.28 (m, 4H).

### Example 77

### N-(3,5-dimethylphenyl)-9,9,9-trifluoro-8-oxononanamide

The desired product was prepared by substituting 3,5-dimethylaniline for 4-aminopyridine in Example 6.

MS (ESI(−)) m/e 328 (M−H)⁻;

¹H NMR (300 MHz, DMSO-d₆) δ 9.60 (s, 1H), 7.20 (s, 2H), 6.64 (s, 1H), 2.24 (t, 2H), 2.20 (s, 6H), 1.60 (m, 5H), 1.40 (m, 1H), 1.26 (m, 4H).

### Example 78

### N-(2,4-dimethoxyphenyl)-9,9,9-trifluoro-8-oxononanamide

The desired product was prepared by substituting 2,4-dimethoxyaniline for 4-aminopyridine in Example 6.

MS (ESI(−)) m/e 360 (M−H)⁻;

¹H NMR (300 MHz, DMSO-d₆) δ 8.82 (s, 1H), 7.60 (d, 1H), 6.60 (s, 1H), 6.42 (d, 1H), 3.66 (s, 3H), 3.62 (s, 3H), 2.30 (t, 2H), 1.60 (m, 5H), 1.40 (m, 1H), 1.26 (m, 4H).

### Example 79

### N-(2,5-dimethoxybenzyl)-9,9,9-trifluoro-8-oxononanamide

The desired product was prepared by substituting (2,5-dimethoxyphenyl)methanamine for 4-aminopyridine in Example 6.

MS (ESI(−)) m/e 374 (M−H)⁻;

¹H NMR (300 MHz, DMSO-d₆) δ 8.12 (t, 1H), 6.90 (d, 1H), 6.68 (d, 1H), 6.62 (s, 1H), 4.2 (d, 2H), 3.72 (s, 3H), 3.64 (s, 3H), 2.10 (t, 2H), 1.60-1.40 (m, 6H), 1.20 (m, 4H).

### Example 80

### N-(3,5-dimethoxyphenyl)-9,9,9-trifluoro-8-oxononanamide

The desired product was prepared by substituting 3,5-dimethoxyaniline for 4-aminopyridine in Example 6.

MS (ESI(−)) m/e 360 (M−H)⁻;

¹H NMR (300 MHz, DMSO-d₆) δ 9.80 (s, 1H), 6.80 (s, 2H), 6.20 (s, 1H), 3.70 (s, 6H), 2.24 (t, 2H), 1.60 (m, 4H), 1.40 (m, 2H), 1.22 (m, 4H).

### Example 81

### N-(1,3-benzodioxol-5-ylmethyl)-9,9,9-trifluoro-8-oxononanamide

The desired product was prepared by substituting 1,3-benzodioxol-5-ylmethanamine for 4-aminopyridine in Example 6.

MS (ESI(−)) m/e 358 (M−H)⁻;

¹H NMR (300 MHz, DMSO-d₆) δ 8.80 (t, 1H), 6.86 (d, 1H), 6.82 (s, 1H), 6.76 (d, 1H), 6.0 (s, 2H), 4.20 (d, 2H), 2.10 (t, 2H), 1.80-1.40 (m, 6H), 1.20 (m, 4H).

(352492) Example 82

### 9,9,9-trifluoro-8-oxo-N-(3,4,5-trimethoxyphenyl)nonanamide

The desired product was prepared by substituting 3,4,5-trimethoxyaniline for 4-aminopyridine in Example 6.

MS (ESI(−)) m/e 390 (M−H)⁻;

¹H NMR (300 MHz, DMSO-d₆) δ 9.70 (s, 1H), 7.00 (s, 2H), 3.80 (s, 6H), 3.60 (s, 3H), 2.22 (t, 2H), 1.60 (m, 4H), 1.20 (m, 2H), 1.20 (m, 4H).

### Example 83

### N-(3,4-dichlorobenzyl)-9,9,9-trifluoro-8-oxononanamide

The desired product was prepared by substituting (3,4-dichlorophenyl)-methanamine for 4-aminopyridine in Example 6.

MS (ESI(−)) m/e 382 (M−H)⁻;

¹H NMR (300 MHz, DMSO-d₆) δ 8.40 (t, 1H), 7.60 (d, 1H), 7.42 (s, 1H), 7.20 (d, 1H), 4.22 (d, 2H), 2.12 (t, 2H), 1.62-1.40 (m, 6H), 1.20 (m, 4H).

### Example 84

### 8-((1 ,1′-biphenyl)-4-yloxy)-1,1,1-trifluoro-3-octyn-2-one

### Example 84A

### 4-(5-hexynyloxy)-1,1′-biphenyl

A solution of (1,1′-biphenyl)-4-ol (1.70 g, 10.0 mmol), 5-hexyn-1-ol (0.98 g, 10.0 mmol), and triphenylphosphine (3.41 g, 13.0 mmol) in THF (20 mL) at 0° C. was treated dropwise with diethylazodicarboxylate (2.27 g, 13.0 mmol), warmed to room temperature, stirred for 18 hours, and concentrated. The concentrate was purified by flash column chromatography on silica gel with 50:1 hexanes/ethyl acetate to provide 1.68 g (67%) of the desired product.

### Example 84B

### 8-((1,1′-biphenyl)-4-yloxy)-1,1,1-trifluoro-3-octyn-2-one

A solution of Example 84A (1.51 g, 6.0 mmol) in THF (35 mL) at −78° C. was treated dropwise with 2.5 M n-butyllithium in hexanes (2.6 mL, 6.5 mmol) and ethyl trifluoroacetate (0.98 g, 6.9 mmol), stirred for 10 minutes, treated with boron trifluoride diethyl etherate (1.50 g, 10 mmol), stirred for 4 hours, warmed to room temperature, and stirred for 18 hours. The mixture was quenched with saturated NH₄Cl (20 mL), and extracted with diethyl ether. The combined extracts were washed with brine, dried (Na₂SO₄), filtered, and concentrated. The concentrate was purified by flash column chromatography on silica gel with 95:5 to 92:8 hexanes/ethyl acetate to provide 959 mg (46%) of the desired product.

MS (ESI(−)) m/e 345 (M−H)⁻;

¹H NMR (300 MHz, DMSO-d₆) δ 7.62-7.57 (m, 4H), 7.45-7.40 (m, 2H), 7.33-7.27 (m, 1H), 7.02 (d, 2H), 4.06 (t, 2H), 2.78 (t, 2H), 1.91-1.71 (m, 4H);

Anal. Calcd for C₂₀H₁₇F₃O₂: C, 69.36; H, 4.95. Found: C, 69.45; H, 5.08.

### Example 85

### N-(4-(dimethylamino)benzyl)-9,9,9-trifluoro-8-oxononanarmide

The desired product was prepared by substituting 4-(aminomethyl)-N,N-dimethylaniline for 4-aminopyridine in Example 6.

¹H NMR (300 MHz, DMSO-d₆) δ 8.06 (t, 1H), 7.12 (d, 2H), 6.88 (d, 2H), 4.20 (d, 2H), 2.80 (s, 6H), 2.14 (t, 2H), 1.60 (m, 2H), 1.52 (m, 2H), 1.42 (m, 2H), 1.22 (m, 4H);

MS (ESI(−)) m/e 357 (M−H)⁻.

### Example 86

### (3E)-8-((1,1′-biphenyl)-4-yloxy)-1,1,1-trifluoro-3-octen-2-one

### Example 86A

### (3E)-8-((1,1′-biphenyl)-4-yloxy)-1,1,1-trifluoro-3-octen-2-ol

A suspension of LAH (201 mg, 5.3 mmol) in THF (10 mL) at 0° C. was treated dropwise with a solution of Example 84B (822 mg, 2.40 mmol) in THF (2.5 mL), stirred for 30 minutes, warmed to room temperature, heated to reflux for 8 hours, cooled to 0° C., and treated sequentially with water (0.1 mL), 1M NaOH (0.1 mL), and water (0.5 mL). The suspension was filtered and the filtrate was washed sequentially with saturated NH₄Cl, water, and brine, dried (MgSO₄), filtered, and concentrated to provide 660 mg (78%) of the desired product.

MS (ESI(+)) m/e 368 (M+NH₄)⁺.

### Example 86B

### (3E)-8-((1,1′-biphenyl)-4-yloxy)-1,1,1-trifluoro-3-octen-2-one

A solution of Example 86A (201 mg, 0.57 mmol) in dichloromethane (3 mL) at room temperature was added dropwise to a suspension of Dess-Martin reagent (894 mg, 2.10 mmol) in dichloromethane (20 mL), stirred for 3 hours, diluted with IM NaOH (20 mL) and diethyl ether (20 mL), and stirred for 30 minutes. The aqueous phase was extracted with diethyl ether and the combined organic phases were washed with brine, dried (MgSO₄), filtered, and concentrated to provide 180 mg (90%) of the desired product.

MS (ESI(−)) m/e 347 (M−H)⁻;

¹H NMR (300 MHz, DMSO-d₆) δ 7.62-7.53 (m, 4H), 7.45-7.27 (m, 4H), 7.01 (d, 2H), 6.67 (dd, 1H), 4.03 (t, 2H), 2.49-2.43 (m, 2H), 1.82-1.61 (m, 4H);

Anal. Calcd for C₂₀H₁₉F₃O₂: C, 68.96; H, 5.50. Found: C, 69.11; H, 5.42.

### Example 87

### (8E)-9-(1,1′-biphenyl)-4-yl-1,1,1-trifluoro-8-nonen-2-one

### Example 87A

### ethyl (7E)-8-(1,1′-biphenyl)-4-yl-7-octenoate

A solution of (7-ethoxy-7-oxoheptyl)(triphenyl)phosphonium bromide (599 mg, 1.2 mmol) in THF (5 mL) at 0° C. was treated with potassium tert-butoxide (115 mg, 1.2 mmol) and 4-phenylbenzaldehyde (182 mg, 1.0 mmol), stirred for 1.5 hours, warmed to room temperature, and treated with ethyl acetate and water. The aqueous phase was extracted with ethyl acetate, and the combined organic phases were washed with brine, dried (MgSO₄), filtered, and concentrated. The concentrate was purified by flash column chromatography on silica gel with 5% ethyl acetate/hexanes to provide 244mg (76%) of the desired product.

MS (ESI(+)) m/e 323 (M+H)⁺.

### Example 87B

### (8E)-9-(1,1′-biphenyl)-4-yl-1,1,1-trifluoro-8-nonen-2-one

The desired product was prepared by substituting Example 87A for Example 2A in Examples 2B and 2C.

MS (ESI(−)) m/e 345 (M−H)⁻;

¹H NMR (300 MHz, DMSO-d₆) δ 7.80-7.30 (m, 9H), 6.45 (br d, 1H), 5.69 (dt, 1H), 2.87 (t, 2H), 2.35 (dq, 2H), 1.60-1.50 (m, 2H), 1.50-1.40 (m, 2H), 1.40-1.30 (m, 2H);

Anal. Calcd for C₂₁H₂₁F₃O.0.7H₂O: C, 70.61; H, 6.26. Found: C, 70.53; H, 5.76.

### Example 88

### 1-(2-(4-((1,1′-biphenyl)-4-yloxy)butyl)cyclopropyl)-2,2,2-trifluoroethanone

### Example 88A

### 1-(2-(4-((1,1′-biphenyl)-4-yloxy)butyl)cyclopropyl)-2,2,2-trifluoroethanol

Samarium (362 mg, 2.4 mmol) was dried under vacuum with heating, purged with nitrogen, treated with THF (3 mL) and a solution of Example 87A (200 mg, 0.57 mmol) in THF (4 mL), cooled to 0° C., and treated with CH₂I₂ (0.18 mL, 2.2 mmol). The reaction was warmed to room temperature, stirred for 24 hours, and partitioned between saturated K₂CO₃ and diethyl ether. The organic phase was washed with brine, dried (MgSO₄), filtered, and concentrated. The concentrate was purified by HPLC with 20% ethyl acetate/hexanes to provide 130 mg (63%) of the desired product.

MS (APCI(+)) m/e 382 (M+NH₄)⁺.

### Example 88B

### 1-(2-(4-((1,1′-biphenyl)-4-yloxy)butyl)cyclopropyl)-2,2,2-trifluoroethanone

The desired product was prepared by substituting Example 88A for Example 86A in Example 86B.

MS (ESI(+)) m/e 380 (M+NH₄)⁺;

¹H NMR (300 MHz, DMSO-d₆) δ 7.62-7.56 (m, 4H), 7.45-7.39 (m, 2H), 7.33-7.27 (m, 1H), 7.00 (d, 2H), 4.01 (t, 2H), 2.36-2.31 (m, 1H), 1.77-1.71 (m, 2H), 1.70-1.62 (m, 1H), 1.59-1.40 (m, 5H), 1.33-1.27 (m, 1H);

Anal. Calcd for C₂₁H₂₁F₃O₂.0.3H₂O: C, 68.58; H, 5.92. Found: C, 68.37; H, 5.62.

### Example 89

### 9H-fluoren-9-ylmethyl 7,7-trifluoro-6-oxoheptylcarbamate

A suspension of Fmoc-ε-aminocaproic acid (4.97 g, 14.1 mmol) in dichloromethane (25 mL) at room temperature was treated with oxalyl chloride (2.2 mL, 25.2 mmol), stirred for 1 hour, and concentrated. The concentrate was dissolved in dichloromethane (100 mL), treated with trifluoroacetic anhydride (8.92 g, 42.4 mmol), cooled to −50° C., and treated dropwise with pyridine (5.7 mL, 70 mmol) over 5 minutes. The mixture was warmed to −25° C., stirred for 1 hour, warmed to 10° C. over 1 hour, cooled to −30° C., quenched with water (10 mL), and partitioned between dichloromethane and water. The organic phase was washed with water, dried (Na₂SO₄), filtered, and concentrated. The concentrate was purified by flash column chromatography on silica gel with 30% ethyl acetate/hexanes to 50% ethyl acetate/hexanes to 80% ethyl acetate/hexanes to provide 900 mg (16%) of the desired product. mp: 83-88° C.;

MS (ESI(+)) m/e 406 (M+H)⁺;

¹H NMR (300 MHz, CDCl₃) δ 1.39-1.27 (m, 2H), 1.56-1.43 (m, 2H), 1.63-1.58 (m, 2H), 2.68 (t, 2H), 3.09-2.92 (m, ˜0.5H), 3.17 (q, 2H), 4.20 (t, 1H), 4.40 (d, 2H), 4.54-4.43 (m, ˜0.5H), 4.89-4.76 (m, 1H), 7.28 (td, 2H), 7.37 (t, 2H), 7.57 (d, 2H), 7.73 (d, 2H);

Anal. Calcd for C₂₂H₂₂F₃NO₃: C, 65.18; H, 5.47; N, 3.45; F, 14.06. Found: C,64.94; H, 5.53; N, 3.46; F, 13.75.

### Example 90

### 4-((1,1′-biphenyl)-4-yloxy)-N-(3,3 ,3-trifluoro-2-oxopropyl)butanamide

### Example 90A

### methyl 4-((1,1′-biphenyl)-4-yloxy)butanoate

A mixture of (1,1′-biphenyl)-4-ol (2.0 g, 11.75 mmol), methyl 4-bromobutyrate (1.55 mL, 12.0 mmol) and Cs₂CO₃ (4.21 g, 12.9 mmol) in DMF (40 mL) at room temperature was stirred for 18 hours, diluted with water (500 mL), and filtered to provide 2.98 g (94%) of the desired product.

MS (ESI(+)) m/e 271 (M+H)⁺.

### Example 90B

### lithium 4-((1,1′-biphenyl)-4-yloxy)butanoate

A mixture of Example 90A (2.0 g, 8.0 mmol), 2M LiOH in water (24 mL, 48 mmol) and THF (18 mL) at room temperature was stirred for 18 hours, partially concentrated, and filtered. The solid was washed with water and dried in a vacuum oven to provide 1.94 g (93%) of the desired product.

MS (ESI(−)) m/e 255 (M−Li)⁻.

### Example 90C

### 4-((1,1′-biphenyl)-4-yloxy)-N-(3,3,3-trifluoro-2-hydroxypropyl)butanamide

A mixture of Example 90B (206 mg, 0.8 mmol), 2-hydroxy-2-(trifluoromethyl)ethylamine (102 mg, 0.79 mmol, prepared as described in J.Org.Chem. 1995, 60, 41), EDCI (162 mg, 0.85 mmol), HOBt (115 mg, 0.85 mmol) and NMM (0.17 mL, 1.5 mmol) in DMF (2 mL) at room temperature was stirred for 18 hours, and partitioned between ethyl acetate and water. The organic phase was washed with brine, dried (Na₂SO₄), filtered, and concentrated. The concentrate was purified by flash column chromatography on silica gel with 20% ethyl acetate/hexanes to 30% ethyl acetate/hexanes to 40% ethyl acetate/hexanes to provide 0.16 g (55%) of the desired product.

MS (ESI(−)) m/e 366 (M−H)⁻.

### Example 90D

### 4-((1,1′-biphenyl)-4-yloxy)-N-(3,3,3-trifluoro-2-oxopropyl)butanamide

The desired product was prepared by substituting Example 90C for Example 86A in Example 86B.

MS (ESI(+)) m/e 366 (M+H)⁺;

¹H NMR (300 MHz, DMSO-d₆) δ 8.08-8.04 (m, 1H), 7.62-7.57 (m, 4H), 7.45-7.40 (m, 2H), 7.32-7.27 (m, 1H), 7.00 (d, 2H), 4.01 (t, 2H), 3.40 (d, 2H), 2.34 (t, 2H), 1.99-1.93 (m, 2H);

Anal. Calcd for C₁₉H₁₈F₃NO₃.0.8H₂O: C, 60.09; H, 5.20; N, 3.69. Found: C, 59.74; H, 5.35; N, 3.51.

### Example 91

### methyl 8-((1,1′-biphenyl)-4-yloxy)-2-oxooctanoate

### Example 91A

### 6-((4′-phenyl)phenoxy))-hexan-1-ol

A solution of (1,1′-biphenyl)-4-ol (2.3 g, 13.5 mmol) in DMF (15 mL) at room temperature was treated with Cs₂CO₃ (8.8 g, 27 mmol), stirred for 20 minutes, treated with a solution of 6-(t-butyldimethylsilyloxy)-hexyl bromide (4.78 g, 16.2 mmol) in DMF (5 mL), stirred for 48 hours, and partitioned between water and diethyl ether. The aqueous phase was extracted with diethyl ether, and the combined organic phases were washed with brine, dried (MgSO₄), filtered, and concentrated. The concentrate was dissolved in THF (60 mL), treated with 1M TBAF in TBF (27 mL, 27 mmol), stirred for 18 hours, poured into water, and extracted with ethyl acetate. The combined extracts were washed with brine, dried (MgSO₄), filtered, and concentrated. The concentrate was triturated with hexanes and filtered to provide the desired product.

### Example 91B

### 6-((1,1′-biphenyl)-4-yloxy)hexanal

A solution of oxalyl chloride (1.24 mL, 14.22 mmol) in dichloromethane (10 mL) at −60° C. was treated dropwise with a solution of DMSO (1.85 mL, 26.1 mmol) in dichloromethane (2 mL), stirred for 10 minutes, treated with a solution of Example 91A (3.2 g, 11.85 mmol) in dichloromethane (10 mL), stirred for 15 minutes, treated with triethylamine (8.19 mL, 59.2 mmol), stirred for 5 minutes, warmed to room temperature, and partitioned between water and dichloromethane. The organic extract was washed with brine, dried (MgSO₄), filtered, and concentrated. The concentrate was purified by flash column chromatography on silica gel with 10% ethyl acetate/hexanes to provide the desired product.

### Example 91C

### methyl 8-((1,1′-biphenyl)-4-yloxy)-2-oxooctanoate

A suspension of lithium chloride (24 mg, 0.58 mmol) in acetonitrile (2 mL) at room temperature was treated with a solution of methyl (dimethoxyphosphoryl)-(tetrahydro-2H-pyran-2-yloxy)acetate (150 mg, 0.53 mmol, prepared according to the procedure described in Tet. Lett. 1981, 22, 663-666) in acetonitrile (1.5 mL), treated with DBU (0.07 mL, 0.47 mmol), stirred for 10 minutes, cooled to 0° C., treated with a solution of Example 91B (118 mg, 0.44 mmol) in acetonitrile (2 mL), stirred for 1.5 hours, and warmed to room temperature. The reaction was partitioned between water and diethyl ether and the organic extract was dried (MgSO₄), filtered, and concentrated. The concentrate was dissolved in methanol (10 mL), treated with pTsOH.H₂O (15 mg), stirred for 45 minutes, and concentrated. The concentrate was dissolved in dichloromethane, washed with saturated NaHCO₃, dried (Na₂SO₄), filtered, and concentrated. The concentrate was purified by flash column chromatography on silica gel with 10% ethyl acetate/hexanes to provide 63 mg (42%) of the desired product.

MS (ESI(+)) m/e 358 (M+NH₄)⁺;

¹H NMR (300 MHz, DMSO-d₆) δ 7.62-7.56 (m, 4H), 7.45-7.40 (m, 2H), 7.33-7.28 (m, 1H), 7.01 (d, 2H), 4.00 (t, 2H), 3.32 (s, 3H), 2.83 (t, 2H), 1.77-1.69 (m, 2H), 1.58-1.49 (m, 2H), 1.48-1.32 (m, 4H);

Anal. Calcd. for C₂₁H₂₄O₄: C, 74.09; H, 7.11. Found: C, 74.07; H, 6.93.

### Example 92

### 7-((1,1′-biphenyl)-3-yloxy)-1-(1,3-oxazol-2-yl)-1-heptanone

### Example 92A

### ethyl 7-((1,1′-biphenyl)-3-yloxy)heptanoate

The desired product was prepared by substituting (1,1′-biphenyl)-3-ol for (1,1′-biphenyl)-4-ol in Example 2A.

### Example 92B

### 7-((1,1′-biphenyl)-3-yloxy)heptanoic acid

The desired product was prepared by substituting Example 92A for Example 1A in Example 1B.

### Example 92C

### 7-((1,1′-biphenyl)-3-yloxy)heptanoyl chloride

The desired product was prepared by substituting Example 92B for Example 102B in Example 102C.

### Example 92D

### 7-((1,1′-biphenyl)-3-yloxy)-1-(1 ,3-oxazol-2-yl)-1-heptanone

A solution of oxazole (39 mg, 0.56 mmol) in THF (6 mL) at −78° C. was treated dropwise with 2.5M n-butyllithium in hexanes (0.34 mL, 0.85 rnmol), stirred for 20 minutes, treated with 0.5M ZnCl₂ in THF (2.26 mL, 1.13 mmol), warmed to ⁰° C., and stirred for 45 minutes. The mixture was treated with Cul (107 mg, 0.56 mmol), stirred for 10 minutes, treated dropwise with a solution of Example 92C (1.13 mmol) in THF (4 mL), and stirred for 1 hour. The mixture was treated with ethyl acetate (30 mL), washed sequentially with 15% NH₄OH (20 mL), water (20 mL), and saturated NH₄Cl (10 ML), dried (MgSO₄), filtered, and concentrated. The concentrate was purified by flash column chromatography on silica gel with 20% ethyl acetate/hexanes to provide the desired product.

MS (ESI(+)) m/e 350 (M+H)⁺;

¹H NMR (CDCl₃) δ 7.80 (s, 1H), 7.60 (m, 1H), 7.58 (m, 1H), 7.42 (m, 2H), 7.36 (m, 1H), 7.34 (m, 1H), 7.32 (s, 1H), 7.16 (m, 1H), 7.10(t, 1H), 6.88 (dd, 1H), 4.00 (t,2H), 3.10(t, 2H), 1.85-1.75 (m, 4H), 1.55-1.45 (m, 4H).

### Example 93

### 8-((11′-biphenyl)-4-yloxy)-2-oxooctanoic acid

The desired product was prepared by substituting Example 91C for Example 1A in Example 1B.

MS (ESI(+)) m/e 344 (M+NH4)⁺;

¹H NMR (300 MHz, DMSO-d₆) δ 7.62-7.56 (m, 4H), 7.45-7.40 (m, 2H), 7.32-7.27 (m, 1H), 7.03-7.00 (m, 2H), 4.00 (t, 2H), 2.66 (t, 2H), 1.77-1.67 (m, 2H), 1.56-1.24 (m, 6H);

Anal. Calcd. for C₂oH₂₂O₄.H₂O: C, 69.75; H, 7.02. Found: C, 69.76; H, 6.70.

### Example 94

### ethyl 7-((1,1′-biphenyl)-4-yloxy)-2-oxoheptanoate

### Example 94A

### ethyl 7-((1,1′-biphenyl)-4-yloxy)-2-hydroxyheptanoate

A solution of 0.5M KHMDS in THF (9.2 mL, 4.6 mmol) in THF (100 mL) at −78° C. was treated with a solution of Example 2A (1.0 g, 3.0 mmol) in THF (50 mL), stirred for 15 minutes, treated with 2-benzenesulfonyl-3-phenyl-oxaziridine (1.2 g, 4.6 mmol, prepared according to the procedure described in J. Org. Chem. 1982, 47, 1774-1775) stirred for 30 minutes, quenched with saturated NH₄Cl, and extracted with ethyl acetate. The combined extracts were dried (MgSO₄), filtered, and concentrated. The concentrate was purified by flash column chromatography on silica gel with 4:1 ethyl acetate/dichloromethane to provide 0.51 g (50%) of the desired product.

### Example 94B

### ethyl 7-((1,1′-biphenyl)-4-yloxy)-2-oxoheptanoate

A solution of Example 94A (155 mg, 0.45 mmol) in dichloromethane (3 mL) at 0° C. was treated sequentially with 4A molecular sieves and PDC (256 mg, 0.68 mmol), warmed to room temperature, stirred for 72 hours, diluted with ethyl acetate, filtered through diatomaceous earth (Celite®), and concentrated. The concentrate was purified by flash column chromatography on silica gel with 8:1 ethyl acetate/hexanes to provide the desired product.

¹H NMR (300 MHz, CDCl₃) δ 7.56-7.49 (m, 4H), 7.44-7.38 (m, 2H), 7.32-7.27 (m, 1H), 6.98-6.93 (m, 2H), 4.32 (q, 2H), 4.00 (t, 2H), 2.89 (t, 2H), 1.88-1.69 (m, 4H), 1.60-1.55 (m, 2H), 1.37 (t, 3H);

MS (DCI/NH₃) m/e 358 (M+NH₄)⁺;

Anal. Calcd. for C₂₁H₂₄O₄: C, 74.09; H, 7.11. Found: C, 74.10; H, 7.03.

### Example 95

### ethyl 7-((3-bromo(1,1′-biphenyl)-4-yl)oxy)-2-oxoheptanoate

### Example 95A

### 4-((5-bromopentyl)oxy)-1,1′-biphenyl

A solution of 1,5-dibromopentane (6.4 mL, 50 mmol) and (1,1′-biphenyl)-4-ol (2.67 g, 15.7 mmol) in DMF (50 mL) at room temperature was treated with Cs₂CO₃ (5.13 g, 15.7 mmol), stirred for 16 hours, poured into water, and extracted with ethyl acetate. The combined organics were dried (Na₂SO₄), filtered, and concentrated. The concentrate was purified by flash column chromatography on silica gel with 98:2 hexanes/ethyl acetate to provide 2.4 g (48%) of the desired product.

### Example 95B

### ethyl 2-(5-((1,1′-biphenyl)-4-yloxy)pentyl)-13-dithiane-2-carboxylate

A suspension of NaH (224 mg, 8.9 mmol) in toluene (7 mL) at 0° C. was treated sequentially with ethyl-2-dithiane carboxylate (1.03 mL, 6.53 mmol) and a solution of Example 95A (2.3 g, 7.2 mmol) in DMF (2 mL), warmed to room temperature, stirred for 16 hours, poured into water, and extracted with ethyl acetate. The combined extracts were dried (Na₂SO₄), filtered, and concentrated. The concentrate was purified by flash column chromatography on silica gel with 98:2 hexanes/ethyl acetate to provide 0.52g (19%) of the desired product.

### Example 95C

### ethyl 7-((3-bromo(1,1′-biphenyl)-4-yl)oxy)-2-oxoheptanoate

A solution of NBS (1.92g, 10.8 mmol) in a 97:3 mixture of acetone/water (19 mL) at 0° C. was treated dropwise with a solution of Example 95B (0.52g, 1.2 mmol) in a 97:3 mixture of acetone/water (3 mL), stirred for 15 minutes, and partitioned between dichloromethane and 10% Na₂SO₃. The organic phase was washed sequentially with 10% Na₂SO₃, water, saturated NaHCO₃, and brine, dried (Na₂SO₄), filtered, and concentrated. The concentrate was purified by flash column chromatography on silica gel with dichloromethane to provide 0.326 g (65%) of the desired product.

MS (ESI(+)) m/e 419 (M+H)⁺;

¹H NMR (300 MHz, DMSO-d₆) δ 7.85 (d, 1H), 7.65-7.61 (m, 3H), 7.47-7.41 (m, 2H), 7.18 (d, 2H), 4.22 (q, 2H), 4.09 (t, 2H), 2.86 (t, 2H), 1.79-1.77 (m, 2H), 1.63-1.55 (m, 2H), 1.52-1.45 (m, 2H), 1.26 (t, 3H);

Anal. Calcd. for C₂₁H₂₃BrO₄.0.5H₂O: C, 58.89; H, 5.65. Found: C, 58.73; H, 5.37.

### Example 96

### 8-(1,3-oxazol-2-yl)-8-oxo-N-phenyloctanamide

### Example 96A

### methyl 8-chloro-8-oxooctanoate

The desired product was prepared by substituting 8-methoxy-8-oxooctanoic acid for Example 102B in Example 102C.

### Example 96B

### methyl 8-(1,3-oxazol-2-yl)-8-oxooctanoate

The desired product was prepared by substituting Example 96A for Example 92C in Example 92D.

### Example 96C

### 8-(1,3-oxazol-2-yl)-8-oxooctanoic acid

The desired product was prepared by substituting Example 96B for Example 1A in Example 1B.

### Example 96D

### 8-(1,3-oxazol-2-yl)-8-oxo-N-phenyloctanamide

The desired product was prepared by substituting Example 96C for Example 1B in Example 1C.

MS (ESI(+)) m/e 301 (M+H)⁺;

¹H NMR (300 MHz, DMSO-d₆) δ 9.80 (s, 1H), 8.40 (s, 1H), 7.60 (d, 2H), 7.50 (s, 1H), 7.32 (t, 2H), 7.00 (t, 1H), 3.00 (t, 2H), 2.28 (t, 2H), 1.60 (m, 4H), 1.32 (m, 4H).

### Example 97

### N-(1,1′-biphenyl)-3-yl-8-(1,3-oxazol-2-yl)-8-oxooctanamide

The desired product was prepared by substituting Example 96C and (1,1′-biphenyl)-3-amine for Example 1B and aniline, respectively, in Example 1C.

MS (ESI(+)) m/e 377 (M+H)⁺;

¹H NMR (300 MHz, DMSO-d₆) δ 9.90 (s, 1H), 8.40 (s, 1H), 7.90 (s, 1H), 7.60 (dd, 2H), 7.50 (s, 1H), 7.48 (t, 2H), 7.30 (m, 4H), 3.00 (t, 2H), 2.28 (t, 2H), 1.60 (m, 4H), 1.32 (m, 4H).

### Example 98

### N-(4-chlorophenyl)-8-(1,3-oxazol-2-yl)-8-oxooctanamide

The desired product was prepared by substituting Example 96C and 4-chloroaniline for Example 1B and aniline, respectively, in Example 1C.

MS (ESI(+)) m/e 335 (M+H)⁺;

¹H NMR (300 MHz, DMSO-d₆) δ 10.00 (s, 1H), 8.40 (s, 1H), 7.60 (d, 2H), 7.50 (s, 1H), 7.30 (d, 2H), 3.00 (t, 2H), 2.28 (t, 2H), 1.60 (m, 4H), 1.32 (m, 4H).

### Example 99

### 8-(1,3-oxazol-2-yl)-8-oxo-N-(4-phenoxyphenyl)octanamide

The desired product was prepared by substituting Example 96C and 4-phenoxyaniline for Example 1B and aniline, respectively, in Example 1C.

MS (ESI(+)) m/e 393 (M+H)⁺;

¹H NMR (300 MHz, DMSO-d₆) δ 9.82 (s, 1H), 8.40 (s, 1H), 7.60 (d, 2H), 7.50 (s, 1H), 7.40 (m, 2H), 7.10 (t, 1H), 7.00 (m, 4H), 3.00 (t, 2H), 2.28 (t, 2H), 1.60 (m, 4H), 1.34 (m, 4H).

### Example 100

### 8-(1,3-oxazol-2-yl)-8-oxo-N-(2-pyridinyl)octanamide

The desired product was prepared by substituting Example 96C and 2-aminopyridine for Example 1B and aniline, respectively, in Example 1C.

MS (ESI(+)) m/e 302 (M+H)⁺;

¹H NMR (300 MHz, DMSO-d₆) δ 10.50 (s, 1H), 8.40 (s, 1H), 8.30 (m, 1H), 8.00 (d, 1H), 7.80 (m, 1H), 7.50 (s, 1H), 7.10 (m, 1H), 3.00 (t, 2H), 2.32 (t, 2H), 1.60 (m, 4H), 1.34 (m, 4H).

### Example 101

### 8-((1,1′-biphenyl)-4-yloxy)-N-methyl-2-oxooctanamide

### Example 101A

### mthyl 7-((1,1′-biphenyl)-4-yloxy)heptanoate

The desired product was prepared by substituting methyl 7-bromoheptanoate for ethyl 7-bromoheptanoate in Example 2A.

### Example 101B

### methyl 7-((1,1′-biphenyl)-4-yloxy)-2-hydroxyheptanoate

The desired product was prepared by substituting Example lOlA for Example 2A in Example 94A.

### Example 101C

### 8-((1,1′-biphenyl)-4-yloxy)-2-hydroxy-N-methyloctanamide

A suspension of Example 101B (20 mg, 0.06 mmol) and 2M dimethylamine in methanol (0.3 mL, 0.60 mmol) at room temperature was stirred for 48 hours and concentrated to provide 18 mg of the desired product.

MS (ESI(+)) m/e 342 (M+H)⁺.

### Example 101D

### 8-((1,1′-biphenyl)-4-yloxy)-N-methyl-2-oxooctanamide

The desired product was prepared by substituting Example lOlA for Example 86A in Example 86B.

MS (ESI(+)) m/e 340 (M+H)⁺;

¹H NMR (DMSO-d₆) δ 7.62-7.56 (m, 4H), 7.45-7.40 (m, 2H), 7.33-7.27 (m, 1H), 7.03-6.98 (m, 2H), 4.00 (t, 2H), 2.82 (t, 2H), 2.64 (d, 3H), 1.77-1.68 (m, 2H), 1.58-1.30 (m, 6H);

Anal. Calcd. for C₂₁H₂₅NO₃: C, 74.31; H, 7.42; N, 4.13. Found: C, 74.15; H, 7.60; N, 3.97.

### Example 102

### 1-(1,3-oxazol-2-yl)-7-(phenylsulfanyl)-1-heptanone

### Example 102A

### ethyl 7-(phenylsulfanyl)heptanoate

The desired product was prepared by substituting thiophenol for (1,1′-biphenyl)-4-ol in Example 2A.

### Example 102B

### 7-(phenylsulfanyl)heptanoic acid

The desired product was prepared by substituting Example 102A for Example 1A in Example 1B.

### Example 102C

### 7-(phenylsulfanyl)heptanoyl chloride

A solution of Example 102B (476 mg, 2 mmol) in dichloromethane at room temperature was treated with oxalyl chloride (0.26 mL, 3 mmol), stirred for 3 hours, and concentrated to provide the desired product.

### Example 102D

### 1-(1,3-oxazol-2-yl)-7-(phenylsulfanyl)-1-heptanone

The desired product was prepared by substituting Example 102C for Example 92C in Example 92D.

mp: 39-40° C.;

MS (ESI(+)) m/e 290 (M+H)⁺;

¹H NMR(CDCl₃): 7.82 (m,1H), 7.40-7.10 (m, 6H), 3.08 (t, 2H), 2.93 (t, 2H), 1.90-1.30 (m, 8H);

Anal. Calcd. for C₁₆H₁₉NO₂S: C, 66.40; H, 6.62, N, 4.61. Found: C, 66.06; H, 6.31; N, 4.61.

### Example 103

### 1-(1,3-oxazol-2-yl)-7-(phenylsulfonyl)-1-heptanone

A solution of Example 102D (230 mg, 0.8 mmol) in 2:1 methanol:water (20 mL) at room temperature was treated with Oxone® (1.22 g, 2 mmol) and NaHCO₃ (0.168 g, 2 mmol), stirred for 2 hours, and concentrated. The concentrate was partitioned between water and diethyl ether and the organic extract was washed with brine, dried (Na₂SO₄), filtered, and concentrated to providel80 mg (70%) of the desired product.

mp: 55-56° C.;

MS (ESI(+)) m/e 322 (M+H)⁺;

¹H NMR(DMSO-d₆): 8.39(s, 1H), 8.00-7.60 (m, 5H), 7.52 (s,1H), 3.30 (m, 2H, overlap with H₂O ), 2.98 (t, 2H), 1.70-1.20 (m, 8H);

Anal. Calcd. for C₁₆H₁₉NO₄S: C, 59.79; H, 5.96; N 4.36. Found: C, 59.51;, H, 6.13; N, 4.10.

### Example 104

### 7-(2-naphthylsulfanyl)-1-(1,3-oxazol-2-yl)-1-heptanone

### Example 104A

### ethyl 7-(2-naphthylsulfanyl)heptanoate

The desired product was prepared by substituting 2-naphthalenethiol for (1,1′-biphenyl)-4-ol in Example 2A.

### Example 104B

### 7-(2-naphthylsulfanyl)heptanoic acid

The desired product was prepared by substituting Example 104A for Example 1A in Example 1B.

### Example 104C

### 7-(2-naphthylsulfanyl)heptanoyl chloride

The desired product was prepared by substituting Example 104B for Example 102B in Example 102C.

### Example 104D

### 1-(1,3-oxazol-2-yl)-7-(phenylsulfanyl)-1-heptanone

The desired product was prepared by substituting Example 104C for Example 92C in Example 92D.

mp.65-66°;

MS (ESI(+)) m/e 340 (M+H)⁺;

¹H NMR(DMSO-d₆): 8.38 (s, 1H), 7.90-7.70 (m, 4H), 7.55-7.45 (m, 4H), 3.15-2.95 (m, 4H), 1.70-1.30 (m, 8H);

Anal. Calcd. for C₂₀H₂₁NO₂S: C, 70.77; H, 6.24; N, 4.13. Found: C, 70.78; H, 6.47, N, 3.87.

### Example 105

### 7-(2-naphthylsulfonyl)-1-(1,3-oxazol-2-yl)-1-heptanone

The desired product was prepared by substituting Example 104D for Example 102D in Example 103.

mp. 75-76° C.

MS (ESI(+)) m/e 372 (M+H)⁺;

¹H NMR(DMSO-d₆): 8.60-7.50 (m, 9H), 3.35 (m, 2H, overlap with H₂O ), 2.98 (t, 2H), 1.65-1.20 (m, 8H);

Anal. Calcd. for C₂₀H₂₁NO₄S: C, 64.67;H, 5.70;N, 3.77. Found: C, 64.39; H, 5.89; N, 3.53.

### Example 106

### N-methyl-8-(2-naphthylsulfanyl)-2-oxooctanamide

### Example 106A

### methyl 8-(2-naphthylsulfanyl)-2-oxooctanoate

The desired product was prepared by substituting 2-naphthalenethiol for 4-phenylphenol in Example 91.

### Example 106B

### N-methyl-8-(2-naphthylsulfanyl)-2-oxooctanamide

A solution of Example 106A (0.8 g, 2.4 mmol) in THF (5 mL) at room temperature was treated with 2M methylamine in THF (2.4 mL, 4.8 mmol) and triethylamine (7 mL), stirred for 4 hours, and concentrated. Recrystallization from ethyl acetate/hexanes provided 0.55g (69%) of the desired product.

mp: 94-95° C.;

MS (ESI(−)) m/e 328 (M−H)⁻;

¹H NMR (DMSO-d₆) δ 8.50 (br s, 1H), 7.90-7.30 (m, 7H), 3.07 (t, 2H), 2.79 (t, 2H), 2.64 (d, 3H), 1.70-1.20 (m, 8H);

Anal. Calcd. for C₁₉H₂₃NO₂S: C, 69.27; H, 7.04; N, 4.25. Found: C, 68.92; H, 6.93; N, 4.05.

### Example 107

### N-methyl-8-(2-naphthylsulfonyl)-2-oxooctanamide

The desired product was prepared by substituting Example 106B for Example 102D in Example 103.

mp. 108-109° C.

MS (ESI(−)) m/e 360 (M−H)⁺.

¹H NMR (DMSO-d₆) δ 8.66 (s, 1H), 8.46 (br s, 1H), 8.30-7.60 (m, 6H), 3.35 (m, 2H, overlap with H20), 2.74 (t, 2H), 2.62 (d, 3H), 1.70-1.10 (m,8H);

Anal. Calcd. for C₁₉H₂₃NO₄S: C, 63.13; H, 6.41; N 3.88. Found: C, 62.93; H, 6.26; N, 3.53.

### Example 108

### 8-((1,1′-biphenyl)-4-ylsulfanyl)-N-methyl-2-oxooctanamide

### Example 108A

### methyl 8-((1,1′-biphenyl)-4-ylsulfanyl)-2-oxooctanoate

The desired product was prepared by substituting (1,1′-biphenyl)-4-thiol for (1,1′-biphenyl)-4-ol in Example 91.

### Example 108B

### 8-((1,1′-biphenyl)-4-ylsulfanyl)-N-methyl-2-oxooctanamide

The desired product was prepared by substituting Example 108A for Example 106A in Example 106B.

mp: 131-132° C.;

MS (ESI(−)) m/e 354 (M−H)⁻;

¹H NMR (DMSO-d₆) δ 8.50 (br s, 1H), 7.70-7.30 (m, 9H), 3.00 (t, 2H), 2.79 (t, 2H), 2.62 (d, 3H), 1.70-1.20 (m, 8H);

Anal. Calcd. for C₂₁H₂₅NO₂S: C, 70.95; H, 7.09; N, 3.94. Found: C 71.05, H 7.13, N, 3.79.

### Example 109

### 8-((1,1′-biphenyl)-4-ylsulfonyl)-N-methyl-2-oxooctanamide

The desired product was prepared by substituting Example 108B for Example 102D in Example 103.

mp: 134-135° C.;

MS (ESI(−)) m/e 388 (M−H)⁻;

¹H NMR (DMSO-d₆) δ 8.50(br s, 1H), 7.80-7.40 (m, 9H), 3.40 (m, 2H, overlap with H20), 2.75 (t, 2H), 2.62(d, 3H), 1.60-1.20 (m, 8H).

### Example 110

### N-(7-(1 ,3-oxazol-2-yl)-7-oxoheptyl)-1H-indole-2-carboxamide

### Example 110A

### methyl 7-((1H-indol-2-ylcarbonyl)amino)heptanoate

The desired product was prepared by substituting methyl 7-aminoheptanoate and 1H-indole-2-carboxylic acid for Example 1B and aniline, respectively, in Example 1C.

### Example 110B

### 7-((1H-indol-2-ylcarbonyl)amino)heptanoic acid

The desired product was prepared by substituting Example 110A for Example 1A in Example 1B.

### Example 110C

### 7-((1H-indol-2-ylcarbonyl)amino)heptanoyl chloride

The desired product was prepared by substituting Example 110B for Example 102B in Example 102C.

### Example 110D

### N-(7-(1,3-oxazol-2-yl)-7-oxoheptyl)-1H-indole-2-carboxamide

The desired product was prepared by substituting Example 110C for Example 92C in Example 92D.

mp: 153-156° C.;

MS (ESI(+)) m/e 340 (M+H)⁺;

¹H NMR (DMSO-d₆) δ 1.40-1.30 (m, 4H), 1.70-1.50 (m, 4H), 3.06-3.01 (t, 2H), 3.34-3.24 (m, 2H), 7.04-6.99 (t, 1H), 7.09-7.08 (d, 1H), 7.18-7.13 (t, 1H), 7.43-7.40 (d, 1H), 7.52 (s, 1H), 7.60-7.58 (d, 1H), 8.38 (s, 1H), 8.44-8.40 (t, 1H), 11.51 (s, 1H);

Anal. Calcd. for: C₁₉H₂₁N₃O₃: C, 67.24; H, 6.24; N, 12.38. Found: C, 66.98; H, 6.13; N, 12.05.

### Example 111

### N-(6-(1,3-oxazol-2-yl)-6-oxohexyl)-1H-indole-2-carboxamide

### Example 111A

### methyl 6-((1H-indol-2-ylcarbonyl)amino)hexanoate

The desired product was prepared by substituting methyl 6-aminohexanoate and 1H-indole-2-carboxylic acid for Example 1B and aniline, respectively, in Example 1C.

### Example 111B

### 6-((1H-indol-2-ylcarbonyl)amino)hexanoic acid

The desired product was prepared by substituting Example 111A for Example 1A in Example 1B.

### Example 111C

### 6-((1H-indol-2-ylcarbonyl)arnino)hexanoyl chloride

The desired product was prepared by substituting Example 111B for Example 102B in Example 102C.

### Example 111D

### N-(6-(1,3-oxazol-2-yl)-6-oxohexyl)-1H-indole-2-carboxamide

The desired product was prepared by substituting Example 1 IC for Example 92C in Example 92D.

mp: 176-179° C.;

MS (ESI(+)) m/e 326 (M+H)⁺;

¹H NMR (DMSO-d₆) δ 1.43-1.35 (m, 2H), 1.73-1.52 (m, 4H), 3.07-3.03 (t, 2H), 7.04-6.99 (t, 1H), 7.08 (s, 1H), 7.19-7.14 (t, 1H), 7.43-7.40 (d, 1H), 7.52 (s, 1H), 7.61-7.58 (d, 1H), 8.38 (s, 1H), 8.44-8.40 (t, 1H), 11.51 (s, 1H);

Anal. Calcd. for C₁₈H₁₉N₃O₃.0.25H₂O: C, 65.54; H, 5.95; N, 12.73. Found: C, 65.63; H, 5.78; N, 12.88.

### Example 112

### 7-((1,1′-biphenyl)-4-yloxy)-1-(4,5-dihydro-1 ,3-oxazol-2-yl)-1-heptanone

### Example 112A

### 7-((4′-phenyl)phenoxy-heptan-1-ol

The desired product was prepared by substituting 7-(t-butyldimethylsilyloxy)-heptyl bromide for 6-(t-butyldimethylsilyloxy)-hexyl bromide in Example 91A.

### Example 112B

### 7-((11′-biphenyl)-4-yloxy)heptanal

The desired product was prepared by substituting Example 112A for Example 91A in Example 91B.

### Example 112C

### 8-((1,1′-biphenyl)-4-yloxy)-2-hydroxyoctanenitrile

A mixture of Example 112B (2.0g, 7.1 mmol) and KCN (4.66 g) in THF (25 mL) and water (27 mL) at room temperature was stirred for 2 days and concentrated. The resulting aqueous suspension was filtered to provide the desired product.

MS (ESI(+)) m/e 327 (M+NH4)⁺.

### Example 112D

### 7-((1,1′-biphenyl)-4-yloxy)-1-(4,5-dihydro-1,3-oxazol-2-yl)-1-heptanol

A solution of acetyl chloride (2.86 mL) in ethanol (2.8 mL) and CHCl₃ (5.7 mL) at room temperature was treated with a solution of Example 112C (0.76 g, 2.5 mmol) in CHCl₃ (8 mL), stirred overnight, and concentrated. The concentrate was suspended in dichloromethane (12 mL), treated with ethanolamine (0.3 mL) and Et₃N (0.68 mL), stirred for 24 hours, and concentrated. The concentrate was suspended in CHCl₃ (50 mL), treated with TsOH.H₂O (46 mg), heated to reflux, and stirred for 3 hours. The reaction was cooled to room temperature, diluted with ethyl acetate, washed sequentially with saturated NaHCO₃, water, and brine, dried (Na₂SO₄), filtered, and concentrated. The concentrate was purified by flash column chromatography on silica gel with 5% methanol/dichloromethane to provide 0.1 g of the desired product.

MS (ESI(+)) m/e 354 (M+H)⁺.

### Example 112E

### 7-((1,1′-biphenyl)-4-yloxy)—(4,5-dihydro-1,3-oxazol-2-yl)-1-heptanone

The desired product was prepared by substituting Example 112D for Example 86A in Example 86B.

MS (ESI(+)) m/e 352 (M+H)⁺;

¹H NMR (CDCl₃) δ 7.56-7.49 (m, 4H), 7.43-7.38 (m, 2H), 7.32-7.27 (m, 1H), 6.98-6.93 (m, 2H), 4.42 (t, 2H), 4.08 (t, 2H), 3.99 (t, 2H), 2.94 (t, 2H), 1.85-1.66 (m, 4H), 1.57-1.39 (m, 4H).

Anal. Calcd. for C₂₂H₂₅NO₃: C, 75.19; H, 7.17; N, 3.99. Found: C, 75.32; H, 6.98; N, 3.89.

### Example 113

### 9-((1,1′-biphenyl)-4-yloxy)-2,3-nonanedione

### Example 113A

### 4-((5-iodopentyl)oxy)-1,1′-biphenyl

A suspension of 5-((4′-phenyl)phenoxy))-pentan-1-ol (0.85 g, 3.3 mmol) in dichloromethane (20 mL) was treated with methanesulfonyl chloride (0.28 mL, 3.6 mmol) and Et₃N (0.7 mL, 5 mmol), stirred at 0° C. for 1 hour, and partitioned between water and dichloromethane. The organic extract was washed with water, dried (Na₂SO₄), filtered, and concentrated. The concentrate was dissolved in acetone (30 mL), treated with Nal (2.5 g, 16.6 mmol), heated to 70° C. for 18 hours, cooled to room temperature, diluted with water, and extracted three times with ethyl acetate. The combined organic extracts were washed sequentially with aqueous NaS₂O₃, water, and brine, dried (MgSO₄), filtered, and concentrated to provide 1.2 g of the desired product.

MS (ESI(+)) m/e 384 (M+NH₄)⁺.

### Example 113B

### methyl 7-((1,1′-biphenyl)-4-yloxy)-2-(2,2-dimethoxypropanoyl)heptanoate

A solution of NaH (64 mg, 1.6 mmol) in DMF (3 mL) at 0° C. was treated dropwise with methyl 4,4-dimethoxy-3-oxopentanoate (0.29 mL, 1.7 mmol), stirred for 1 hour, treated with a solution of Example 113A (0.5 g, 1.37 mmol) in DMF (4 mL), warmed to room temperature, and stirred for 36 hours. The reaction was quenched with water, and extracted 3 times with ethyl acetate. The combined organic extracts were washed with water and brine, dried (MgSO₄), filtered, and concentrated. The concentrate was purified by flash column chromatography on silica gel with 15% ethyl acetate/hexanes to provide 0.46 g (79%) of the desired product.

### Example 113C

### 9-((1,1′-biphenyl)-4-yloxy)-2,2-dimethoxy-3-nonanone

A solution of Example 113B (134 mg, 0.31 mmol) in methanol (4 mL) at room temperature was treated with 2N NaOH (0.63 mL), stirred for 3 hours, heated to 70° C., stirred for 2 hours, cooled to room temperature, diluted with water, and extracted 3 times with ethyl acetate. The combined organic extracts were washed with water and brine, dried (MgSO₄), filtered, and concentrated to provide 92 mg (80%) of the desired product. mp: 48° C.

### Example 113D

### 9-((1,1′-biphenyl)-4-yloxy)-2,3-nonanedione

A solution of Example 113C (67 mg, 0.18 mmol) in THF (2 mL) and 4N HCl (1 mL) was heated at 40° C. for 1.5h. The reaction was cooled to r.t., diluted with water, extracted 2 times with EtOAc. The combined organic extracts were washed with water, brine, dried (MgSO4), concentrated. The residue was triturated with hexane to give 43 mg (73% yield) of the title compound.

mp: 110-111 ° C.;

MS (CI(+)) m/e 342 (M+NH₄)⁺;

¹H NMR (DMSO-d₆) δ 7.60 (m, 4H), 7.43 (t, 2H), 7.30 (t, 1H), 7.01 (d, 2H), 4.00 (t, 2H), 2.70 (t, 2H), 2.23 (s, 3H), 1.72 (m, 2H), 1.51 (quint, 2H), 1.48-1.30 (m, 4H);

Anal. Calcd. for C₂₁H₂₄O₃: C, 77.75; H, 7.46. Found: C, 77.52; H, 7.19.

### (384723) Example 114

### N-(1,1′-biphenyl)-4-yl-7,8-dioxononanamide

### Example 114A

### 6-ethyl 1-methyl 2-(2,2-dimethoxypropanoyl)hexanedioate

The desired product was prepared by substituting ethyl 4-bromobutanoate for Example 113A in Example 113B.

### Example 114B

### ethyl 7,7-dimethoxy-6-oxooctanoate

The desired product was prepared by substituting Example 114A for Example 113B in Example 113C.

### Example 114C

### N-(1,1′-biphenyl)-4-yl-7,7-dimethoxy-6-oxooctanamide

The desired product was prepared by substituting Example 114B and (1,1′-biphenyl)-4-amine for Example 1B and aniline, respectively, in Example IC.

### Example 114D

### N-(1,1′-biphenyl)-4-yl-7,8-dioxononanamide

The desired product was prepared by substituting Example 114C for Example 113C in Example 113D.

¹H NMR (CDCl₃) δ 7.62-7.52 (m, 4H), 7.42 (t, 2H), 7.32 (t, 1H), 7.02 (br s, 1H), 2.77 (t, 2H), 2.39 (t, 2H), 2.33 (s, 3H), 1.77 (quint, 2H), 1.65 (quint, 2H), 1.48-1.36 (m, 2H);

MS (CI(+)) m/e 355 (M+NH₄)⁺.

### (367357) Example 115

### methyl 7-((1,1′-biphenyl)-4-yloxy)-2-oxoheptanoate

The desired product was prepared by substituting 7-(t-butyldimethylsilyloxy)-heptanyl bromide for 6-(t-butyldimethylsilyloxy)-hexyl bromide in Example 91.

MS (ESI(+)) m/e 327 (M+H)⁺;

¹NMR (DMSO-d₆) δ 7.62-7.56 (m, 4H), 7.44-7.39 (t, 2H), 7.32-7.27 (m, 1H), 7.00 (d, 2H), 4.00 (t, 2H), 3.77 (s, 3H), 2.86 (t, 2H), 1.78-1.69 (m, 2H), 1.63-1.53 (m, 2H), 1.49-1.40 (m, 2H).

### (367626) Example 116

### methyl 9-((1,1′-biphenyl)-3-ylamino)-2,9-dioxononanoate

### Example 116A

### N-(1,1′-biphenyl)-3-yl-6-(1,3-dioxolan-2-yl)hexanamide

The desired product was prepared by substituting 6-(1,3-dioxolan-2-yl)hexanoic acid and (1,1′-biphenyl)-3-amine for Example 1B and aniline, respectively, in Example

MS (ESI(+)) m/e 340 (M+H)⁺.

### Example 116B

### N-(1,1′-biphenyl)-3-yl-7-oxoheptanamide

A solution of Example 116A (2.35g, 6.9 mmol) in acetone (20 mL) and water (2 mL) was treated with TsOH.H₂O (15 mg), heated to reflux, stirred overnight, cooled to room tempearature, and concentrated. The concentrate was dissolved in ethyl acetate, washed with water, dried (Na₂SO₄), filtered, and concentrated to provide 1.82 g (89%) of the desired product.

MS (ESI(+)) m/e 296 (M+H)⁺.

### Example 116C

### methyl 9-((1,1′-biphenyl)-3-ylamino)-2,9-dioxononanoate

The desired product was prepared by substituting Example 116B for Example 91B in Example 91C.

MS (ESI(+)) m/e 368 (M+H)⁺;

¹H NMR (DMSO-d₆) δ 9.95 (s, 1H), 7.92 (br s, 1H), 7.62-7.55 (m, 311), 7.50-7.43 (m, 2H), 7.40-7.28 (m, 3H), 3.76 (s, 3H), 2.82 (t, 2H), 2.32 (t, 2H), 1.64-1.47 (m, 4H), 1.46-1.37 (m, 4H).

### Example 117

### methyl 9-anilino-2,9-dioxononanoate

### Example 117A

### 6-hvdroxy-N-phenylhexanamide

The desired product was prepared by substituting 6-hydroxyhexanoic acid for Example 1B in Example 1C.

### Example 117B

### methyl 9-anilino-2,9-dioxononanoate

The desired product was prepared by substituting Example 117A for Example 91A in Example 91.

MS (ESI(+)) m/e 292 (M+H)⁺;

¹H NMR (DMSO-d₆) δ 9.82 (s, 1H), 7.57 (d, 2H), 7.27 (t, 2H), 7.01 (t, 1H), 3.76 (s, 3H), 2.81 (t, 2H), 2.28 (t, 2H), 1.62-1.48 (m, 4H), 1.33-1.26 (m, 4H).

### Example 118

### methyl 8-((4′-cyano(1,1′-biphenyl)-4-yl)oxy)-2-oxooctanoate

The desired product was prepared by substituting 4′-hydroxy(1,1′-biphenyl)-4-carbonitrile for (1,1′-biphenyl)-4-ol in Example 91.

MS (ESI(−)) m/e 364 (M−H)⁻.

### Example 119

### 8-((4′-cyano(1,1′-biphenyl)-4-yl)oxy)-N-methyl-2-oxooctanamide

A suspension of Example 118 (261 mg, 0.7 mmol) and MeNH₂.HCl (150 mg, 2.2 mmol) in CH₃CN (5 mL) and Et₃N (10 mL) at room temperature was stirred in a sealed vessel for 18 hours, then partitioned between water and ethyl acetate. The organic extract was dried (Na₂SO₄), filtered, and concentrated. The concentrate was purified by flash column chromatography on silica gel with 30% ethyl acetate/hexanes to provide 113 mg (44%) of the desired product.

MS (ESI(−)) m/e 363 (M−H)⁻;

¹H NMR (DMSO-d₆) δ 8.53-8.48 (m, 1H), 7.90-7.83 (m, 4H), 7.70 (d, 2H), 7.05 (d, 2H), 4.04-4.00 (m, 2H), 2.82 (t, 2H), 2.64 (d, 3H), 1.78-1.68 (m, 2H), 1.58-1.47 (m, 2H), 1.47-1.30 (m, 4H).

### Example 120

### N⁹-(1,1′-biphenyl)-3-vl-N¹-methyl-2-oxononanediamide

The desired product was prepared by substituting Example 116 for Example 118 in Example 119.

MS (ESI(+)) m/e 367 (M+H)⁺;

¹H NMR (DMSO-d₆) δ 9.95 (s, 1H), 8.53-8.48 (m, 1H), 7.92-7.90 (m, 1H), 7.61-7.54 (m, 3H), 7.50-7.43 (m, 2H), 7.40-7.28 (m, 3H), 2.80 (t, 2H), 2.64 (d, 3H), 2.32 (t, 2H), 1.63-1.55 (m, 2H), 1.55-1.47 (m, 2H), 1.34-1.28 (m, 4H).

### Example 121

### (7E)-8-(1,1′-biphenyl)-4-yl-1-(1,3-oxazol-2-yl)-7-octen-1-one

### Example 121A

### (7E)-8-(1,1′-biphenyl)-4-yl-7-octenoic acid

The desired product was prepared by substituting Example 87A for Example 1A in Example 1B.

### Example 121B

### (7E)-8-(1,1′-biphenyl)-4-yl-7-octenoyl chloride

The desired product was prepared by substituting Example 121A for Example 102B in Example 102C.

### Example 121D

### (7E)-8-(1,1′-biphenyl)-4-yl-1-(1,3-oxazol-2-yl)-7-octen-1-one

The desired product was prepared by substituting Example 121C for Example 92C in Example 92D.

MS (ESI(+)) m/e 346 (M+H)⁺;

¹H NMR (DMSO-d₆) δ 8.39 (s, 1H), 7.70-7.64 (m, 4H), 7.52 (s, 1H), 7.50-7.43 (m, 2H), 7.40-7.32 (m, 3H), 6.44 (br d, 1H), 5.69 (dt, 1H), 3.03 (t, 2H), 2.40-2.31 (m, 2H), 1.69-1.59 (m, 2H), 1.53-1.32 (m, 4H).

### Example 122

### 4-((1E)-8-(1,3-oxazol-2-yl)-8-oxo-1-octenyl)benzonitrile

### Example 122A

### ethyl (7E)-8-(4-cyanophenyl)-7-octenoate

The desired product was prepared by substituting 4-cyanobenzaldehyde for 4-phenylbenzaldehyde in Example 87A.

### Example 122B

### (7E)-8-(4-cyanophenyl)-7-octenoic acid

The desired product was prepared by substituting Example 122A for Example 1A in Example 1B.

### Example 122C

### (7E)-8-(4-cyanophenyl)-7-octenoyl chloride

The desired product was prepared by substituting Example 122B for Example 102B in Example 102C.

### Example 122D

### 4-((1E)-8-(1,3-oxazol-2-yl)-8-oxo-1-octenyl)benzonitrile

The desired product was prepared by substituting Example 122C for Example 92C in Example 92D.

MS (ESI(+)) m/e 295 (M+H)⁺;

¹H NMR (DMSO-d₆) δ 8.38 (s, 1H), 7.80 (d, 2H), 7.53 (s, 1H), 7.46 (d, 2H), 6.48 (br d, 1H), 5.89-5.79 (m, 1H), 3.01 (t, 2H), 2.34-2.27 (m, 2H), 1.67-1.56 (m, 2H), 1.51-1.30 (m, 4H).

### Example 123

### N⁹-(1,1′-biphenyl)-3-yl-2-oxononanediamide

A suspension of Example 116 (354mg, 0.96 mmol) in ethanol (5 mL) at room temperature was treated with concentrated N₄OH (1 mL), stirred for 2 hours, and filtered. The isolated solid was washed with ethanol and dried under vacuum with heating to provide 158 mg (47%) of the desired product.

MS (ESI(+)) m/e 353 (M+H)⁺;

¹H NMR (DMSO-d₆) δ 9.96 (s, 1H), 7.94-7.89 (m, 2H), 7.64-7.54 (m, 4H), 7.50-7.43 (m, 2H), 7.41-7.29 (m, 3H), 2.78 (t, 2H), 2.32 (t, 2H), 1.63-1.54 (m, 2H), 1.54-1.45 (m, 2H), 1.34-1.26 (m, 4H).

### Example 124

### N¹-methyl-2-oxo-N⁹-(4-phenyl-1,3-thiazol-2-yl)nonanediamide

### Example 124A

### benzyl 7-hydroxyheptanoate

A solution of methyl 6-hydroxyhexanoate (5.0 g, 31.0 mmol, prepared according to the procedure described in Syn.Comm. 1991, 21 1075) in THF (11 mL) was treated with 2M LiOH (16 mL), heated to 60° C. for 4 hours, heated to 85° C. for 2 hours, and concentrated under nitrogen. The crude product was suspended in DMF (100 mL), treated with NaHCO₃(5.26 g, 6.3 mmol) and a solution of benzyl bromide (9.3 mL, 78 mmol) in DMF (50 mL), stirred for 18 hours, poured into water, and exctracted three times with ethyl acetate. The combined extracts were dried (Na₂SO₄), filtered, and concentrated. The concentrate was purified by flash column chromatography on silica gel with 10% ethyl acetate/hexanes to 30% ethyl acetate/hexanes to provide 4.43g (60%) of the desired product.

MS (ESI(+)) m/e 237 (M+H)⁺.

### benzyl 7-oxoheptanoate

### Example 124B

The desired product was prepared by substituting Example 124A for Example 91A in Example 91B.

### Example 124C

### 9-benzyl 1-methyl 2-oxononanedioate

The desired product was prepared by substituting Example 124B for Example 91B in Example 91C.

### Example 124D

### benzyl 9-(methylamino)-8,9-dioxononanoate

The desired product was prepared by substituting Example 124C for Example 118 in Example 119.

MS (ESI(+)) m/e 306 (M+H)⁺.

### Example 124E

### 9-(methylamino)-8,9-dioxononanoic acid

A solution of Example 124D (0.89 g, 2.91 mmol) and 10% Pd/C (95 mg) in methanol (15 mL) was stirred for 1 hour under a hydrogen atmosphere, filtered through diatomaceous earth (Celite®), and concentrated to provide 0.56g (89%) of the desired product.

MS (ESI(+)) m/e 216 (M+H)⁺.

### Example 124F

### N¹-methyl-2-oxo-N⁹-(4-phenyl-1,3-thiazol-2-yl)nonanediamide

The desired product was prepared by substituting Example 124E and 4-phenyl-1,3-thiazol-2-amine for Example 1B and 4-aminopyridine, respectively, in Example 6.

MS (APCI(+)) m/e 374 (M+H)⁺;

¹H NMR (DMSO-d₆) δ 12.20 (s, 1H), 8.53-8.74 (br m, 1H), 7.89 (d, 2H), 7.59 (s, 1H), 7.43 (t, 2H), 7.34-7.29 (m, 1H), 2.80 (t, 2H), 2.64 (d, 3H), 2.44 (t, 2H), 1.65-1.55 (m, 2H), 1.55-1.45 (m, 2H), 1.32-1.26 (m, 4H).

### Example 125

### N¹-methyl-2-oxo-N 9-(4-phenoxyphenyl)nonanediamide

The desired product was prepared by substituting Example 124E and 4-phenoxyaniline for Example 1B and 4-aminopyridine, respectively, in Example 6.

MS (ESI(+)) m/e 383 (M+H)⁺;

¹H NMR (DMSO-d₆) δ 9.87 (s, 1H), 8.52-8.47 (br m, 1H), 7.59 (d, 2H), 7.48-7.43 (m, 2H), 7.11-7.06 (m, 1H), 6.98-7.93 (m, 4H), 2.80 (t, 2H), 2.64 (d, 3H), 2.28 (t, 2H), 1.62-1.45 (m, 4H), 1.32-1.26 (m, 4H).

### Example 126

N⁹-(4,5-diphenyl-1,3-thiazol-2-yl)-N -methyl-2-oxononanediamide

The desired product was prepared by substituting Example 124E and 4,5-diphenyl-1,3-thiazol-2-amine for Example 1B and 4-aminopyridine, respectively, in Example 6.

MS (ESI(+)) m/e 450 (M+H)⁺;

¹H NMR (DMSO-d₆) δ 12.26 (s, 1H), 8.52-8.47 (br m, 1H), 7.44-7.29 (m, 10H), 2.80 (t, 2H), 2.64 (d, 3H), 2.45 (t, 2H), 1.64-1.56 (m, 2H), 1.54-1.44 (m, 2H), 1.33-1.26 (m, 4H).

### Example 127

### N⁹-(4-(3-methoxyphenyl)-1,3-thiazol-2-yl)-N -methyl-2-oxononanediamide

The desired product was prepared by substituting Example 124E and 4-(3-methoxyphenyl)-1,3-thiazol-2-amine for Example 1B and 4-aminopyridine, respectively, in Example 6.

MS (ESI(+)) m/e 404 (M+H)⁺;

¹H NMR (DMSO-d₆) δ 12.19 (s, 1H), 8.53-8.47 (br m, 1H), 7.62 (s, 1H), 7.49-7.44 (m, 2H), 7.38-7.27 (m, 1H), 6.91-6.84 (m, 1H), 3.80 (s, 3H), 2.80 (t, 2H), 2.64 (d, 3H), 2.44 (t, 2H), 1.64-1.55 (m, 2H), 1.53-1.43 (m, 2H), 1.32-1.23 (m, 4H).

### Example 128

### N⁹-(4-(2-methoxyphenyl)-1,3-thiazol-2-yl)-N¹-methyl-2-oxononanediamide

The desired product was prepared by substituting Example 124E and 4-(2-methoxyphenyl)-1,3-thiazol-2-amine for Example 1B and 4-aminopyridine, respectively, in Example 6.

MS (ESI(+)) m/e 404 (M+H)⁺;

¹H NMR (DMSO-d₆) δ 12.12 (s, 1H), 8.53-8.47 (br m, 1H), 8.05 (dd, 1H), 7.62 (s, 1H), 7.33-7.37 (m, 1H), 7.14-7.11 (m, 1H), 7.05-7.00 (m, 1H), 3.91 (s, 3H), 2.80 (t, 2H), 2.64 (d, 3H), 2.44 (t, 2H), 1.64-1.56 (m, 2H), 1.56-1.44 (m, 2H), 1.32-1.25 (m, 4H).

### Example 129

### N¹-methyl-N⁹-(5-methyl-4-phenyl-1,3-thiazol-2-yl)-2-oxononanediarnide

The desired product was prepared by substituting Example 124E and 5-methyl-4-phenyl-1,3-thiazol-2-amine for Example 1B and 4-aminopyridine, respectively, in Example 6.

MS (ESI(+)) m/e 388 (M+H)⁺;

¹H NMR (DMSO-d₆) δ 12.03 (s, 1H), 8.52-8.47 (br m, 1H), 7.54-7.51 (m, 2H), 7.44 (t, 2H), 7.34-7.31 (m, 1H), 2.79 (t, 2H), 2.64 (d, 3H), 2.46 (s, 3H), 2.41 (t, 2H), 1.62-1.54 (m, 2H), 1.54-1.44 (m, 2H), 1.30-1.25 (m, 4H).

### Example 130

### N¹-methyl-2-oxo-N⁹-(4-(trifluoromethoxy)(1,1′-biphenyl)-3-yl)nonanediamide

The desired product was prepared by substituting Example 124E and 4-(4-trifluoromethoxyphenoxy)aniline for Example 1B and 4-aminopyridine, respectively, in Example 6.

MS (ESI(+)) m/e 451 (M+H)⁺;

¹H NMR (DMSO-d₆) δ 9.98 (s, 1H), 8.53-8.48 (br m, 1H), 8.05-8.03 (m, 1H), 7.72 (d, 2H), 7.59-7.56 (m, 1H), 7.48-7.30 (m, 4H), 2.80 (t, 2H), 2.64 (d, 3H), 2.32 (t, 2H), 1.64-1.55 (m, 2H), 1.55-1.46 (m, 2H), 1.33-1.28 (m, 4H).

### Example 131

### N⁹-(4-(4-chlorophenoxy)phenyl)-N -methyl-2-oxononanediamide

The desired product was prepared by substituting Example 124E and 4-(4-chlorophenoxy)aniline for Example 1B and 4-aminopyridine, respectively, in Example 6.

MS (ESI(+)) m/e 417 (M+H)⁺;

¹H NMR (DMSO-d₆) δ 9.89 (s, 1H), 8.53-8.46 (br m, 1H), 7.61 (d, 2H), 7.40 (d, 2H), 7.01-6.95 (m, 4H), 2.80 (t, 2H), 2.64 (d, 3H), 2.28 (t, 2H), 1.62-1.45 (m, 4H), 1.33-1.26 (m, 4H).

### Example 132

### N⁹-(4-methoxy(1,1′-biphenyl)-3-yl)-N¹-methyl-2-oxononanediamide

The desired product was prepared by substituting Example 124E and 4-methoxy(1,1′-biphenyl)-3-amine for Example 1B and 4-aminopyridine, respectively, in Example 6.

MS (ESI(+)) m/e 397 (M+H)⁺;

¹H NMR (DMSO-d₆) δ 9.11 (s, 1H), 8.53-8.47 (br m, 1H), 8.30-8.28 (br s, 1H), 7.56 (d, 2H), 7.46-7.41 (m, 2H), 7.38-7.29 (m, 2H), 7.12 (d, 1H), 3.87 (s, 3H), 2.81 (t, 2H), 2.64 (d, 3H), 2.43-2.36 (m, 2H), 1.63-1.48 (m, 4H), 1.34-1.27 (m, 4H).

### Example 133

### N⁹-(4′-cyano(ll′-biphenyl)-3-yl)-N¹-methyl-2-oxononanediamide

The desired product was prepared by substituting Example 124E and 3′-amino(1,1′-biphenyl)-4-carbonitrile for Example lB and 4-aminopyridine, respectively, in Example 6.

MS (ESI(+)) m/e 392 (M+H)⁺;

¹H NMR (DMSO-d₆) δ 10.01 (s, 1H), 8.52-8.47 (br m, 1H), 8.02-8.00 (br m, 1H), 7.93 (d, 2H), 7.80 (d, 2H), 7.63-7.59 (m, 1H), 7.46-7.38 (m, 2H), 2.80 (t, 2H), 2.64 (d, 3H), 2.32 (t, 2H), 1.64-1.45 (m, 4H), 1.33-1.28 (m, 4H).

### Example 134

### N⁹-(4-bromophenyl)-N¹-methyl-2-oxononanediamide

The desired product was prepared by substituting Example 124E and 4-bromoaniline for Example 1B and 4-aminopyridine, respectively, in Example 6.

MS (ESI(+)) m/e 368 (M+H)⁺;

¹H NMR (DMSO-d₆) δ 9.97 (s, 1H), 8.52-8.47 (br s, 1H), 7.55 (d, 2H), 7.46 (d, 2H), 2.79 (t, 2H), 2.64 (d, 3H), 2.28 (t, 2H), 1.61-1.44 (m, 4H), 1.32-1.24 (m, 4H).

### Example 135

### N⁹-(6-methoxy(1,1′-biphenyl)-3-yl)-N¹-methyl-2-oxononanediamide

The desired product was prepared by substituting Example 124E and 6-methoxy(1,1′-biphenyl)-3-amine for Example 1B and 4-aminopyridine, respectively, in Example 6.

MS (ESI(+)) m/e 397 (M+H)⁺;

¹H NMR (DMSO-d₆) δ 9.76 (s, 1H), 8.52-8.47 (br m, 1H), 7.54-7.51 (m, 2H), 7.45-7.36 (m, 4H), 7.34-7.29 (m, 1H), 7.03 (d, 1H), 3.72 (s, 3H), 2.79 (t, 2H), 2.64 (d, 3H), 2.26 (t, 2H), 1.61-1.45 (m, 4H), 1.32-1.25 (m, 4H).

### Example 136

### N⁹-(1,1′-biphenyl)-4-yl-N¹-methyl-2-oxononanediamide

The desired product was prepared by substituting Example 124E and (1,1′-biphenyl)-4-amine for Example 1B and 4-aminopyridine, respectively, in Example 6.

MS (ESI(+)) m/e 367 (M+H)⁺;

¹H NMR (DMSO-d₆) δ 9.94 (s, 1H), 8.52-8.47 (br m, 1H), 7.70-7.58 (m, 6H), 7.44 (t, 2H), 7.32 (t, 1H), 2.80 (t, 2H), 2.64 (d, 3H), 2.32 (t, 2H), 1.63-1.56 (m, 2H), 1.55-1.46 (m, 2H), 1.33-1.27 (m, 4H).

### Example 137

### N⁹-(3,4-dichlorophenyl)-N¹-methyl-2-oxononanediamide

The desired product was prepared by substituting Example 124E and 3,4-dichloroaniline for Example 1B and 4-aminopyridine, respectively, in Example 6.

MS (ESI(+)) m/e 359 (M+H)⁺;

¹H NMR (DMSO-d₆) δ 10.15 (s, 1H), 8.53-8.47 (br s, 1H), 7.99 (d, 1H), 7.54 (d, 1H), 7.47 (dd, 1H), 2.79 (t, 2H), 2.64 (d, 3H), 2.30 (t, 2H), 1.62-1.44 (m, 4H), 1.32-1.25 (m, 4H).

### Example 138

### N⁹-methyl-2-oxo-N¹-(4-(trifluoromethyl)phenyl)nonanediamide

The desired product was prepared by substituting Example 124E and 4-trifluoromethylaniline for Example 1B and 4-aminopyridine, respectively, in Example 6.

MS (ESI(+)) m/e 359 (M+H)⁺;

¹H NMR (DMSO-d₆) δ 10.21 (s, 1H), 8.52-8.47 (br m, 1H), 7.80 (d, 2H), 7.64 (d, 2H), 2.79 (t, 2H), 2.64 (d, 3H), 2.33 (t, 2H), 1.63-1.44 (m, 4H), 1.33-1.26 (m, 4H).

### Example 139

N⁹-(3-cyanophenyl)-N¹-methyl-2-oxononanediamide

The desired product was prepared by substituting Example 124E and 3-cyanoaniline for Example 1B and 4-aminopyridine, respectively, in Example 6.

MS (ESI(+)) m/e 316 (M+H)⁺;

¹H NMR (DMSO-d₆) δ 10.20 (s, 1H), 8.53-8.46 (br m, 1H), 8.10-8.08 (br m, 1H), 7.80-7.76 (m, 1H), 7.51-7.47 (m, 2H), 2.79 (t, 2H), 2.64 (d, 3H), 2.32 (t, 2H), 1.63-1.44 (m, 4H), 1.32-1.26 (m, 4H).

### Example 140

### N⁹-(4-(4-methoxyphenyl)-1,3-thiazol-2-yl)-N¹-methyl-2-oxononanediamide

The desired product was prepared by substituting Example 124E and 4-(4-methoxyphenyl)-1,3-thiazol-2-amine for Example 1B and 4-aminopyridine, respectively, in Example 6.

MS (ESI(+)) m/e 404 (M+H)⁺;

¹H NMR (DMSO-d₆) δ 12.15 (s, 1H), 8.53-8.48 (br m, 1H), 7.81 (d, 2H), 7.42 (s, 1H), 6.98 (d, 2H), 3.79 (s, 3H), 2.80 (t, 2H), 2.64 (d, 3H), 2.43 (t, 2H), 1.64-1.54 (m, 2H), 1.54-1.44 (m, 4H), 1.31-1.26 (m, 4H).

Following Scheme 12 and the Examples described above, the following compound can be prepared:

### Example 141

### methyl 8-((4′-cyano(1,1′-biphenyl)-4-yl)oxy)-3-hydroxy-2-oxooctanoate

It will be evident to one skilled in the art that the present invention is not limited to the forgoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims and therefore intended to be embraced therein.

What is claimed is:

1. A compound of formula (I) 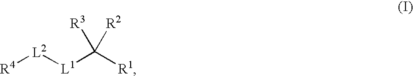
or a therapeutically acceptable salt thereof, wherein
L¹ is selected from the group consisting of alkenylene, alkylene, alkynylene, cycloalkylene, heteroalkylene, and -(alkylene)C(O)N(R⁵)(alkylene)-;
wherein each group is drawn with its left-hand end being the end which attaches to L², and its right-hand end being the end which attaches to the carbon substituted with R¹, R², and R³;
L² is selected from the group consisting of C₂ alkenylene, —O—, —S—, —SO₂—, —OC(O)NR⁵—, —N(R⁶)C(O)—, and —C(O)N(R )—,
wherein each group is drawn with its left-hand end being the end which attaches to R⁴, and its right-hand end being the end which attaches to L¹;
R¹ is selected from the group consisting of alkanoyl, alkoxycarbonyl, aminocarbonyl, carboxy, perfluoroalkyl, and heterocycle, wherein the heterocycle is selected from the group consisting of oxazolyl, dihydrooxazolyl, and oxadiazole;
R² and R³ are hydroxy; or
R² and R³ together are oxo;
R⁴ is selected from the group consisting of alkoxyalkyl, alkyl, aryl, arylalkyl, cycloalkyl, (cycloalkyl)alkyl, heterocycle, and (heterocycle)alkyl; and
R⁵ and R⁶ are independently selected from the group consisting of hydrogen, alkyl, aryl, and arylalkyl; or
R⁴ and R⁶, together with the nitrogen atom to which they are attached, form a heterocycle selected from the group consisting of morpholinyl, piperazinyl, piperidinyl, and thiomorpholinyl; wherein the morpholinyl, the piperazinyl, the piperidinyl, and the thiomorpholinyl can be optionally substituted with one, two, or three substituents independently selected from the group consisting of alkyl and spiroheterocycle.

2. A compound according to claim 1 wherein R¹ is heterocycle, wherein the heterocycle is oxazolyl or dihydrooxazolyl.

3. A compound according to claim 2 wherein L¹ is C₅-C₇ alkylene.

4. A compound according to claim 3 selected from the group consisting of
7-((1,1′-biphenyl)-3-yloxy)-1-(1,3-oxazol-2-yl)-1-heptanone,
8-(1,3-oxazol-2-yl)-8-oxo-N-phenyloctanamide,
N-(1,1′-biphenyl)-3-yl-8-(1,3-oxazol-2-yl)-8-oxooctanamide,
N-(4-chlorophenyl)-8-(1,3-oxazol-2-yl)-8-oxooctanamide,
8-(1,3-oxazol-2-yl)-8-oxo-N-(4-phenoxyphenyl)octanamide,
8-(1,3-oxazol-2-yl)-8-oxo-N-(2-pyridinyl)octanamide,
1-(1,3-oxazol-2-yl)-7-(phenylsulfanyl)-1-heptanone,
1-(1,3-oxazol-2-yl)-7-(phenylsulfonyl)-1-heptanone,
7-(2-naphthylsulfanyl)-1-(1,3-oxazol-2-yl)-1-heptanone,
7-(2-naphthylsulfonyl)-1-(1,3-oxazol-2-yl)-1-heptanone,
N-(6-(1,3-oxazol-2-yl)-6-oxohexyl)-1H-indole-2-carboxamide,
7-((1,1′-biphenyl)-4-yloxy)-1-(4,5-dihydro-1,3-oxazol-2-yl)-1-heptanone,
(7E)-8-(1,1′-biphenyl)4-yl-1-(1 ,3-oxazol-2-yl)-7-octen-1-one, and
4-((1E)-8-(1,3-oxazol-2-yl)-8-oxo-1-octenyl)benzonitrile.

5. A compound according to claim 1 wherein R¹ is selected from the group consisting of alkoxycarbonyl and carboxy.

6. A compound according to claim 5 wherein L¹ is C₅-C₇ alkylene.

7. A compound according to claim 6 selected from the group consisting of
methyl 8-((1,1′-biphenyl)-4-yloxy)-2-oxooctanoate,
8-((1,1′-biphenyl)-4-yloxy)-2-oxooctanoic acid,
ethyl 7-((1,1′-biphenyl)-4-yloxy)-2-oxoheptanoate,
ethyl 7-((3-bromo(1,1′-biphenyl)-4-yl)oxy)-2-oxoheptanoate,
methyl 7-((1,1′-biphenyl)-4-yloxy)-2-oxoheptanoate,
methyl 9-((1,1′-biphenyl)-3-ylamino)-2,9-dioxononanoate,
methyl 9-anilino-2,9-dioxononanoate,
methyl 8-((4′-cyano(1,1′-biphenyl)-4-yl)oxy)-2-oxooctanoate, and
methyl 8-((4′-cyano(1,1′-biphenyl)-4-yl)oxy)-3-hydroxy-2-oxooctanoate.

8. A compound according to claim 1 wherein R¹ is alkanoyl.

9. A compound according to claim 8 wherein L¹ is C₅-C₇ alkylene.

10. A compound according to claim 9 selected from the group consisting of
9-((1,1′-biphenyl)-4-yloxy)-2,3-nonanedione, and
N-(1,1′-biphenyl)-4-yl-7,8-dioxononanamide.

11. A compound according to claim 1 wherein R¹ is aminocarbonyl.

12. A compound according to claim 11 wherein L¹ is C₅-C₇ alkylene.

13. A compound according to claim 12 selected from the group consisting of
8-((1,1′-biphenyl)-4-yloxy)-N-methyl-2-oxooctanamide,
N-methyl-8-(2-naphthylsulfanyl)-2-oxooctanamide,
N-methyl-8-(2-naphthylsulfonyl)-2-oxooctanamide,
8-((1,1′-biphenyl)-4-ylsulfanyl)-N-methyl-2-oxooctanamide,
8-((1,1′-biphenyl)-4-ylsulfonyl)-N-methyl-2-oxooctanamide,
8-((1,1′-biphenyl)-4-ylsulfonyl)-N-methyl-2-oxooctanamide,
8-((4′-cyano(l,l′-biphenyl)-4-yl)oxy)-N-methyl-2-oxooctanamide,
N⁹-(1,1′-biphenyl)-3-yl-Nl-methyl-2-oxononanediamide,
N⁹-(1,1′-biphenyl)-3-yl-2-oxononanediamide,
N⁹-methyl-2-oxo-N -(4-phenyl-1,3-thiazol-2-yl)nonanediamide,
N⁹-methyl-2-oxo-N -(4-phenoxyphenyl)nonanediamide,
N⁹-(4,5-diphenyl-1,3-thiazol-2-yl)-N¹-methyl-2-oxononanediamide,
N⁹-(4-(3-methoxyphenyl)-1,3-thiazol-2-yl)-N¹-methyl-2-oxononanediamide,
N⁹-(4-(2-methoxyphenyl)-1,3-thiazol-2-yl)-N¹-methyl-2-oxononanediamide,
N¹-methyl-N⁹-(5-methyl-4-phenyl-1,3-thiazol-2-yl)-2-oxononanediamide,
N¹-methyl-2-oxo-N -(4′-(trifluoromethoxy)(1,1′-biphenyl)-3-yl)nonanediamide,
N⁹-(4-(4-chlorophenoxy)phenyl)-N¹-methyl-2-oxononanediamide,
N⁹-(4-methoxy(1,1′-biphenyl)-3-yl)-N¹-methyl-2-oxononanediamide,
N⁹-(4′-cyano(1,1′-biphenyl)-3-yl)-N¹-methyl-2-oxononanediamide,
N⁹-(4-bromophenyl)-N¹-methyl-2-oxononanediamide,
N⁹-(6-methoxy(1,1biphenyl)-3-yl)-N¹-methyl-2-oxononanediamide,
N⁹-(6methoxy(1,1′-biphenyl)-4-yl-N¹-methyl-2-oxononanediamide,
N⁹-(3,4-dichlorophenyl)-N¹-methyl-2-oxononanediamide,
N¹-methyl-2-oxo-N -(4-(trifluoromethyl)phenyl)nonanediamide,
N⁹-(3-cyanophenyl)-N¹-methyl-2-oxononanediamide, and
N⁹-(4-(4-methoxyphenyl)-1,3-thiazol-2-yl)-N¹-methyl-2-oxononanediamide.

14. A compound according to claim 1 wherein R¹ is perfluoroalkyl.

15. A compound according to claim 14 wherein L¹ is selected from the group consisting of C₆ alkenylene, C₆ alkynylene, cycloalkylene, and -(alkylene)C(O)N(R 5)(alkylene)-.

16. A compound according to claim 15 selected from the group consisting of 8-((1,1′-biphenyl)-4-yloxy)-1,1,1-trifluoro-3-octyn-2-one, (3E)-8-((1,1′-biphenyl)-4-yloxy)-1,1,1-trifluoro-3-octen-2-one, 1-(2-(4-((1,1′-biphenyl)-4-yloxy)butyl)cyclopropyl)-2,2,2-trifluoroethanone, and 4-((1,1′-biphenyl)-4-yloxy)-N-(3,3,3-trifluoro-2-oxopropyl)butanamide.

17. A compound according to claim 14 wherein L¹ is C₅-C₇ alkylene.

18. A compound according to claim 17 wherein L² is C₂ alkenylene.

19. A compound according to claim 18 which is (8E)-9-(1,1′-biphenyl)-4-yl-1,1,1-trifluoro-8-nonen-2-one.

20. A compound according to claim 17 wherein L² is —OC(O)N(R⁵)—.

21. A compound according to claim 20 which is 9H-fluoren-9-ylmethyl 7,7,7-trifluoro-6-oxoheptylcarbamate.

22. A compound according to claim 17 wherein L² is —O—.

23. A compound according to claim 22 selected from the group consisting of
8-((1,1′-biphenyl)-4-yloxy)-1,1,1-trifluoro-2-octanone,
4′-((8,8,8-trifluoro-7-oxooctyl)oxy)(1,1′-biphenyl)-4-carbonitrile,
9-((1,1′-biphenyl)-4-yloxy)-1,1,1-trifluoro-2-nonanone,
7-((1,1′-biphenyl)-4-yloxy)-1,1,1-trifluoro-2-heptanone,
8-(3-bromophenoxy)-1,1,1-trifluoro-2-octanone,
1,1,1-trifluoro-8-(3-(4-pyridinyl)phenoxy)-2-octanone,
8-(4-bromophenoxy)-1,1,1-trifluoro-2-octanone,
1,1,1-trifluoro-8-(4-phenoxyphenoxy)-2-octanone, and
8-((1,1′-biphenyl)-3-yloxy)-1,1,1-trifluoro-2-octanone.

24. A compound according to claim 17 wherein L² is —N(R⁶)C(O)—.

25. A compound according to claim 24 wherein R⁴ is selected from the group consisting of alkoxyalkyl and alkyl.

26. A compound according to claim 25 selected from the group consisting of 9,9,9-trifluoro-N-(3-methoxypropyl)-8-oxononanamide, and 9,9,9-trifluoro-N-isopentyl-8-oxononanamide.

27. A compound according to claim 24 wherein R⁴ is aryl.

28. A compound according to claim 27 selected from the group consisting of
9,9,9-trifluoro-8-oxo-N-phenylnonanamide,
9,9,9-trifluoro-N-(4-methoxyphenyl)-8-oxononanamide,
9,9,9-trifluoro-N-(3-methoxyphenyl)-8-oxononanamide,
9,9,9-trifluoro-N-(2-methoxyphenyl)-8-oxononanamide,
N-(4-(dimethylarino)phenyl)-9,9,9-trifluoro-8-oxononanamide,
N-(1,1′-biphen yl)-4-yl-9,9,9-trifluoro-8-oxono nana mide,
N-(1,1′-biphenyl)-3-yl-9,9,9-trifluoro-8-oxononanamide,
N-(1,1′-biphenyl)-2-yl-9,9,9-trifluoro-8-oxononanamide,
N-(4-cyclohexylphenyl)-9,9,9-trifluoro-8-oxononanamide,
9,9,9-trifluoro-8-oxo-N-(4-(1-piperidinyl)phenyl)nonanamide,
9,9,9-trifluoro-N-(4-(4-morpholinyl)phenyl)-8-oxononanamide,
9,9,9-trifluoro-N-(4-hydroxyphenyl)-8-oxononanamide,
9,9,9-trifluoro-N-(4-fluorophenyl)-8-oxononan amide,
N-(3-cyanophenyl)-9,9,9-trifluoro-8-oxononanamide,
N-(4-chlorophenyl)-9,9,9-trifluoro-8-oxononanamide,
N-(4-acetylphenyl)-9,9,9-trifluoro-8-oxononanamide,
9,9,9-trifluoro-8-oxo-N-(4-(trifluoromethyl)phenyl)nonanamide,
N-(3,4-dichlorophenyl)-9,9,9-trifluoro-8-oxononanamide,
N-(4-bromophenyl)-9,9,9-trifluoro-8-oxonon anamide,
N-(4-benzylphenyl)-9,9,9-trifluoro-8-oxononanamide,
9,9,9-trifluoro-8-oxo-N-(4-phenoxyphenyl)nonanamide,
9,9,9-trifluoro-8,8-dihydroxy-N-(9-oxo-9H-fluoren-2-yl)nonanamide,
N-(4-(benzyloxy)phenyl)-9,9,9-trifluoro-8-oxononanamide,
N-(4′-cyano(1,1′-biphenyl)-3-yl)-9,9,9-trifluoro-8-oxononanamide,
N-(3-(benzyloxy)phenyl)-9,9,9-trifluoro-8-oxononanamide,
9,9,9-trifluoro-8-oxo-N-(3-phenoxyphenyl)nonanamide,
N-(3-benzoylphenyl)-9,9,9-trifluoro-8-oxononanamide,
9,9,9-trifluoro-8-oxo-N-(4′-(trifluoromethoxy)(1,1′-biphenyl)-3-yl)nonanamide,
9,9,9-trifluoro-8-oxo-N-(3-(3-pyridinyl)phenyl)nonanamide,
9,9,9-trifluoro-N-(4′-(methylsulfanyl)(1,1′-biphenyl)-3-yl)-8-oxononanamide,
N-(3 ′-amino(1,1′-biphenyl)-3-yl)-9,9,9-trifluoro-8-oxononanamide,
9,9,9-trifluoro-N-(4′-(methylsulfonyl)(1,1′-biphenyl)-3-yl)-8-oxononanamide,
N-(4′-cyano(1,1′-biphenyl)-3-yl)-8,8,8-trifluoro-7-oxooctanamide,
N-(3-cyanophenyl)-8,8,8-trifluoro-7-oxooctanamide,
8,8,8-trifluoro-7-oxo-N-(4-phenoxyphenyl)octanamide,
9,9,9-trifluoro-N-(3-methylphenyl)-8-oxononanamide,
9,9,9-trifluoro-N-(4-methylphenyl)-8-oxononanamide,
N-(4-aminophenyl)-9,9,9-trifluoro-8-oxononanamide,
9,9,9-trifluoro-8-oxo-N-(3-(trifluoromethoxy)phenyl)nonanamide,
N-(2,4-dimethylphenyl)-9,9,9-trifluoro-8-oxononanamide,
N-(3,4-dimethylphenyl)-9,9,9-trifluoro-8-oxononanamide,
N-(3,5-dimethylphenyl)-9,9,9-trifluoro-8-oxononanamide,
N-(2,4-dimethoxyphenyl)-9,9,9-trifluoro-8-oxononanamide,
N-(3,5-dimethoxyphenyl)-9,9,9-trifluoro-8-oxononanamide, and
9,9,9-trifluoro-8-oxo-N-(3,4,5-trimethoxyphenyl)nonanamide.

29. A compound according to claim 24 wherein R⁴ is arylalkyl.

30. A compound according to claim 29 selected from the group consisting of
N-benzyl-9,9,9-trifluoro-8-oxononanamide,
9,9,9-trifluoro-8-oxo-N-(2-phenyleth yl)nonanamide,
9,9,9-trifluoro-8-oxo-N-(3-phenylpropyl)nonanamide,
N-((1S)-1-benzyl-2-(m ethylamino)-2-oxoethyl)-9,9,9-trifluoro-8-oxononanamide,
N-benzhydryl-9,9,9-trifluoro-8-oxononanamide,
9,9,9-trifluoro-N-(4-fluorobenzyl)-8-oxononanamide,
9,9,9-trifluoro-N-(3-fluorobenzyl)-8-oxononanamide,
9,9,9-trifluoro-N-(3-chlorobenzyl)-8-oxononanamide,
N-(4-bromobenzyl)-9,9,9-trifluoro-8-oxononanamide,
9,9,9-trifluoro-8-oxo-N-(3-(trifluoromethoxy)benzyl)nonanamide,
9,9,9-trifluoro-8-oxo-N-(3-(trifluoromethyl)benzyl)nonanamide,
N-(3,5-dimethoxybenzyl)-9,9,9-trifluoro-8-oxononanamide,
N-(2,5-dimethoxybenzyl)-9,9,9-trifluoro-8-oxononanamide,
N-(3,4-dichlorobenzyl)-9,9,9-trifluoro-8-oxononanamide, and
N-(4-(dimethylamino)benzyl)-9,9,9-trifluoro-8-oxononanamide.

31. A compound according to claim 24 wherein R⁴ is selected from the group consisting of cycloalkyl, heterocycle, and (heterocycle)alkyl.

32. A compound according to claim 31 selected from the group consisting of
9,9,9-trifluoro-8-oxo-N-(4-pyridinyl)nonanamide,
9,9,9-trifluoro-8-oxo-N-(3-pyridinylmethyl)nonanamide,
N-(1,3-benzodioxol-5-yl)-9,9,9-trifluoro-8-oxononanamide,
9,9,9-trifluoro-8-oxo-N-(3-pyridinyl)nonanamide,
N-cyclohexyl-9,9,9-trifluoro-8,8-dihydroxynonanamide,
N-(2-adamantyl)-9,9,9-trifluoro-8-oxononanamide,
9,9,9-trifluoro-8-oxo-N-(4-phenyl-1,3-thiazol-2-yl)nonanamide, and
N-(1,3-benzodioxol-5-ylmethyl)-9,9,9-trifluoro-8-oxononanamide.

33. A compound according to claim 24 wherein R⁴ and R⁶, together with the nitrogen atom to which they are attached, form a ring selected from the group consisting of morpholinyl, piperazinyl, piperidinyl, and thiomorpholinyl.

34. A compound according to claim 33 which is 9-(1 ,4-dioxa-8-azaspiro(4.5)dec-8-yl)-1,1, 1-trifluoro-9-oxo-2,2-nonanediol.

35. A pharmaceutical composition comprising a compound of claim 1, or a therapeutically acceptable salt thereof, in combination with a pharmaceutically acceptable carrier.

36. A method of inhibiting histone deacetylase in a patient in recognized need of such treatment comprising administering to the patient a therapeutically acceptable amount of a compound of claim 1, or a therapeutically acceptable salt thereof.

37. A compound selected from the group consisting of
9,9,9-trifluoro-8-oxo-N-phenylnonanamide,
8-((1,1′-biphenyl)-4-yloxy)-1,1,1-trifluoro-2-octanone,
4′-((8 ,8, 8-trifluoro-7-oxooctyl)oxy)(1,1′-biphenyl)-4-carbonitrile,
9-((1,1′-biphenyl)-4-yloxy)-1,1,1-trifluoro-2-nonanone,
7-((1,1′-biphenyl)-4-yloxy)-1,1,1-trifluoro-2-heptanone,
9,9,9-trifluoro-8-oxo-N-(4-pyridinyl)nonanamide,
N-benzyl-9 ,9,9-trifluoro-8-oxononanamide,
9,9,9-trifluoro-8-oxo-N-(3-pyridinylmethyl)nonanamide,
9,9,9-trifluoro-8-oxo-N-(2-phenylethyl)nonanamide,
9,9,9-trifluoro-N-(4-methoxyphenyl)-8-oxononanamide,
9,9,9-trifluoro-N-(3-methoxyphenyl)-8-oxononanamide,
9,9,9-trifluoro-N-(2-methoxyphenyl)-8-oxononanamide,
9,9,9-trifluoro-8-oxo-N-(3-phenylpropyl)nonanamide,
N-(4-(dimethylamino)phenyl)-9,9,9-trifluoro-8-oxononanamide,
N-(1,3-benzodioxol-5-yl)-9,9,9-trifluoro-8-oxononanamide,
9-(1,4-dioxa-8-azaspiro(4.5)dec-8-yl)-1,1,1-trifluoro-9-oxo-2,2-nonanediol,
N-(1,1′-biphenyl)-4-yl-9,9,9-trifluoro-8-oxononanamide,
N-(1,1′-biphenyl)-3-yl-9,9,9-trifluoro-8-oxononanamide,
N-(1,1′-biphenyl)-2-yl-9,9,9-trifluoro-8-oxononanamide,
N-(4-cyclohexylphenyl)-9,9,9-trifluoro-8-oxononanamide,
9,9,9-trifluoro-8-oxo-N-(4-(1-piperidinyl)phenyl)nonanamide,
9,9,9-trifluoro-N-(4-(4-morpholinyl)phenyl)-8-oxononanamide,
N-((1S)-1-benzyl-2-(methylamino)-2-oxoethyl)-9,9,9-trifluoro-8-oxononanamide,
N-benzhydryl-9,9,9-trifluoro-8-oxononanamide, 9,9,9-trifluoro-8-oxo-N-(3-pyridinyl)nonanamide,
N-cyclohexyl-9,9,9-trifluoro-8,8-dihydroxynonanamide,
9,9,9-trifluoro-N-(4-hydroxyphenyl)-8-oxononanamide,
9,9,9-trifluoro-N-(4-fluorophenyl)-8-oxononanamide,
N-(3-cyanophenyl)-9,9,9-trifluoro-8-oxononanamide,
N-(4-chlorophenyl)-9,9,9-trifluoro-8-oxononanamide,
N-(4-acetylphenyl)-9,9,9-trifluoro-8-oxononanamide,
N-(2-adamantyl)-9,9,9-trifluoro-8-oxononanamide,
9,9,9-trifluoro-8-oxo-N-(4-(trifluoromethyl)phenyl)nonanamide,
N-(3,4-dichlorophenyl)-9,9,9-trifluoro-8-oxononanamide,
N-(4-bromophenyl)-9,9,9-trifluoro-8-oxononanamide,
N-(4-benzylphenyl)-9,9,9-trifluoro-8-oxononanamide,
9,9,9-trifluoro-8-oxo-N-(4-phenoxyphenyl)nonanamide,
9,9,9-trifluoro-8,8-dihydroxy-N-(9-oxo-9H-fluoren-2-yl)nonanamide,
N-(4-(benzyloxy)phenyl)-9,9,9-trifluoro-8-oxononanamide,
9,9,9-trifluoro-N-(3-methoxypropyl)-8-oxononanamide,
9,9,9-trifluoro-N-isopentyl-8-oxononanamide,
N-(4 ′-cyano(1,1′-biphenyl)-3-yl)-9,9,9-trifluoro-8-oxononanamide,
N-(3-(benzyloxy)phenyl)-9,9,9-trifluoro-8-oxononanamide,
9,9,9-trifluoro-8-oxo-N-(3-phenoxyphenyl)nonanamide,
N-(3-benzoylphenyl)-9,9,9-trifluoro-8-oxononanamide,
9,9,9-trifluoro-8-oxo-N-(4-phenyl-1,3-thiazol-2-yl)nonanamide,
8-(3-bromophenoxy)-1,1,1-trifluoro-2-octanone,
1,1,1-trifluoro-8-(3-(4-pyridinyl)phenoxy)-2-octanone,
8-(4-bromophenoxy)-1,1,1-trifluoro-2-octanone,
1,1,1-trifluoro-8-(4-phenoxyphenoxy)-2-octanone,
8-((1,1′-biphenyl)-3-yloxy)-1,1,1-trifluoro-2-octanone,
9,9,9-trifluoro-8-oxo-N-(4′-(trifluoromethoxy)(,1′-biphenyl)-3-yl)nonanamide,
9,9,9-trifluoro-8-oxo-N-(3-(3-pyridinyl)phenyl)nonanamide,
9,9,9-trifluoro-N-(4′-(methylsulfanyl)(1,1′-biphenyl)-3-yl)-8-oxononanamide,
N-(3 ′-amino(1,1′-biphenyl)-3-yl)-9,9,9-trifluoro-8-oxononanamide,
9,9,9-trifluoro-N-(4′-(methylsulfonyl)(1,1′-biphenyl)-3-yl)-8-oxononanamide,
N-(4′-cyano(1,1′-biphenyl)-3-yl)-8 ,8,8-trifluoro-7-oxooctanamide,
N-(3-cyanophenyl)-8,8,8-trifluoro-7-oxooctanamide,
8,8,8-trifluoro-7-oxo-N-(4-phenoxyphenyl)octanamide,
9,9,9-trifluoro-N-(3-methylphenyl)-8-oxononanamide,
9,9,9-trifluoro-N-(4-methylphenyl)-8-oxononanamide,
N-(4-aminophenyl)-9,9,9-trifluoro-8-oxononanamide,
9,9,9-trifluoro-N-(4-fluorobenzyl)-8-oxononanarnide,
9,9,9-trifluoro-N-(3-fluorobenzyl)-8-oxononanamide,
9,9,9-trifluoro-N-(3-chlorobenzyl)-8-oxononanamide,
N-(4-bromobenzyl)-9,9,9-trifluoro-8-oxononanamide,
9,9,9-trifluoro-8-oxo-N-(3-(trifluoromethoxy)benzyl)nonanamide,
9,9,9-trifluoro-8-oxo-N-(3-(trifluoromethyl)benzyl)nonanamide,
9,9,9-trifluoro-8-oxo-N-(3-(trifluoromethoxy)phenyl)nonanamide,
N-(3,5-dimethoxybenzyl)-9,9,9-trifluoro-8-oxononanamide,
N-(2,4-dimethylphenyl)-9,9,9-trifluoro-8-oxononanamide,
N-(3,4-dimethylphenyl)-9,9,9-trifluoro-8-oxononanamide,
N-(3,5-dimethylphenyl)-9,9,9-trifluoro-8-oxononanamide,
N-(2,4-dimethoxyphenyl)-9,9,9-trifluoro-8-oxononanamide,
N-(2,5-dimethoxybenzyl)-9,9,9-trifluoro-8-oxononanamide,
N-(3,5-dimethoxyphenyl)-9,9,9-trifluoro-8-oxononanamide,
N-(1,3-benzodioxol-5-ylmethyl)-9,9,9-trifluoro-8-oxononanamide,
9,9,9-trifluoro-8-oxo-N-(3,4,5-trimethoxyphenyl)nonanamide,
N-(3,4-dichlorobenzyl)-9,9,9-trifluoro-8-oxononanamide,
8-((1,1′-biphenyl)-4-yloxy)-1,1,1-trifluoro-3-octyn-2-one,
N-(4-(dimethylamino)benzyl)-9,9,9-trifluoro-8-oxononanamide,
(3E)-8-((1,1′-biphenyl)-4-yloxy)-1,1,1-trifluoro-3-octen-2-one,
(8E)-9-(1,1′-biphenyl)-4-yl-1,1,1-trifluoro-8-nonen-2-one,
1-(2-(4-((1,1′-biphenyl)-4-yloxy)butyl)cyclopropyl)-2,2,2-trifluoroethanone,
9H-fluoren-9-ylmethyl 7,7,7-trifluoro-6-oxoheptylcarbamate,
4-((1,1′-biphenyl)-4-yloxy)-N-(3,3,3-trifluoro-2-oxopropyl)butanamide,
methyl 8-((1,1′-biphenyl)-4-yloxy)-2-oxooctanoate,
7-((1,1′-biphenyl)-3-yloxy)-1-(1,3-oxazol-2-yl)-1-heptanone,
8-((1,1′-biphenyl)-4-yloxy)-2-oxooctanoic acid,
ethyl 7-((1,1′-biphenyl)-4-yloxy)-2-oxoheptanoate,
ethyl 7-((3-bromo(1,1′-biphenyl)-4-yl)oxy)-2-oxoheptanoate,
8-(1,3-oxazol-2-yl)-8-oxo-N-phenyloctanamide,
N-(1,1′-biphenyl)-3-yl-8-(1,3-oxazol-2-yl)-8-oxooctanamide,
N-(4-chlorophenyl)-8-(1,3-oxazol-2-yl)-8-oxooctanamide,
8-(1,3-oxazol-2-yl)-8-oxo-N-(4-phenoxyphenyl)octanamide,
8-(1,3-oxazol-2-yl)-8-oxo-N-(2-pyridinyl)octanamide,
8-((1,1′-biphenyl)-4-yloxy)-N-methyl-2-oxooctanamide,
1-(1,3-oxazol-2-yl)-7-(phenylsulfanyl)-1-heptanone,
1-(1,3-oxazol-2-yl)-7-(phenylsulfonyl)-1-heptanone,
7-(2-naphthylsulfanyl)-1-(1,3-oxazol-2-yl)-1-heptanone,
7-(2-naphthylsulfonyl)-1-(1,3-oxazol-2-yl)-1-heptanone,
N-methyl-8-(2-naphthylsulfanyl)-2-oxooctanamide,
N-methyl-8-(2-naphthylsulfonyl)-2-oxooctanamide,
8-((1,1′-biphenyl)-4-ylsulfanyl)-N-methyl-2-oxooctanamide,
8-((1,1′-biphenyl)-4-ylsulfonyl)-N-methyl-2-oxooctanamide,
8-((1,1′-biphenyl)-4-ylsulfonyl)-N-methyl-2-oxooctanamide,
N-(6-(1 ,3-oxazol-2-yl)-6-oxohexyl)-1H-indole-2-carboxanmide,
7-((1,1′-biphenyl)-4-yloxy)-1-(4,5-dihydro-1,3-oxazol-2-yl)-1-heptanone,
9-((1,1′-biphenyl)-4-yloxy)-2,3-nonanedione,
N-(1,1′-biphenyl)-4-yl-7,8-dioxononanamide,
methyl 7-((1,1′-biphenyl)-4-yloxy)-2-oxoheptanoate,
methyl 9-((1,1′-biphenyl)-3-ylamino)-2,9-dioxononanoate,
methyl 9-anilino-2,9-dioxononanoate,
methyl 8-((4′-cyano(1,1′-biphenyl)-4-yl)oxy)-2-oxooctanoate,
8-((4′-cyano(1,1′-biphenyl)-4-yl)oxy)-N-methyl-2-oxooctanamide,
N⁹-(1,1′-biphenyl)-3-yl-N -methyl-2-oxononanediamide,
(7E)-8-(1,1′-biphenyl)-4-yl-1-(1 ,3-oxazol-2-yl)-7-octen-1-one,
4-((1E)-8-(1,3-oxazol-2-yl)-8-oxo-1-octenyl)benzonitrile,
N⁹-(1,1′-biphenyl)-3-yl-2-oxononanediamide,
N¹-methyl-2-oxo-N⁹-(4-phenyl-1 ,3-thiazol-2-yl)nonanediamide,
N¹-methyl-2-oxo-N⁹-(4-phenoxyphenyl)nonanediamide,
N⁹-(4,5-di phenyl-1,3-hiazol-2-yl)-N¹-methyl-2-oxononanediamide,
N⁹-(4-(3-methoxyphenyl)-1,3-thiazol-2-yl)-N -methyl-2-oxononanediamide,
N⁹-(4-(2-methoxyphenyl)-1,3-thiazol-2-yl)-N -methyl-2-oxononanediamide,
N¹-methyl-N⁹-(5-methyl-4-phenyl-1,3-thiazol-2-yl)-2-oxononanediamide,
N¹-methyl-2-oxo-N⁹-(4′-(trifluoromethoxy)(,1′-biphenyl)-3-yl)nonanediamide,
N⁹-(4-(4-chlorophenoxy)phenyl)-N¹-methyl-2-oxononanediamide,
N⁹-(4-methoxy(1,1′-biphenyl)-3-yl)-N¹-methyl-2-oxononanediamide,
N⁹-(4′-cyano(1,1′-biphenyl)-3-yl)-N¹-methyl-2-oxononanediamide,
N⁹-(6-methoxy(1,1′-biphenyl)3yl)-N¹-methyl-2-oxononanediamide,
N⁹-(1,1′biphenyl)-4-yl-N¹-methyl-2-oxononanediamide,
N⁹-(3,4dichlorophenyl)-N¹-methyl-2-oxononanediamide,
N⁹-methyl-2-oxo-N -(4-(trifluoromethyl)phenyl)nonanediamide,
N⁹-(3-cyanophenyl)-N¹-methyl-2-oxononanediamide, and
N⁹-(4-(4-methoxyphenyl)-1,3-thiazol-2-yl)-N¹-methyl-2-oxononanediamide.

38. A compound which is
methyl 8-((4′-cyano(1,1′-biphenyl)-4-yl)oxy)-3-hydroxy-2-oxooctanoate.
